SEQUENCE LISTING (6 sequences)

SEQ ID NO: 1 - DNA, 25 bp, Artificial
  Synthetic DNA
gcggtgttctgttggtgctgatatt

SEQ ID NO: 2 - DNA, 80 bp, Artificial
  Synthetic DNA
acgaattcgagctcggtacccggggatcctctagagtcgacctgcaggcatgcaagcttggcactggccgtcgttttaca

SEQ ID NO: 3 - DNA, 77 bp, Artificial
  Synthetic DNA
  misc_feature (52)..(52): N corresponds to a spacer comprising iSpC3.
agccagcgtccggggatgagctactcccggtttttttttttttttttttttngcggtgttctgttggtgctgatatt

SEQ ID NO: 4 - DNA, 147 bp, Artificial
  Synthetic DNA
ttttttttttttttttttttttttttttttttttttttttttttttttttttcggctccactctgaatgggttagggtcagggcacgggctagggactgggatagggttattctgtccgggagtagctcatccccggacgctggctt

SEQ ID NO: 5 - DNA, 48 bp, Artificial
  Synthetic DNA
acagaataaccctatcccagtccctagcccgtgccctgaccctaaccc

SEQ ID NO: 6 - DNA, 23 bp, Artificial
  Synthetic DNA
tatcagcaccaacagaacaccgc

(19) United States
(12) Patent Application Publication
(10) Pub. No.: US 2023/0220450 A1
(43) Pub. Date: Jul. 13, 2023

(54) ADAPTER MOLECULE, BIOMOLECULE-ADAPTER MOLECULE COMPLEX COMPOSED OF ADAPTER MOLECULE AND BIOMOLECULE BOUND TOGETHER, BIOMOLECULE ANALYZER AND BIOMOLECULE ANALYSIS METHOD

(71) Applicant: Hitachi High-Tech Corporation, Tokyo (JP)

(72) Inventors: Rena AKAHORI, Tokyo (JP); Yusuke GOTO, Tokyo (JP); Michiru FUJIOKA, Tokyo (JP); Itaru YANAGI, Tokyo (JP)

(73) Assignee: Hitachi High-Tech Corporation, Tokyo (JP)

(21) Appl. No.: 17/761,833

(22) PCT Filed: Sep. 18, 2019
(86) PCT No.: PCT/JP2019/036512

§ 371 Date: Mar. 18, 2022

(51) Int. Cl. C12Q 1/6825; C12Q 1/6869; G01N 27/327; G01N 27/447

(52) U.S. Cl. CPC C12Q 1/6825; C12Q 1/6869; G01N 27/3276; G01N 27/3278; G01N 27/44791

(57) ABSTRACT

A biomolecule is more easily and reliably reciprocated in a nanopore. An adapter molecule that directly or indirectly binds to a biomolecule to be analyzed comprises a three-dimensional structure formation domain consisting of a single-stranded nucleotide.

The Substitute Sequence Listing titled “Substitute Sequence Listing,” submitted Sep. 12, 2022, and having of file size of 1,809 bytes is incorporated herein by reference as if fully set forth.

### TECHNICAL FIELD

The present invention relates to an adapter molecule used for analysis of a biomolecule such as a nucleic acid, a biomolecule-adapter molecule complex in which the adapter molecule is bound, a biomolecule analyzer, and a biomolecule analysis method.

### BACKGROUND ART

Biomolecules such as proteins and nucleic acid molecules each have a structure in which monomers such as amino acids and nucleotides are linked to each other. In the proteins of these biomolecules, a monomer sequence is determined using a device that automates the Edman method (called a peptide sequencer or a protein sequencer). As a device for determining a monomer sequence (base sequence) of a nucleic acid molecule, the first-generation sequencer to which the Sanger method or the Maxam-Gilbert method is applied and the second-generation sequencer using a method combined with a pyrosequencing method or a bridge PCR method and a sequencing-by-synthesis (SBS) technology are known.

On the other hand, in the field of the next-generation DNA sequencer, a method of electrically and directly measuring a base sequence of DNA without carrying out an extension reaction or a fluorescent labeling has attracted attention. Specifically, research and development on a so-called nanopore DNA sequencing method in which DNA strands are directly measured without using reagents and a base sequence is determined have been actively promoted.

In the nanopore DNA sequencing method, a base sequence is measured by measuring a blocking current generated by DNA strands passing through a pore (hereinafter, referred to as a “nanopore”) formed in a thin film. That is, since the blocking current is changed by a difference in individual base types contained in the DNA strands, the base types can be sequentially identified by measuring the amount of the blocking current. In this method, unlike the various sequencers described above, it is not required to add an amplification reaction by an enzyme using a DNA strand as a template or a labeling substance such as a phosphor. Therefore, the nanopore DNA sequencing method has a high output and low running cost and enables long-base DNA deciphering in comparison to various sequencers according to the related art.

The nanopore DNA sequencing method is generally realized by a device for biomolecule analysis that comprises first and second liquid tanks filled with an electrolyte solution; a thin film partitioning the first and second liquid tanks and having a nanopore; and first and second electrodes provided in the first and second liquid tanks, respectively. The device for biomolecule analysis can also be implemented by an array device. The array device refers to a device comprising a plurality of sets of liquid chambers partitioned by a thin film. For example, the first liquid tank can be a common tank, and the second liquid tank can be a plurality of individual tanks. In this case, an electrode is arranged in each of the common tank and the individual tank.

In the configuration, a voltage is applied between the first liquid tank and the second liquid tank, and an ion current corresponding to a nanopore diameter flows through the nanopore. In addition, a potential gradient is generated in the nanopore in accordance with the applied voltage. When a biomolecule is introduced into the first liquid tank, the biomolecule is transferred to the second liquid tank through the nanopore according to a diffusion phenomenon and the generated potential gradient. A magnitude of the ion current is proportional to a cross-sectional area of the nanopore as a primary approximation. When DNA passes through the nanopore, the ion current is reduced because the nanopore is blocked by the DNA and an effective cross-sectional area is reduced. This current is referred to as a blocking current. Based on the magnitude of the blocking current, a difference between a single strand and double strand of DNA and the type of base are determined.

In addition, there is also known a method in which a pair of probe electrodes facing each other are provided on an inner side surface of a nanopore and the like and a voltage is applied between the electrodes so as to measure a tunnel current between DNA and the probe electrodes when the DNA passes through the nanopore and to determine the type of base based on a magnitude of the tunnel current.

As one of objects of the nanopore DNA sequencing method, conveyance control of DNA passing through the nanopore is exemplified. It is considered that, in order to measure the difference between types of individual bases contained in a DNA strand by the amount of the blocking current, a passing speed of DNA through the nanopore is required to be 100 μs or more per base, based on a current noise and a time constant of fluctuation of DNA molecules during measurement. However, the passing speed of DNA through the nanopore is to fast, usually 1 μs or less per base, that it is difficult to sufficiently measure the blocking current derived from each base.

As one of conveyance control methods, there is a method of utilizing a force to convayance control of single-stranded DNA as a template when DNA polymerase carries out a complementary strand synthesis reaction or when helicase dissociates double-stranded DNA (for example, see NPL 1). DNA polymerase binds to the DNA used as a template and performs a complementary strand synthesis reaction from an end of a primer complementarily bound to the template DNA. In the first liquid tank, the DNA polymerase carries out a complementary strand synthesis reaction in the vicinity of the nanopore, such that the template DNA is conveyed to the second liquid tank through the nanopore. The DNA polymerase or helicase is called a molecular motor.

In addition, as described in PTL 1, measurement accuracy can be improved by reciprocating single-stranded DNA to be analyzed between the first liquid tank and the second liquid tank through the nanopore. That is, the single-stranded DNA to be analyzed is reciprocated between the first liquid tank and the second liquid tank, and measurement is performed a plurality of times, such that an error caused in a single measurement can be corrected. In this case, as described in PTL 1, by binding a first stopper molecule (larger than a nanopore diameter) to one end of the single-stranded DNA to be analyzed, the single-stranded DNA is transferred from the other end of the single-stranded DNA to the second liquid tank through the nanopore, and a second stopper molecule (larger than the nanopore diameter) is bound to the other end of the single-stranded DNA in the second liquid tank. Therefore, one end of the single-stranded DNA can stay in the first liquid tank, the other end of the single-stranded DNA can stay in the second liquid tank, and it is possible to prevent the single-stranded DNA from falling out from the nanopore during the reciprocating motion.

### CITATION LIST

### Non-Patent Literature

NPL 1: Gerald M Cherf et al., Nat. Biotechnol. 30, No. 4, p. 349-353, 2012

### Patent Literature

PTL 1: JP 5372570 B2

### SUMMARY OF INVENTION

### Technical Problem

As described above, the biomolecule is reciprocated between the first liquid tank and the second liquid tank through the nanopore to improve reading accuracy. However, the reciprocating motion through the nanopore, that is, the conveyance control of the biomolecule, is technically very difficult. Therefore, a technology for more easily and reliably reciprocating a biomolecule has been required.

In view of the circumstances described above, an object of the present invention is to provide an adapter molecule capable of more simply and reliably reciprocating a biomolecule to be analyzed through a nanopore, a biomolecule-adapter molecule complex composed of the adapter molecule and a biomolecule bound together, a biomolecule analyzer, and a biomolecule analysis method.

### Solution to Problem

The present invention that has achieved the object described above includes the following.

(1) An adapter molecule that directly or indirectly binds to a biomolecule to be analyzed, comprising a three-dimensional structure formation domain consisting of a single-stranded nucleotide.

(2) The adapter molecule according to (1), further comprising: a double-stranded nucleic acid region consisting of base sequences complementary to each other and comprising one end that directly or indirectly binds to the biomolecule to be analyzed; and a single-stranded nucleic acid region linked to an other end different from the one end of the double-stranded nucleic acid region and comprising the three-dimensional structure formation domain.

(3) The adapter molecule according to (1), further comprising: a double-stranded nucleic acid region consisting of base sequences complementary to each other and comprising one end that directly or indirectly binds to the biomolecule to be analyzed; and a pair of single-stranded nucleic acid regions linked to an other end different from the one end of the double-stranded nucleic acid region and consisting of base sequences not complementary to each other, in which the three-dimensional structure formation domain is located in a single-stranded nucleic acid region having a 5′-terminus of the pair of single-stranded nucleic acid regions.

(4) The adapter molecule according to (1), further comprising a three-dimensional structure formation inhibiting oligomer comprising a base sequence complementary to at least a portion of the three-dimensional structure formation domain.

(5) The adapter molecule according to (4), in which the three-dimensional structure formation inhibiting oligomer hybridizes with at least a portion of the three-dimensional structure formation domain, and a side of a terminus from a portion with which the three-dimensional structure formation inhibiting oligomer hybridizes is a single strand.

(6) The adapter molecule according to (3), in which a single-stranded nucleic acid region having a 3′-terminus at an end of the pair of single-stranded nucleic acid regions comprises a fall-off prevention portion having a diameter larger than that of a nanopore in an analyzer for the biomolecule.

(7) The adapter molecule according to (6), in which the fall-off prevention portion is a molecule bondable to the single-stranded nucleic acid region or a hairpin structure formed in a complementary region in the single-stranded nucleic acid region.

(8) The adapter molecule according to (3), in which a single-stranded nucleic acid region having a 3′-terminus at an end of the pair of single-stranded nucleic acid regions comprises a molecular motor binding portion to which a molecular motor binds.

(9) The adapter molecule according to (8), in which the single-stranded nucleic acid region comprising the molecular motor binding portion comprises a primer binding portion, with which a primer hybridizes, on a side of the 3′-terminus from the molecular motor binding portion.

(10) The adapter molecule according to (9), further comprising a spacer to which the molecular motor is not bound being provided between the molecular motor binding portion and the primer binding portion.

(11) An adapter molecule that directly or indirectly binds to a biomolecule to be analyzed and consists of a single-stranded nucleotide, the adapter molecule comprising a plurality of sets of a molecular motor binding portion to which a molecular motor binds and a primer binding portion with which a primer hybridizes on a side of the 3′-terminus from the molecular motor binding portion.

(12) The adapter molecule according to (11) , further comprising a spacer to which the molecular motor is not bound being provided between the molecular motor binding portion and the primer binding portion.

(13) The adapter molecule according to (11) , further comprising a fall-off prevention portion having a diameter larger than that of a nanopore in an analyzer for the biomolecule at an end opposite to an end that directly or indirectly binds to the biomolecule.

(14) The adapter molecule according to (13), in which the fall-off prevention portion is a molecule bondable to the single-stranded nucleic acid region or a hairpin structure formed in a complementary region in the single-stranded nucleic acid region.

(15) The adapter molecule according to (11) , further comprising: a double-stranded nucleic acid region consisting of base sequences complementary to each other and comprising one end that directly or indirectly binds to the biomolecule to be analyzed; and a single-stranded nucleic acid region linked to an other end different from the one end of the double-stranded nucleic acid region, having the 3′-terminus, and comprising a plurality of sets of the molecular motor binding portion and the primer binding portion.

(16) The adapter molecule according to (11) , further comprising: a double-stranded nucleic acid region consisting of base sequences complementary to each other and comprising one end that directly or indirectly binds to the biomolecule to be analyzed; and a pair of single-stranded nucleic acid regions linked to an other end different from the one end of the double-stranded nucleic acid region and consisting of base sequences not complementary to each other, in which the plurality of sets of the molecular motor binding portion and the primer binding portion are located in a single-stranded nucleic acid region having the 3′-terminus of the pair of single-stranded nucleic acid regions.

(17) The adapter molecule according to (16), in which a single-stranded nucleic acid region having a 5′-terminus of the pair of single-stranded nucleic acid regions comprises a three-dimensional structure formation domain.

(18) The adapter molecule according to (17), further comprising a three-dimensional structure formation inhibiting oligomer comprising a base sequence complementary to at least a portion of the three-dimensional structure formation domain.

(19) The adapter molecule according to (18), in which the three-dimensional structure formation inhibiting oligomer hybridizes with at least a portion of the three-dimensional structure formation domain, and a side of a terminus from a portion with which the three-dimensional structure formation inhibiting oligomer hybridizes is a single strand.

(20) The adapter molecule according to (16), in which a single-stranded nucleic acid region having a 5′-terminus of the pair of single-stranded nucleic acid regions comprises a molecular motor detachment induction portion of which a binding force to a molecular motor is smaller than that of the biomolecule.

(21) An adapter molecule that directly or indirectly binds to a biomolecule to be analyzed, comprising a molecular motor detachment induction portion of which a binding force to a molecular motor is smaller than that of the biomolecule.

(22) The adapter molecule according to (21), in which the molecular motor detachment induction portion is a carbon chain that does not contain a phosphodiester bond or an abasic sequence portion.

(23) The adapter molecule according to (21) , further comprising a three-dimensional structure formation domain consisting of a single-stranded nucleotide on a side of a 5′-terminus from the molecular motor detachment induction portion.

(24) The adapter molecule according to (21) , further comprising: a double-stranded nucleic acid region consisting of base sequences complementary to each other and comprising one end that directly or indirectly binds to the biomolecule to be analyzed; and

a single-stranded nucleic acid region linked to an other end different from the one end of the double-stranded nucleic acid region, having a 5′-terminus, and comprising the molecular motor detachment induction portion.

(25) The adapter molecule according to (21) , further comprising: a double-stranded nucleic acid region consisting of base sequences complementary to each other and comprising one end that directly or indirectly binds to the biomolecule to be analyzed; and a pair of single-stranded nucleic acid regions linked to an other end different from the one end of the double-stranded nucleic acid region and consisting of base sequences not complementary to each other, in which the molecular motor detachment induction portion is located in a single-stranded nucleic acid region having a 5′-terminus of the pair of single-stranded nucleic acid regions.

(26) The adapter molecule according to (23) , further comprising a three-dimensional structure formation inhibiting oligomer comprising a base sequence complementary to at least a portion of the three-dimensional structure formation domain.

(27) The adapter molecule according to (26), in which the three-dimensional structure formation inhibiting oligomer hybridizes with at least a portion of the three-dimensional structure formation domain, and a side of a terminus from a portion with which the three-dimensional structure formation inhibiting oligomer hybridizes is a single strand.

(28) The adapter molecule according to (25), in which a single-stranded nucleic acid region having a 3′-terminus at an end of the pair of single-stranded nucleic acid regions includes a fall-off prevention portion having a diameter larger than that of a nanopore in an analyzer for the biomolecule.

(29) The adapter molecule according to (28), in which the fall-off prevention portion is a molecule bondable to the single-stranded nucleic acid region or a hairpin structure formed in a complementary region in the single-stranded nucleic acid region.

(30) The adapter molecule according to (25), in which a single-stranded nucleic acid region having a 3′-terminus at an end of the pair of single-stranded nucleic acid regions comprises a molecular motor binding portion to which a molecular motor binds.

(31) The adapter molecule according to (30), in which the single-stranded nucleic acid region comprising the molecular motor binding portion comprises a primer binding portion with which a primer hybridizes on a side of the 3′-terminus from the molecular motor binding portion.

(32) The adapter molecule according to (31) , further comprising a spacer to which the molecular motor is not bound being provided between the molecular motor binding portion and the primer binding portion.

(33) The adapter molecule according to (25), in which a single-stranded nucleic acid region having a 3′-terminus at an end of the pair of single-stranded nucleic acid regions comprises a plurality of sets of a molecular motor binding portion to which a molecular motor binds and a primer binding portion with which a primer hybridizes on a side of the 3′-terminus from the molecular motor binding portion.

(34) The adapter molecule according to (33), further comprising a spacer to which the molecular motor is not bound being provided between the molecular motor binding portion and the primer binding portion.

(35) A biomolecule-adapter molecule complex, comprising a biomolecule to be analyzed and the adapter molecule directly or indirectly bound to at least one terminus of the biomolecule according to any one of (1) to (10).

(36) A biomolecule-adapter molecule complex, comprising a biomolecule to be analyzed and the adapter molecule directly or indirectly bound to at least one terminus of the biomolecule according to any one of (11) to (20).

(37) A biomolecule-adapter molecule complex, comprising a biomolecule to be analyzed and the adapter molecule directly or indirectly bound to at least one terminus of the biomolecule according to any one of (21) to (34).

(38) A biological analyzer comprising: a thin film having a nanopore; a first liquid tank and a second liquid tank facing each other with the thin film interposed therebetween; a voltage source for applying a voltage between the first liquid tank and the second liquid tank, in a state where the first liquid tank is filled with an electrolyte solution comprising the biomolecule-adapter molecule complex according to (35), (36), or (37) and the second liquid tank is filled with an electrolyte solution; and a controller for controlling the voltage source to generate a desired potential gradient between the first liquid tank and the second liquid tank.

(39) A biomolecule analysis method comprising steps of: applying a voltage between a first liquid tank and a second liquid tank facing each other with a thin film having a nanopore interposed therebetween in a state where the first liquid tank is filled with an electrolyte solution comprising the biomolecule-adapter molecule complex according to (35) and the second liquid tank is filled with an electrolyte solution to generate a potential gradient at which the first liquid tank is set to a negative or ground potential and the second liquid tank is set to a positive potential; forming a three-dimensional structure in the second liquid tank by the three-dimensional structure formation domain of the adapter molecule; and measuring a signal generated when the biomolecule-adapter molecule complex is convayed between the second liquid tank and the first liquid tank through the nanopore, in which in the step of forming of the potential gradient, the three-dimensional structure formation domain in the biomolecule-adapter molecule complex is introduced into the second liquid tank through the nanopore, and the biomolecule-adapter molecule complex is convayed from the first liquid tank to the second liquid tank due to the potential gradient.

(40) A biomolecule analysis method comprising steps of: applying a voltage between a first liquid tank and a second liquid tank facing each other with a thin film having a nanopore interposed therebetween in a state where the first liquid tank is filled with an electrolyte solution comprising the biomolecule-adapter molecule complex according to (36), a molecular motor boundable to a molecular motor binding portion of the adapter molecule, and a primer capable of hybridizing with a primer binding portion of the adapter molecule and the second liquid tank is filled with an electrolyte solution to generate a potential gradient at which the first liquid tank is set to a negative or ground potential and the second liquid tank is set to a positive potential; and measuring a signal generated when the biomolecule-adapter molecule complex is convayed between the second liquid tank and the first liquid tank through the nanopore, in which in the step of measuring of the signal, the measurement is repeated, where the measurement being performed by synthesizing a complementary strand from the primer hybridizing with the primer binding portion by the molecular motor located closest to the nanopore to convay the biomolecule-adapter molecule complex from the second liquid tank to the first liquid tank so as to measure a signal generated when the biomolecule-adapter molecule complex passes through the nanopore, and then, convaying the biomolecule-adapter molecule complex having a complementary strand from the first liquid tank to the second liquid tank to peel off the complementary strand and synthesizing the complementary strand by the molecular motor located closest to the nanopore again to covay the biomolecule-adapter molecule complex from the second liquid tank to the first liquid tank so as to measure a signal.

(41) A biomolecule analysis method comprising step of: applying a voltage between a first liquid tank and a second liquid tank facing each other with a thin film having a nanopore interposed therebetween in a state where the first liquid tank is filled with an electrolyte solution comprising the biomolecule-adapter molecule complex according to (37), a molecular motor boundable to a molecular motor binding portion of the biomolecule-adapter molecule complex, and a primer capable of hybridizing with a primer binding portion of the biomolecule-adapter molecule complex and the second liquid tank is filled with an electrolyte solution to generate a potential gradient at which the first liquid tank is set to a negative or ground potential and the second liquid tank is set to a positive potential; and measuring a signal generated when the biomolecule-adapter molecule complex is convayed between the second liquid tank and the first liquid tank through the nanopore, in which in the step of measuring of the signal, the molecular motor synthesizes a complementary strand from the primer hybridizing with the primer binding portion to convay the biomolecule-adapter molecule complex from the second liquid tank to the first liquid tank and separates at the molecular motor detachment induction portion in the biomolecule-adapter molecule complex.

### Advantageous Effects of Invention

According to the adapter molecule, the biomolecule-adapter molecule complex composed of the adapter molecule and a biomolecule bound together, the biomolecule analyzer, and the biomolecule analysis method according to the present invention, the biomolecule and the adapter molecule can reliably reciprocate in the nanopore by using a characteristic adapter molecule. Therefore, the biomolecule can be accurately analyzed.

### BRIEF DESCRIPTION OF DRAWINGS

FIG. 1 is a configuration view schematically illustrating a biomolecule analyzer using an adapter molecule to which the present invention is applied.

FIG. 2 is a configuration view schematically illustrating a configuration of a biomolecule-adapter molecule complex comprising an adapter molecule to which the present invention is applied.

FIG. 3 is a configuration view schematically illustrating a step of analyzing the biomolecule-adapter molecule complex comprising the adapter molecule illustrated in FIG. 2.

FIG. 4 is a configuration view schematically illustrating a step of analyzing the biomolecule-adapter molecule complex comprising the adapter molecule to which the present invention is applied, followed by the step illustrated in FIG. 3.

FIG. 5 is a configuration view schematically illustrating a step of analyzing the biomolecule-adapter molecule complex comprising the adapter molecule to which the present invention is applied, followed by the step illustrated in FIG. 4.

FIG. 6 is a configuration view schematically illustrating a step of analyzing the biomolecule-adapter molecule complex comprising the adapter molecule to which the present invention is applied using a molecular motor.

FIG. 7 is a configuration view schematically illustrating a step of analyzing the biomolecule-adapter molecule complex comprising the adapter molecule to which the present invention is applied using a molecular motor, followed by the step illustrated in FIG. 6.

FIG. 8 is a configuration view schematically illustrating a step of analyzing the biomolecule-adapter molecule complex comprising the adapter molecule to which the present invention is applied using a molecular motor, followed by the step illustrated in FIG. 7.

FIG. 9 is a configuration view schematically illustrating a configuration of a biomolecule-adapter molecule complex comprising another adapter molecule to which the present invention is applied.

FIG. 10 is a configuration view schematically illustrating a step of analyzing the biomolecule-adapter molecule complex comprising the adapter molecule illustrated in FIG. 9.

FIG. 11 is a configuration view schematically illustrating a step of analyzing the biomolecule-adapter molecule complex comprising another adapter molecule to which the present invention is applied, followed by the step illustrated in FIG. 10.

FIG. 12A is a configuration view schematically illustrating a step of analyzing the biomolecule-adapter molecule complex comprising another adapter molecule to which the present invention is applied, followed by the step illustrated in FIG. 11.

FIG. 12B is a configuration view schematically illustrating a state where the biomolecule-adapter molecule complex is convayed in an opposite direction from the state illustrated in FIG. 12A.

FIG. 13 is a configuration view illustrating a configuration of still another adapter molecule to which the present invention is applied.

FIG. 14A is a configuration view schematically illustrating a step of analyzing the biomolecule-adapter molecule complex comprising the adapter molecule illustrated in FIG. 13.

FIG. 14B is a configuration view schematically illustrating a step of analyzing the biomolecule-adapter molecule complex comprising the adapter molecule illustrated in FIG. 13, followed by the step illustrated in FIG. 14A.

FIG. 15A is a configuration view schematically illustrating a step of analyzing the biomolecule-adapter molecule complex comprising the adapter molecule illustrated in FIG. 13, followed by the step illustrated in FIG. 14B.

FIG. 15B is a configuration view schematically illustrating a step of analyzing the biomolecule-adapter molecule complex comprising the adapter molecule illustrated in FIG. 13, followed by the step illustrated in FIG. 15A.

FIG. 15C is a configuration view schematically illustrating a step of analyzing the biomolecule-adapter molecule complex comprising the adapter molecule illustrated in FIG. 13, followed by the step illustrated in FIG. 15B.

FIG. 15D is a configuration view schematically illustrating a step of analyzing the biomolecule-adapter molecule complex comprising the adapter molecule illustrated in FIG. 13, followed by the step illustrated in FIG. 15C.

FIG. 15E is a configuration view schematically illustrating a step of analyzing the biomolecule-adapter molecule complex comprising the adapter molecule illustrated in FIG. 13, followed by the step illustrated in FIG. 15D.

FIG. 15F is a configuration view schematically illustrating a step of analyzing the biomolecule-adapter molecule complex comprising the adapter molecule illustrated in FIG. 13, followed by the step illustrated in FIG. 15E.

FIG. 15G is a configuration view schematically illustrating a step of analyzing the biomolecule-adapter molecule complex comprising the adapter molecule illustrated in FIG. 13, followed by the step illustrated in FIG. 15F.

FIG. 16 is a configuration view schematically illustrating a biomolecule analyzer using another adapter molecule to which the present invention is applied.

FIG. 17 is a configuration view schematically illustrating a configuration of a biomolecule-adapter molecule complex comprising another adapter molecule to which the present invention is applied.

FIG. 18 is a configuration view schematically illustrating a step of analyzing the biomolecule-adapter molecule complex comprising the adapter molecule illustrated in FIG. 17.

FIG. 19 is a configuration view schematically illustrating a step of analyzing the biomolecule-adapter molecule complex comprising the adapter molecule illustrated in FIG. 17, followed by the step illustrated in FIG. 18.

FIG. 20 is a configuration view schematically illustrating a step of analyzing the biomolecule-adapter molecule complex comprising the adapter molecule illustrated in FIG. 17, followed by the step illustrated in FIG. 19.

FIG. 21 is a configuration view schematically illustrating a step of analyzing the biomolecule-adapter molecule complex comprising the adapter molecule illustrated in FIG. 17, followed by the step illustrated in FIG. 20.

FIG. 22 is a configuration view illustrating a configuration of still another adapter molecule to which the present invention is applied.

FIG. 23 is a configuration view schematically illustrating a step of analyzing the biomolecule-adapter molecule complex comprising the adapter molecule illustrated in FIG. 22.

FIG. 24 is a configuration view schematically illustrating a step of analyzing the biomolecule-adapter molecule complex comprising the adapter molecule illustrated in FIG. 22, followed by the step illustrated in FIG. 23.

FIG. 25 is a configuration view schematically illustrating a step of analyzing the biomolecule-adapter molecule complex comprising the adapter molecule illustrated in FIG. 22, followed by the step illustrated in FIG. 24.

FIG. 26 is a configuration view schematically illustrating a step of analyzing the biomolecule-adapter molecule complex comprising the adapter molecule illustrated in FIG. 22, followed by the step illustrated in FIG. 25.

FIG. 27 is a configuration view schematically illustrating a step of analyzing the biomolecule-adapter molecule complex comprising the adapter molecule illustrated in FIG. 22, followed by the step illustrated in FIG. 26.

FIG. 28 is a configuration view schematically illustrating a step of analyzing the biomolecule-adapter molecule complex comprising still another adapter molecule to which the present invention is applied.

FIG. 29 is a configuration view schematically illustrating a step of analyzing the biomolecule-adapter molecule complex, followed by the step illustrated in FIG. 28.

FIG. 30 is a configuration view schematically illustrating a step of analyzing the biomolecule-adapter molecule complex, followed by the step illustrated in FIG. 29.

FIG. 31 is a configuration view schematically illustrating a step of analyzing the biomolecule-adapter molecule complex, followed by the step illustrated in FIG. 30.

FIG. 32 is a configuration view schematically illustrating a step of analyzing the biomolecule-adapter molecule complex, followed by the step illustrated in FIG. 31.

FIG. 33 is a configuration view schematically illustrating a step of analyzing the biomolecule-adapter molecule complex, followed by the step illustrated in FIG. 32.

FIG. 34 is a configuration view schematically illustrating a step of analyzing the biomolecule-adapter molecule complex, followed by the step illustrated in FIG. 33.

FIG. 35 is a configuration view schematically illustrating a biomolecule analyzer using still another adapter molecule to which the present invention is applied.

FIG. 36 is a configuration view schematically illustrating a configuration of a biomolecule-adapter molecule complex comprising still another adapter molecule to which the present invention is applied.

FIG. 37 is a configuration view schematically illustrating a step of analyzing the biomolecule-adapter molecule complex illustrated in FIG. 36.

FIG. 38 is a configuration view schematically illustrating a step of analyzing the biomolecule-adapter molecule complex illustrated in FIG. 36, followed by the step illustrated in FIG. 37.

FIG. 39 is a configuration view schematically illustrating a step of analyzing the biomolecule-adapter molecule complex illustrated in FIG. 36, followed by the step illustrated in FIG. 38.

FIG. 40 is a configuration view illustrating a configuration of still another adapter molecule to which the present invention is applied.

FIG. 41 is a configuration view schematically illustrating a step of analyzing the biomolecule-adapter molecule complex comprising the adapter molecule illustrated in FIG. 40.

FIG. 42 is a configuration view schematically illustrating a step of analyzing the biomolecule-adapter molecule complex comprising the adapter molecule illustrated in FIG. 40, followed by the step illustrated in FIG. 41.

FIG. 43 is a configuration view schematically illustrating a step of analyzing the biomolecule-adapter molecule complex comprising the adapter molecule illustrated in FIG. 40, followed by the step illustrated in FIG. 42.

FIG. 44 is a configuration view schematically illustrating a step of analyzing the biomolecule-adapter molecule complex comprising the adapter molecule illustrated in FIG. 40, followed by the step illustrated in FIG. 43.

FIG. 45 is a configuration view schematically illustrating a step of analyzing the biomolecule-adapter molecule complex comprising the adapter molecule illustrated in FIG. 40, followed by the step illustrated in FIG. 44.

FIG. 46 is a configuration view illustrating a configuration of still another adapter molecule to which the present invention is applied.

FIG. 47 is a configuration view schematically illustrating a step of analyzing the biomolecule-adapter molecule complex comprising the adapter molecule illustrated in FIG. 46.

FIG. 48 is a configuration view schematically illustrating a step of analyzing the biomolecule-adapter molecule complex comprising the adapter molecule illustrated in FIG. 46, followed by the step illustrated in FIG. 47.

FIG. 49 is a configuration view schematically illustrating a step of analyzing the biomolecule-adapter molecule complex comprising the adapter molecule illustrated in FIG. 46, followed by the step illustrated in FIG. 48.

FIG. 50 is a configuration view illustrating a configuration of still another adapter molecule to which the present invention is applied.

FIG. 51 is a characteristic diagram illustrating relationships between an elapsed time and a blocking current measured in Reference Example 1.

FIG. 52 is a characteristic diagram illustrating changes in ion currents when an adapter having no telomere structure and an adapter having a telomere structure are measured.

FIG. 53 is a characteristic diagram illustrating results obtained by melting a single strand having a telomere structure in a measurement solution and measuring a blocking current of a nanopore.

FIG. 54 is a characteristic diagram illustrating results obtained by ligating an adapter molecule having a telomere structure to a biomolecule and measuring a blocking current using a sample containing streptavidin at the other terminus.

FIG. 55 is a characteristic diagram illustrating results obtained by ligating an adapter molecule having a telomere structure to a biomolecule and measuring a blocking current using another sample containing streptavidin at the other terminus.

FIG. 56a is a characteristic diagram illustrating results obtained by observing a nanopore passing signal using a template (without SA) comprising a molecular motor detachment induction portion in the presence of a molecular motor.

FIG. 56b is a characteristic diagram illustrating results obtained by observing a nanopore passing signal using a template (with SA) comprising a molecular motor detachment induction portion in the presence of a molecular motor.

FIG. 57 is a photograph showing results obtained by performing electrophoresis on an adapter molecule in which an interval between adjacent primer binding sites is changed in the presence or absence of a molecular motor.

FIG. 58 is a characteristic diagram illustrating results obtained by observing a nanopore passing signal in the presence of a molecular motor using an adapter molecule comprising a plurality of sets of a primer binding portion and a molecular motor binding portion, in which (a) is a representative diagram of measured blocking signals and (b) is a characteristic diagram illustrating results of Dotplot analysis.

### DESCRIPTION OF EMBODIMENTS

Hereinafter, an adapter molecule, a biomolecule-adapter molecule complex, a biomolecule analyzer, and a biomolecule analysis method according to the present invention will be described in detail with reference to the drawings. However, these drawings illustrate specific embodiments in accordance with the principles of the invention, and are provided for the understanding of the present invention, but are not used to limit the interpretation of the present invention.

Hereinafter, as a biomolecule analyzer described in all the embodiments, a biomolecule analyzer known in the art, which is used for analyzing a biomolecule by a so-called blocking current method, can be applied. Examples of a biomolecule analyzer known in the related art can include devices disclosed in U.S. Pat. No. 5,795,782, “Scientific Reports 4, 5000, 2014, Akahori, et al.”, “Nanotechnology 25(27):275501, 2014, Yanagi et al.”, “Scientific Reports, 5, 14656, 2015, Goto et al.”, and “Scientific Reports 5, 16640, 2015”.

### Embodiment 1-1

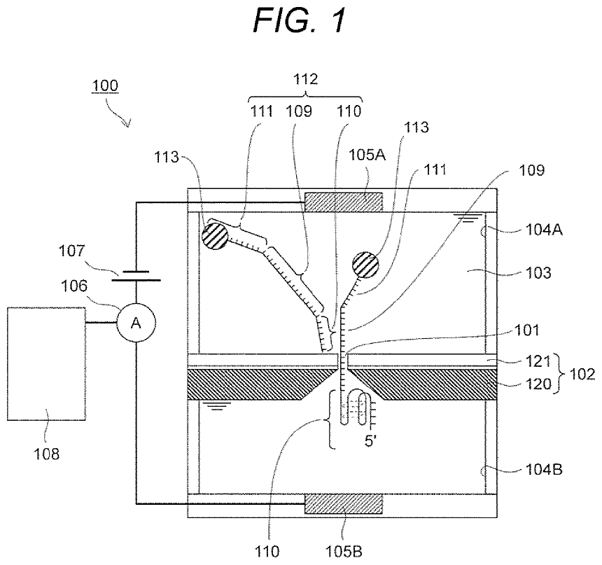

FIG. 1 illustrates a configuration example of a biomolecule analyzer 100 that analyzes a biomolecule-adapter molecule complex in which an adapter molecule and a biomolecule to be analyzed are directly or indirectly linked to each other. The biomolecule analyzer 100 illustrated in FIG. 1 is a device for biomolecule analysis that measures an ion current in a blocking current method, and comprises a substrate 102 in which a nanopore 101 is formed, a pair of liquid tanks 104 (a first liquid tank 104A and a second liquid tank 104B) arranged to be in contact with the substrate 102 with the substrate 102 interposed therebetween and filled with an electrolyte solution 103 therein, and a pair of electrodes 105 (a first electrode 105A and a second electrode 105B) in contact with the first liquid tank 104A and the second liquid tank 104B, respectively. At the time of measurement, a predetermined voltage is applied from a voltage source 107 between the pair of electrodes 105, and a current flows between the pair of electrodes 105. A magnitude of the current flowing between the electrodes 105 is measured by an ammeter 106, and a measured value thereof is analyzed by a computer 108.

For the electrolyte solution 103, for example, KCl, NaCl, LiCl, or CsCl is used. The electrolyte solution 103 may have the same compositions or different compositions in the first liquid tank 104A and the second liquid tank 104B. The first liquid tank 104A is filled with an electrolyte solution 103 containing a biomolecule-adapter molecule complex and the like described in detail below. In addition, a buffer can also be mixed in the electrolyte solution 103 in each of the first liquid tank 104A and the second liquid tank 104B in order to stabilize the biomolecule. As the buffer, Tris, EDTA, PBS, or the like is used. The first electrode 105A and the second electrode 105B can be formed of, for example, a conductive material such as Ag, AgCl, or Pt.

The electrolyte solution 103 filled in the first liquid tank 104A contains a biomolecule-adapter molecule complex 112 obtained by binding a first adapter molecule 110 and a second adapter molecule 111 to a biomolecule 109 to be analyzed. The first adapter molecule 110 and the second adapter molecule 111 can be bound to an end of the biomolecule 109 to be analyzed, and are nucleic acid molecules composed of nucleotides, pseudo nucleotides, peptide nucleic acids, and the like. The first adapter molecule 110 is bound to one end of the biomolecule 109 to be analyzed to form a three-dimensional structure in the second liquid tank 104B. The second adapter molecule 111 includes a fall-off prevention portion 113 at an end opposite to an end bound to the biomolecule 109.

Here, a three-dimensional structure formed by the first adapter molecule 110 in the second liquid tank 104B is not particularly limited, and refers to a three-dimensional structure having an outer shape larger than a diameter of the nanopore 101. Specific examples of the three-dimensional structure can include, but are not particularly limited to, a hairpin structure, a guanine quadruplex (G-quadruplex or G4, G-quartet) structure (for example, a telomere structure), a DNA nanoball structure, and a DNA origami structure. In addition, the three-dimensional structure may be a structure formed by hybridization in one molecule or formation of a chelate structure. Furthermore, as will be described in detail below, since a measurement voltage is applied to the three-dimensional structure in the vicinity of the nanopore 101, it is preferable that a withstand voltage for maintaining the three-dimensional structure is equal to or higher than the measurement voltage. However, even when the withstand voltage for maintaining the three-dimensional structure is lower than the measurement voltage, it is also possible to increase the withstand voltage by binding a protein or the like.

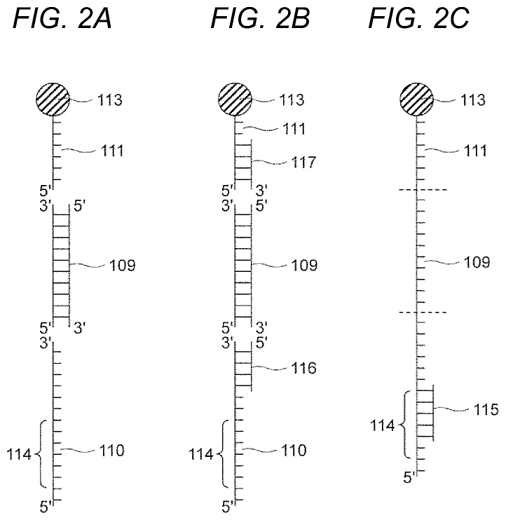

The biomolecule-adapter molecule complex 112 composed of single-stranded DNA as illustrated in FIG. 1 can be prepared by denaturing double-stranded DNA to be analyzed into single-stranded DNA, and then binding the first adapter molecule 110 and the second adapter molecule 111 of each single-stranded DNA to each other. Alternatively, as illustrated in FIG. 2(A), the first adapter molecule 110 is bound to one end of double-stranded DNA to be analyzed and the second adapter molecule 111 is bound to the other end, and then, the double-stranded DNA is denaturated, such that the biomolecule-adapter molecule complex 112 composed of single-stranded DNA may be prepared (FIG. 2(C)). In this case, the first adapter molecule 110 includes a three-dimensional structure formation domain 114 forming the three-dimensional structure described above in the molecule. That is, the three-dimensional structure formation domain 114 is a domain comprising a base sequence necessary for forming a three-dimensional structure such as a hairpin structure, a guanine quadruplex structure, a DNA nanoball structure, or a DNA origami structure as described above.

In addition, as illustrated in FIG. 2(C), the three-dimensional structure formation domain 114 preferably has a three-dimensional structure formation inhibiting oligomer 115 for preventing formation of a three-dimensional structure before being introduced into the second liquid tank 104B to form a three-dimensional structure. The three-dimensional structure formation inhibiting oligomer 115 can hybridize with at least a portion of the three-dimensional structure formation domain 114 to prevent formation of a three-dimensional structure by the three-dimensional structure formation domain 114. The three-dimensional structure formation inhibiting oligomer 115 may be a nucleotide chain that can hybridize with the entire three-dimensional structure formation domain 114, or may be a nucleotide chain that can hybridize with a portion of the three-dimensional structure formation domain 114, which is sufficient to prevent formation of a three-dimensional structure. For example, in a case where the three-dimensional structure formation domain 114 is a G-quadruplex structure, a nucleotide chain that can hybridize with guanine residues constituting the quadruplex can be used as the three-dimensional structure formation inhibiting oligomer 115. A base length of the three-dimensional structure formation inhibiting oligomer 115 can be 10 to about table number, and is more preferably a base length of 15 to 60.

In addition, as illustrated in FIG. 2(B), the first adapter molecule 110 and the second adapter molecule 111 may have a configuration in which double-stranded regions 116 and 117 are provided at least at ends boound to double-stranded DNA to be analyzed, respectively. Although not illustrated, all the first adapter molecule 110 and the second adapter molecule 111 may be double-strands. In any of these cases, the biomolecule-adapter molecule complex 112 composed of single-stranded DNA can be prepared by binding the first adapter molecule 110 and the second adapter molecule 111 to the double-stranded DNA to be analyzed, and then, denaturing the single strand (FIG. 2(C)).

Although not illustrated in FIG. 2(B), in the double-stranded regions 116 and 117 in the first adapter molecule 110 and the second adapter molecule 111, the end bound to the biomolecule 109 is preferably the 3′-protruding terminus (for example, the dT protruding terminus). By making the end the 3′-dT protruding terminus, it is possible to prevent formation of heterodimers or homodimers of the first adapter molecule 110 and the second adapter molecule 111 when the adapter molecule 110 and the biomolecule 109 are bound to each other.

Furthermore, in the first adapter molecule 110 and the second adapter molecule 111, the length and base sequence of the double-stranded regions 116 and 117 are not particularly limited, and can be any length and any base sequence. For example, the length of each of the double-stranded regions 116 and 117 can be a base length of 5 to 100, 10 to 80, 15 to 60, or 20 to 40.

In addition, although not illustrated, the first adapter molecule 110 and the second adapter molecule 111 may be indirectly bound to the biomolecule 109. The indirect linking means that the first adapter molecule 110 and the second adapter molecule 111 are bound to the biomolecule 109 via a nucleic acid fragment having a predetermined base length, and the first adapter molecule 110 and the second adapter molecule 111 are bound to the biomolecule 109 via a functional group introduced according to the type of the biomolecule 109.

In a case where the biomolecule 109 to be analyzed is double-stranded DNA fragment, one strand of the double-stranded DNA fragment is used as a reference, the first adapter molecule 110 binds to the 5′-terminus of the reference strand, and the second adapter molecule 111 binds to the 3′-terminus of the corresponding strand. However, this may be reversed, and the first adapter molecule 110 may bind to the 3′-terminus of the corresponding strand and the second adapter molecule 111 may bind to the 5′-terminus of the corresponding strand.

Here, the fall-off prevention portion 113 in the second adapter molecule 111 refers to a configuration having a function of preventing a single-stranded biomolecule-adapter molecule complex 112 existing in the first liquid tank 104A from falling out into the second liquid tank 104B through the nanopore 101. Therefore, as the molecule that can be used as the fall-off prevention portion 113, for example, a complex of an anti-DIG antibody against avidin, streptavidin, or Digoxigein (DIG) and beads or the like can be used.

In addition, it is preferable that the fall-off prevention portion 113 is sufficiently larger than a size (diameter) of the nanopore 101. For example, the size of the fall-off prevention portion 113 relative to the diameter of the nanopore 101 may be any size as long as the progress of the biomolecule 109 can be stopped, and is desirably, for example, about 1.2 to 50 times. More specifically, in a case where single-stranded DNA is measured as the biomolecule 109, the diameter thereof is about 1.5 nm. Therefore, when the diameter of the nanopore 101 is about 1.5 nm to 2.5 nm, streptavidin (a diameter is about 5 nm) can be used as the fall-off prevention portion 113. When streptavidin is bound to the terminus, biotin is bound to the terminus. For biotinylation of the terminus, a commercially available kit can be used. In addition, streptavidin is not particularly limited, and may be, for example, mutant streptavidin in which a mutation is introduced so that the binding site with biotin is one site.

The substrate 102 comprises a base material 120 and a thin film 121 formed on one main surface of the base material 120. The nanopore 101 is formed in the thin film 121. In addition, although not illustrated, a substrate 203 may have an insulating layer. The base material 120 can be formed of a material of an electrical insulator, for example, an inorganic material and an organic material (including a polymer material). Examples of the electrically insulating material constituting the base material 120 include silicon, a silicon compound, glass, quartz, polydimethylsiloxane (PDMS), polytetrafluoroethylene (PTFE), polystyrene, and polypropylene. An example of the silicon compound includes silicon oxynitride such as silicon nitride, silicon oxide, or silicon carbide. In particular, the base material 120 can be formed of any of these materials, and may be, for example, silicon or a silicon compound. The nanopore 101 may be a lipid bilayer (biopore) implemented by an amphiphilic molecular layer having a pore in the center and a protein embedded therein.

A size and thickness of the substrate 102 are not particularly limited as long as the nanopore 101 can be provided. The substrate 102 can be produced by a method known in the art, or can be obtained from a commercially available product. For example, the substrate 102 can be produced using techniques such as photolithography or electron beam lithography, etching, laser vibration, injection molding, casting, molecular beam epitaxy, chemical vapor deposition (CVD), dielectric breakdown, electron beams, and focused ion beams. The substrate 102 may be coated in order to avoid adsorption of non-targeted molecules to the surface.

The substrate 102 has at least one nanopore 101. The nanopore 101 is specifically provided on the thin film 121, and may be optionally provided on the thin film 121 and the base material 120. Here, the “nanopore” and the “pore” are through-holes having a nanometer (nm) size (that is, a diameter of 1 nm or more and less than 1 μm), and are holes penetrating the substrate 102 to communicate the first liquid tank 104A and the second liquid tank 104B.

It is preferable that the substrate 102 comprises the thin film 121 for providing the nanopore 101. That is, the nanopore 101 can be simply and efficiently provided in the substrate 102 by forming the thin film 121 with a material and thickness suitable for forming a nano-sized pore on the substrate 120. From the viewpoint of ease of formation of the nanopore 101, the material of the thin film 121 is preferably, for example, silicon oxide (SiO₂), silicon nitride (SiN), silicon oxynitride (SiON), metal oxide, metal silicate, molybdenum disulfide (MoS₂), graphene, and the like. The thickness of the thin film 121 is 1 Å (angstrom) to 200 nm, preferably 1 Å to 100 nm, more preferably 1 Å to 50 nm, for example, about 5 nm. In addition, the thin film 121 (and may be the entire substrate 102) may be substantially transparent. Here, “substantially transparent” means that external light can be transmitted through approximately 50% or more and preferably 80% or more. In addition, the thin film may be a single layer or a multilayer.

It is also preferable to provide an insulating layer on the thin film 121. A thickness of the insulating layer is preferably 5 nm to 50 nm. Any insulating material can be used for the insulating layer, and it is preferable to use, for example, silicon or a silicon compound (silicon nitride, silicon oxide, or the like).

As the size of the nanopore 101, an appropriate size can be selected according to the type of biopolymer to be analyzed. The nanopore may have a uniform diameter, or may have different diameters at different sites. In the nanopore provided in the thin film 121 of the substrate 102, the smallest diameter portion, that is, the smallest diameter of the nanopore 101 is a diameter of 100 nm or less, for example, 0.9 nm to 100 nm, preferably 0.9 nm to 50 nm, for example, 0.9 nm to 10 nm, and specifically, preferably 1 nm or more and 5 nm or less, 3 nm or more and 5 nm or less, or the like. The nanopore 101 may be connected to a pore formed in the base material 120 and having a diameter of 1 μm or more.

In addition, in a case where the biomolecule to be analyzed is single-stranded nucleic acid (DNA), since a diameter of the single-stranded DNA is approximately 1.4 nm, the diameter of the nanopore 101 is preferably about 1.4 nm to 10 nm and more preferably about 1.4 nm to 2.5 nm, and specifically, can be about 1.6 nm. In a case where the biomolecule to be analyzed is double-stranded nucleic acid (DNA), since a diameter of the double-stranded DNA is approximately 2.6 nm, the diameter of the nanopore 101 is preferably about 3 nm to 10 nm and more preferably about 3 nm to 5 nm. Furthermore, the diameter of the nanopore 101 can be appropriately set according to an outer diameter dimension of the biopolymer to be analyzed (for example, a protein, a polypeptide, a sugar chain, or the like).

A depth (length) of the nanopore 101 can be adjusted by adjusting the entire thickness of the thin film 121 or the substrate 102. The depth of the nanopore 101 is preferably equal to a length of a monomer unit constituting the biomolecule to be analyzed. For example, in a case where a nucleic acid is selected as a biomolecule to be analyzed, the depth of the nanopore 101 is preferably about a single base, for example, about 0.3 nm. On the other hand, the depth of the nanopore can be 2 times or more, 3 times or more, or 5 times or more the size of the monomer unit constituting the biomolecule. For example, in a case where the biomolecule is composed of a nucleic acid, the depth of the nanopore can be analyzed when the depth of the nanopore is three or more bases, for example, about 1 nm or more. Therefore, it is possible to perform highly accurate analysis while maintaining robustness of the nanopore. In addition, a shape of the nanopore is basically circular, and may be elliptical or polygonal.

Furthermore, at least one nanopore 101 can be provided on the substrate 102, and in a case where a plurality of nanopores 101 are provided, the nanopores 101 may be arranged regularly or randomly. The nanopore 101 can be formed by a method known in the art, for example, by irradiation with an electron beam of a transmission electron microscope (TEM), using a nanolithography technique, an ion beam lithography technique, or the like.

Although the device illustrated in FIG. 1 has one nanopore 101 between the pair of liquid tanks 104A and 104B, this is merely an example, and the device can also have a plurality of nanopores 101 formed between the pair of liquid tanks 104A and 104B. In addition, as another example, it is also possible to provide an array device in which a plurality of nanopores 101 are formed on the substrate 102 and regions of the plurality of nanopores 101 are separated from each other by partition walls. In the array device, the first liquid tank 104A can be a common tank, and the second liquid tank 104B can be a plurality of individual tanks. In this case, an electrode can be arranged in each of the common tank and the individual tank.

In a case of an array type device comprising a plurality of thin films having nanopores, it is preferable to regularly arrange the thin films having nanopores. An interval for arranging the plurality of thin films can be 0.1 μm to 10 μm, and preferably 0.5 μm to 4 μm, depending on the performance of the electrode and the electric measurement system to be used.

A method of forming a nanopore in a thin film is not particularly limited, and for example, electron beam irradiation with a transmission electron microscope or dielectric breakdown due to voltage application can be used. For example, the method described in “Itaru Yanagi et al., Sci. Rep. 4, 5000 (2014)” can be used.

On the other hand, the first electrode 105A and the second electrode 105B are not particularly limited, and can be formed of, for example, a platinum group such as platinum, palladium, rhodium, and ruthenium, gold, silver, copper, aluminum, nickel, or the like; or graphite, for example, graphene (may be either a single layer or a multilayer), tungsten, tantalum, or the like.

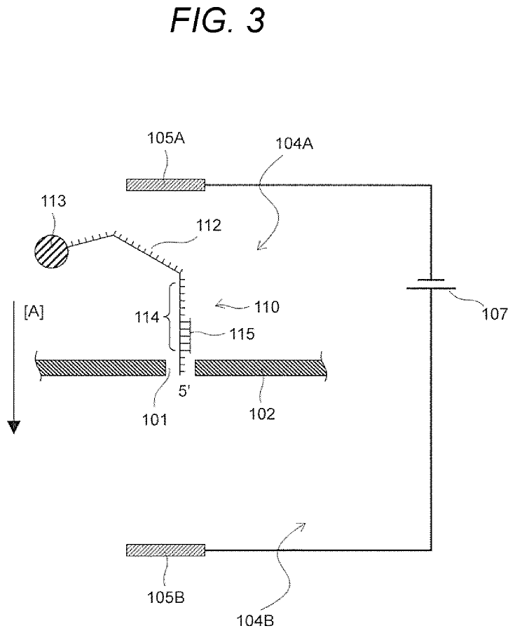

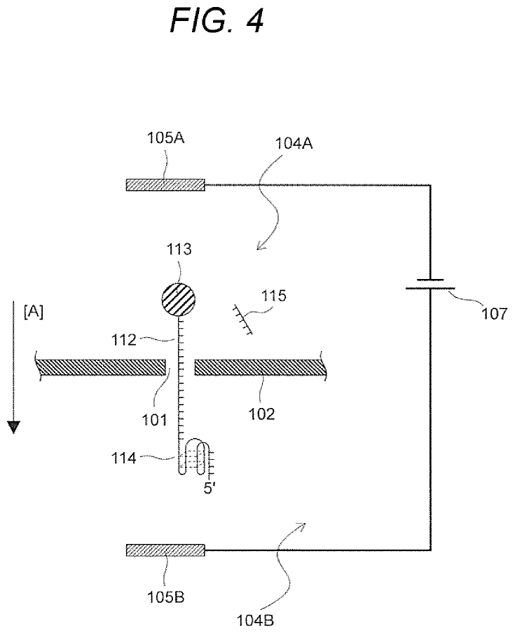

In the biomolecule analyzer configured as described above, in a state where the first liquid tank 104A is filled with the electrolyte solution 103 containing the biomolecule-adapter molecule complex 112, when a voltage is applied between the first electrode 105A and the second electrode 105B to generate a potential gradient in which the first liquid tank 104A is set to a negative potential or a ground potential and the second liquid tank 104B is set to a positive potential, as illustrated in FIG. 3, the terminus (5′-terminus) of the first adapter 110 is convayed in a direction of the nanopore 101 (a direction of the arrow A in FIG. 3). As illustrated in FIG. 4, the biomolecule-adapter molecule complex 112 is transferred to the second liquid tank 104B (a direction of the arrow A in FIG. 4) (passing) through the nanopore 101 due to the potential gradient generated between the first liquid tank 104A and the second liquid tank 104B. When the state transitions from the state in FIG. 3 to the state in FIG. 4, the three-dimensional structure formation inhibiting oligomer 115 hybridizing with the three-dimensional structure formation domain 114 cannot pass through the nanopore 101 and is peeled off (unzipped). As a result, the three-dimensional structure formation domain 114 introduced in the second liquid tank 104B forms a three-dimensional structure (G-quadruplex structure in the example in FIG. 4).

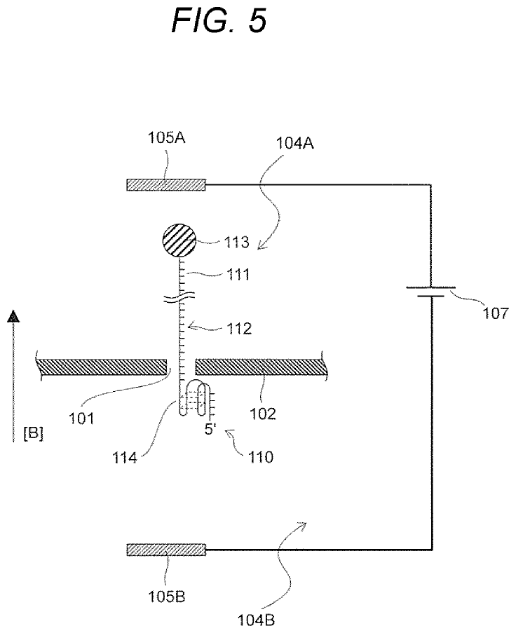

In addition, the biomolecule analyzer convays the biomolecule-adapter molecule complex 112 in which a three-dimensional structure is formed in the first adapter 110 from the first liquid tank 104A to the second liquid tank 104B through the nanopore 101, and the biomolecule-adapter molecule complex 112 can be convayed from the second liquid tank 104B to the first liquid tank 104A through the nanopore 101 by reversing a voltage gradient as illustrated in FIG. 5 (a direction of the arrow B in FIG. 5). That is, as illustrated in FIG. 4, the biomolecule-adapter molecule complex 112 can be convayed in a direction indicated by the arrow [A] in the drawing by the voltage gradient at which the first liquid tank 104A is set to a negative potential or a ground potential and the second liquid tank 104B is set to a positive potential. On the contrary, as illustrated in FIG. 5, the biomolecule-adapter molecule complex 112 can be convayed in a direction indicated by the arrow [B] in the drawing by the voltage gradient at which the second liquid tank 104B is set to a negative potential or a ground potential and the first liquid tank 104A is set to a positive potential. As such, the biomolecule analyzer can reciprocate the biomolecule-adapter molecule complex 112 in which a three-dimensional structure is formed in the first adapter 110 between the first liquid tank 104A and the second liquid tank 104B. At this time, since the three-dimensional structure is formed in the first adapter 110, when the biomolecule-adapter molecule complex 112 is convayed in the direction of the arrow B in FIG. 5, it is possible to prevent the biomolecule-adapter molecule complex 112 from falling off from the nanopore 101 due to the three-dimensional structure.

The voltage gradient formed between the first liquid tank 104A and the second liquid tank 104B may be set to either a positive potential, or a negative potential or a ground potential in order to convay negatively charged nucleic acid molecules. In the following description, in a case where one of the first liquid tank 104A and the second liquid tank 104B is set to a positive potential and the other one is set to a negative potential, the liquid tank to which the negative potential is set may be set to a ground potential.

In addition, in the biomolecule analyzer of FIG. 1, the measurement unit 106 can measure the ion current (blocking signal) flowing between the pair of electrodes 105A and 105B, and the computer 108 can acquire the sequence information of the biomolecule-adapter molecule complex 112 based on a value of the measured ion current (blocking signal). Although not illustrated in FIG. 1, the electrode can be provided in the nanopore 101, such that a tunnel current can be acquired and the sequence information can thus be acquired based on the tunnel current, or a change in characteristics of a transistor is detected to aquire the sequence information of the biomolecule 109.

Here, a method for determining base sequence information will be described in more detail. There are four types of ATGC in the base, and when these bases pass through the nanopore 101, a unique value of ion current (blocking current) is observed for each type. Therefore, an ion current at the time of passing through the nanopore 101 is measured in advance using a known sequence, and a current value corresponding to the known sequence is stored in a memory of the computer 108. Then, the types of bases constituting a bio-adapter molecule complex 111 to be analyzed can be sequentially determined by comparing the current value measured when the bases constituting the bio-adapter molecule complex 111 to be analyzed sequentially pass through the nanopore 101 with the current value corresponding to the known sequence stored in the memory. Here, the known sequence in which the ion current is measured in advance can be the number of bases (for example, a 2 base sequence, a 3 base sequence, or a 5 base sequence) corresponding to the depth (length) of the nanopore 101.

In addition, as a method for determining the base sequence of the biomolecule 109, the biomolecule 109 may be labeled with a fluorescent substance to be excited in the vicinity of the nanopore 101, and the emitted fluorescence may be detected. Furthermore, it is also possible to apply the method for determining the base sequence of the biomolecule 109 based on hybridization described in Reference Literature 1 (NANO LETTERS (2005), Vol. 5, pp. 421-424).

According to the method for determining base sequence information described above, it is possible to acquire the base sequence information of the biomolecule 109 when the biomolecule-adapter molecule complex 112 is convayed from the first liquid tank 104A to the second liquid tank 104B through the nanopore 101 from the state illustrated in FIG. 4 to the state illustrated in FIG. 5. In addition, when the biomolecule-adapter molecule complex 112 reciprocates between the first liquid tank 104A and the second liquid tank 104B through the nanopore 101, the base sequence information of the biomolecule 109 can be acquired.

When the biomolecule-adapter molecule complex 111 is reciprocated, the base sequence information of the biomolecule 109 may be acquired only when the biomolecule is convayed in the direction of the arrow [A] in FIG. 4, the base sequence information of the biomolecule 109 may be acquired only when the biomolecule is convayed in the direction of the arrow [B] in FIG. 5, or the base sequence information of the biomolecule 109 may be acquired in both the direction of the arrow [A] in FIG. 4 and the direction of the arrow [B] in FIG. 5. When the biomolecule 109 is convayed in the direction of the arrow [A] in FIG. 4, the base sequence information is determined from the 5′-terminus of the biomolecule 109 toward the 3′-terminus of the biomolecule 109, and when the biomolecule 109 is convayed in the direction of the arrow [B] in FIG. 5, the base sequence information is determined from the 3′-terminus of the biomolecule 109 toward the 5′-terminus of the biomolecule 109. In any case, a plurality of sets of base sequence information of the biomolecule 109 can be obtained, and the accuracy of the base sequence information can be improved. In other words, by reciprocating the biomolecule-adapter molecule complex 111, the base sequence of the biomolecule 109 can be read a plurality of times, and the reading accuracy can be improved.

In addition, an example of switching of the applied voltage during the reciprocating motion described above can include a method of automatically switching the applied voltage during a certain period of time. In this case, the voltage switching timing is programmed in the computer 108, and the voltage source 107 is controlled according to the program, such that the applied voltage can be switched at the timing to perform the reciprocating motion as described above.

Alternatively, the applied voltage can be switched using the base sequence information read during the reciprocating motion described above. For example, there is a method of incorporating a characteristic sequence in the first adapter molecule 110 or a region that generates a blocking current different from the base (AGCT), and switching the voltage at the timing of reading the characteristic sequence or a signal of the region. An example of the region that generates a blocking current different from a base can include a region containing a pseudo nucleic acid such as a peptide nucleic acid or an artificial nucleic acid. By reading the characteristic sequence or the signal of the region that generates the blocking current different from the base, it is possible to recognize that the reading of the base sequence of the biomolecule 109 is completed and the end of the biomolecule-adapter molecule complex 112 is close to the nanopore 101. Therefore, by switching the applied voltage at this timing, the biomolecule-adapter molecule complex 112 can be convayed in the opposite direction before the end of the biomolecule-adapter molecule complex 112 comes into contact with the nanopore 101. In particular, in the second liquid tank 104B, a three-dimensional structure is formed in the vicinity of the terminus of the biomolecule-adapter molecule complex 112. Therefore, it is possible to reliably prevent the biomolecule-adapter molecule complex 112 from falling off from the nanopore 101 when the biomolecule-adapter molecule complex 112 is convayed in the direction of the arrow B in FIG. 5. Therefore, the base sequence of the biomolecule 109 can be read a plurality of times according to the reciprocating motion described above, and the reading accuracy can be reliably improved.

As described above, it is possible to reliably reciprocate the biomolecule-adapter molecule complex 112 between the first liquid tank 104A and the second liquid tank 104B due to the voltage gradient generated between the first liquid tank 104A and the second liquid tank 104B by using the first adapter molecule 110. In the example described above, the double-stranded nucleic acid (DNA or RNA) is exemplified as the biomolecule 109, and even a protein (peptide chain) or a sugar chain can be analyzed as the biomolecule 109 according to a similar principle.

In the above description, as illustrated in FIGS. 3 to 5, the biomolecule-adapter molecule complex 112 is reciprocated by controlling the voltage gradient generated between the first liquid tank 104A and the second liquid tank 104B, and the convayance control of the biomolecule-adapter molecule complex 112 is not limited to this method. The biomolecule-adapter molecule complex 112 can be convayed between the first liquid tank 104A and the second liquid tank 104B using a so-called molecular motor. Here, the molecular motor refers to a protein molecule capable of moving on the biomolecule-adapter molecule complex 112. The molecular motor having such a function is not particularly limited, and examples thereof can include DNA polymerase, RNA polymerase, ribosome, and helicase. In particular, in the present embodiment, it is preferable to use, as the molecular motor, DNA polymerase that synthesizes a complementary strand from the 5′-terminus toward the 3′-terminus using single-stranded DNA as a template.

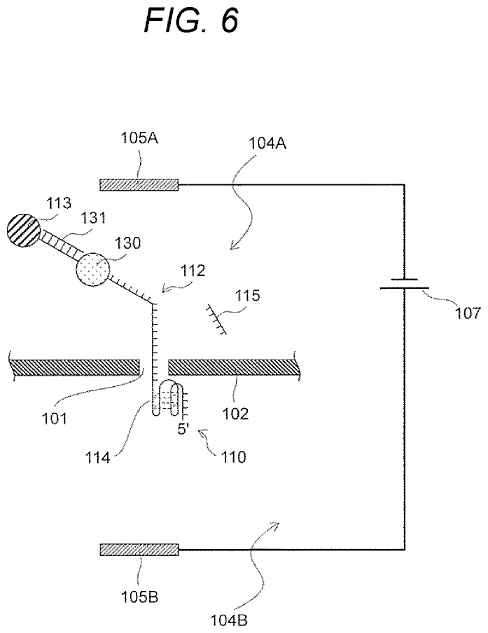

Specifically, as illustrated in FIG. 6, when a molecular motor 130 and a primer 131 are present in the first liquid tank 104A containing the biomolecule-adapter molecule complex 112, the primer 131 hybridizes with the second adapter molecule 111, and the molecular motor 130 is bound downstream thereof. In other words, the primer 131 is designed to hybridize with the second adapter molecule 111. Here, the primer 131 is not particularly limited, and an example thereof can include a single-stranded nucleotide having a base length of 5 to 40, preferably 15 to 35, and more preferably 18 to 25.

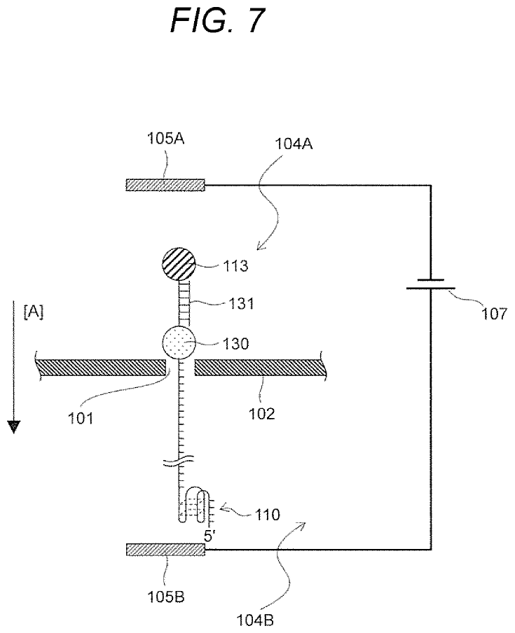

Next, as illustrated in FIG. 7, the biomolecule-adapter molecule complex 112 is convayed in the direction of the arrow A due to the voltage gradient generated between the first liquid tank 104A and the second liquid tank 104B, and the molecular motor 130 reaches the nanopore 101. Here, since a dimension Dm of the molecular motor 130 is larger than a diameter Dn of the nanopore 101 (Dm>Dn), when the molecular motor 130 reaches an inlet of the nanopore 101 (a side of the first liquid tank 104A), the molecular motor 130 cannot pass through the nanopore 101 and proceed to an outlet (a side of the second liquid tank 104B), and the molecular motor 130 stops at the inlet of the nanopore 101.

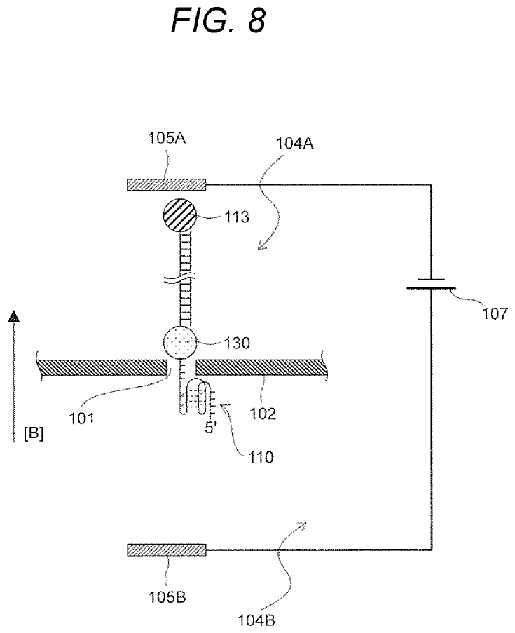

As illustrated in FIG. 8, the molecular motor 130 starts a complementary strand synthesis reaction in the direction from the 5′-terminus to the 3′-terminus starting from the 3′-terminus of the primer 131. When the complementary strand synthesis reaction by the molecular motor 130 proceeds, a force by which the biomolecule-adapter molecule complex 112 is pulled up by the molecular motor 130 is stronger than a force by which the biomolecule-adapter molecule complex 112 is convayed to the second liquid tank 104B due to the potential gradient. Therefore, the biomolecule-adapter molecule complex 112 is conveyed in the direction of the first liquid tank 104A (the direction of the arrow B in FIG. 8) against the potential gradient. At this time, as described above, the base sequence information of the biomolecule-adapter molecule complex 112 passing through the nanopore 101 can be acquired.

By controlling the conveyance of the biomolecule-adapter molecule complex 112 using the molecular motor 130, the nanopore passing speed can be set to 100 μs or more per base, and the blocking current derived from each base can be sufficiently measured.

As illustrated in FIGS. 6 to 8, even in a method for controlling the conveyance of the biomolecule-adapter molecule complex 112 using the molecular motor 130, since a three-dimensional structure is formed in the vicinity of the end of the biomolecule-adapter molecule complex 112. Therefore, it is possible to reliably prevent the biomolecule-adapter molecule complex 112 from falling off from the nanopore 101 when the biomolecule-adapter molecule complex 112 is transferred in the direction of the arrow B in FIG. 8.

### Embodiment 1-2

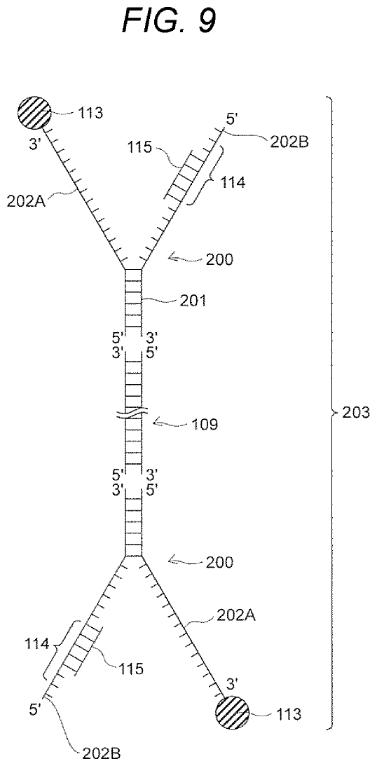

In the present embodiment, an adapter molecule 200 as illustrated in FIG. 9, which is different from each of the first adapter molecule 110 and the second adapter molecule 111 illustrated in FIG. 1 and so on, will be described. In the adapter molecule 200 exemplarily illustrated in FIG. 9 and the biomolecule analyzer using the same, the same components as those of the first adapter molecule 110 and the second adapter molecule 111 illustrated in FIG. 1 and the like are denoted by the same reference numerals, and a detailed description thereof will be omitted in this section.

The adapter molecule 200 illustrated in FIG. 9 comprises a double-stranded nucleic acid region 201 that directly binds to the biomolecule 109, and a pair of single-stranded nucleic acid regions 202A and 202B that are linked to an end different from the end bound to the biomolecule 109 in the double-stranded nucleic acid region 201 and consist of base sequences not complementary to each other. The single-stranded nucleic acid region 202A has a fall-off prevention portion 113 bound to the 3′-terminus, and the single-stranded nucleic acid region 202B has the 5′-terminus. In addition, the adapter molecule 200 illustrated in FIG. 9 comprises a three-dimensional structure formation domain 114 in the single-stranded nucleic acid region 202B. Furthermore, the adapter molecule 200 illustrated in FIG. 9 preferably comprises a three-dimensional structure formation inhibiting oligomer 115 hybridizing with the three-dimensional structure formation domain 114. In the example illustrated in FIG. 9, the fall-off prevention portion 113 is arranged at the end of the single-stranded nucleic acid region 202A having the 3′-terminus, and the three-dimensional structure formation domain 114 is arranged in the single-stranded nucleic acid region 202B. However, the fall-off prevention portion 113 may be arranged not at the end of the single-stranded nucleic acid region 202A, but at the end of the single-stranded nucleic acid region 202B having the 5′-terminus, and the three-dimensional structure formation domain 114 may be arranged in the single-stranded nucleic acid region 202A.

The biomolecule 109, the adapter molecule 200, and the DNA ligase are added to the electrolyte solution 103 filled in the first liquid tank 104A, such that a biomolecule-adapter molecule complex 203 can be formed in the electrolyte solution 103 filled in the first liquid tank 104A.

In addition, although not illustrated, the adapter molecule 200 and the biomolecule 109 may be indirectly bound to each other. The indirect linking means that the adapter molecule 200 and the biomolecule 109 are bound to each other via a nucleic acid fragment having a predetermined base length, and the adapter molecule 200 and the biomolecule 109 are bound to each other via a functional group introduced according to the type of the biomolecule 109.

Furthermore, it is preferable that the adapter molecule 200 has a 3′-protruding terminus (for example, the dT protruding terminus) as the end bound to the biomolecule 109 in the double-stranded nucleic acid region 201. By making the end the 3′-dT protruding terminus, it is possible to prevent formation of dimers of the adapter molecule 200 when the adapter molecule 200 and the biomolecule 109 are bound to each other.

Furthermore, in the adapter molecule 200, the length and base sequence of the double-stranded nucleic acid region 201 are not particularly limited, and can be any length and any base sequence. For example, the length of the double-stranded nucleic acid region 201 can be a base length of 5 to 100, 10 to 80, 15 to 60, or 20 to 40.

Furthermore, in the adapter molecule 200, the length and base sequence of each of the single-stranded nucleic acid regions 202A and 202B are not particularly limited, and can be any length and any base sequence. The single-stranded nucleic acid regions 202A and 202B may have the same lengths or different lengths. The single-stranded nucleic acid regions 202A and 202B may have a common base sequence or may have completely different base sequences as long as they are not complementary to each other. The “not complementary” means that a proportion of complementary sequences in the entire base sequences of the single-stranded nucleic acid regions 202A and 202B is 30% or less, preferably 20% or less, more preferably 10% or less, still more preferably 5% or less, and most preferably 3% or less.

A length of each of the single-stranded nucleic acid regions 202A and 202B can be, for example, a base length of 10 to 200, 20 to 150, 30 to 100, or 50 to 80. In addition, the single-stranded nucleic acid region 202B comprising the three-dimensional structure formation domain 114 can have a base sequence (for example, a base length of 20) consisting of 90% or more of thymine, preferably 100% of thymine, on the side of the 5′-terminus from the three-dimensional structure formation domain 114. When a proportion of the thymine in the base sequence on the side of the 5′-terminus from the dimensional structure formation domain 114 is within this range, formation of a higher order structure can be prevented, and the shape that can be easily introduced into the nanopore 101 can be obtained.

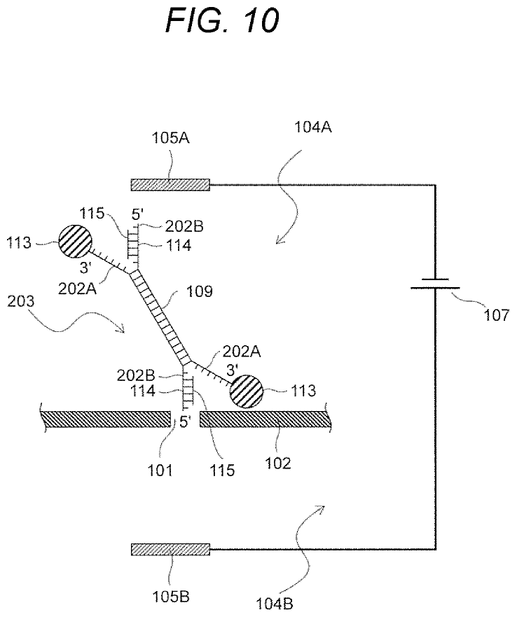

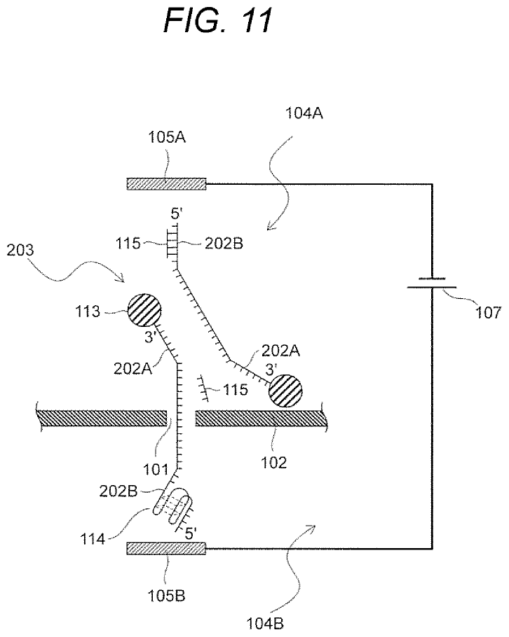

The biomolecule-adapter molecule complex 203 comprising the adapter molecule 200 illustrated in FIG. 9 and configured as described above can be analyzed by the biomolecule analyzer illustrated in FIG. 1. First, as illustrated in FIG. 10, in a state where the first liquid tank 104A is filled with the electrolyte solution 103 containing the biomolecule-adapter molecule complex 203, when a voltage is applied between the first electrode 105A and the second electrode 105B to generate a potential gradient in which the first liquid tank 104A is set to a negative potential and the second liquid tank 104B is set to a positive potential, the end of the single-stranded nucleic acid region 202B having no fall-off prevention portion 113 faces the inside of the nanopore 101. Furthermore, as illustrated in FIG. 11, the biomolecule-adapter molecule complex 203 is convayed to the second liquid tank 104B (passing) through the nanopore 101 due to the voltage gradient. When the state transitions from the state in FIG. 10 to the state in FIG. 11, the double-stranded nucleic acid (i.e., the double-stranded nucleic acid region 201; the biomolecule 109; and the three-dimensional structure formation domain 114 and the three-dimensional structure formation inhibiting oligomer 115 in the adapter molecule 200) in the biomolecule-adapter molecule complex 203 is peeled off (unzipped).

As such, it is possible to obtain a single-stranded nucleic acid that can pass through the nanopore 101 without performing a complicated denaturation treatment (for example, a heat treatment) on the biomolecule 109 that is a double-stranded nucleic acid by using the adapter molecule 200. That is, the double-stranded nucleic acid can be easily peeled off by using the adapter molecule 202. When the single-stranded nucleic acid region 202B having the three-dimensional structure formation domain 114 is introduced into the second liquid tank 104B, a three-dimensional structure is formed in the three-dimensional structure formation domain 114.

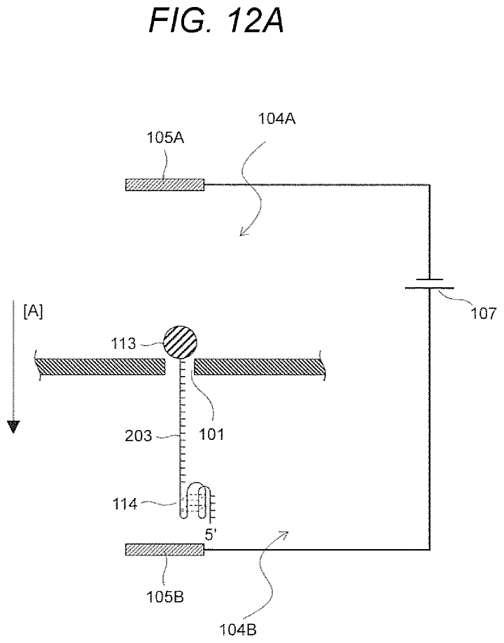

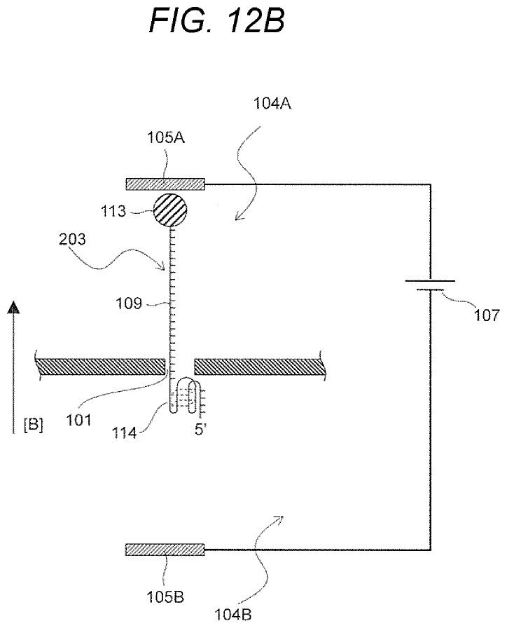

In addition, as illustrated in FIG. 11, the biomolecule analyzer convays the single-stranded biomolecule-adapter molecule complex 203 from the first liquid tank 104A to the second liquid tank 104B through the nanopore 101, and the biomolecule-adapter molecule complex 203 can be convayed from the second liquid tank 104B to the first liquid tank 104A through the nanopore 101 by reversing a voltage gradient. That is, as illustrated in FIG. 12A, the biomolecule-adapter molecule complex 203 can be convayed in the direction indicated by the arrow [A] in the figure by the voltage gradient at which the first liquid tank 104A is set to a negative potential and the second liquid tank 104B is set to a positive potential. On the contrary, as illustrated in FIG. 12B, the biomolecule-adapter molecule complex 203 can be convayed in a direction indicated by the arrow [B] in the figure by the voltage gradient at which the second liquid tank 104B is set to a negative potential and the first liquid tank 104A is set to a positive potential. As such, it is possible to reciprocate the single-stranded biomolecule-adapter molecule complex 203 between the first liquid tank 104A and the second liquid tank 104B by controlling the voltage gradient generated between the first liquid tank 104A and the second liquid tank 104B by the biomolecule analyzer.

In particular, in the second liquid tank 104B, a three-dimensional structure is formed in the vicinity of the terminus of the biomolecule-adapter molecule complex 203. Therefore, it is possible to reliably prevent the biomolecule-adapter molecule complex 203 from falling off from the nanopore 101 when the biomolecule-adapter molecule complex 203 is convayed in the direction of the arrow B in FIG. 12B. Therefore, the base sequence of the biomolecule 109 can be read a plurality of times according to the reciprocating motion described above, and the reading accuracy can be reliably improved.

### Embodiment 1-3

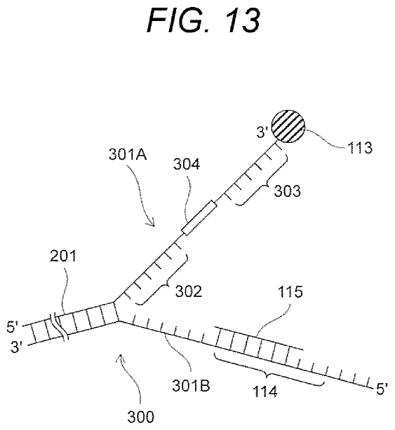

In the present embodiment, an adapter molecule 300 as illustrated in FIG. 13, which is different from each of the first adapter molecule 110, the second adapter molecule 111, and the adapter molecule 200 illustrated in FIGS. 1 and 9, will be described. In the adapter molecule 300 exemplarily illustrated in FIG. 13 and the biomolecule analyzer using the same, the same components as those of the first adapter molecule 110, the second adapter molecule 111, and the adapter molecule 200 illustrated in FIGS. 1 and 9 and the like are denoted by the same reference numerals, and a detailed description thereof will be omitted in this section.

The adapter molecule 300 illustrated in FIG. 13 comprises a double-stranded nucleic acid region 201 that directly binds to the biomolecule 109, a pair of single-stranded nucleic acid regions 301A and 301B that are bound to an end different from the end bound to the biomolecule 109 in the double-stranded nucleic acid region 201 and consist of base sequences not complementary to each other, and a fall-off prevention portion 113 arranged at the terminus of the single-stranded nucleic acid region 301A. The single-stranded nucleic acid region 301A has the 3′-terminus, and the single-stranded nucleic acid region 301B has the 5′-terminus. In addition, the adapter molecule 300 illustrated in FIG. 13 comprises a three-dimensional structure formation domain 114 in the single-stranded nucleic acid region 301B. Furthermore, the adapter molecule 300 illustrated in FIG. 13 preferably comprises a three-dimensional structure formation inhibiting oligomer 115 hybridizing with the three-dimensional structure formation domain 114.

The single-stranded nucleic acid region 301A in the adapter molecule 300 illustrated in FIG. 13 comprises a molecular motor binding portion 302 to which a molecular motor can bind. In addition, the single-stranded nucleic acid region 301A in the adapter molecule 300 illustrated in FIG. 13 comprises a primer binding portion 303 with which a primer can hybridize on a side of the 3′-terminus of the molecular motor binding portion 302. The primer binding portion 303 is not limited to a specific base sequence as long as it has a sequence complementary to the base sequence of the primer to be used. Here, the primer is not particularly limited, and an example thereof can include a single-stranded nucleotide having a base length of 5 to 40, preferably 15 to 35, and more preferably 18 to 25. Therefore, the primer binding portion 303 can be a region composed of a base sequence complementary to a base sequence having a base length of 10 to 40, preferably 15 to 35, and more preferably 18 to 25.

Furthermore, the single-stranded nucleic acid region 301A in the adapter molecule 300 illustrated in FIG. 13 comprises a spacer 304 between the molecular motor binding portion 302 and the primer binding portion 303. Here, the spacer 304 refers to a region to which the molecular motor cannot be bound, that is, a region containing no base composed of AGCT. The spacer 304 is not particularly limited, and can be a linear linker containing no base. In particular, a length of the spacer 304 is preferably a length corresponding to at least 2 bases, that is, about 0.6×2 nm or more. In other words, the spacer 304 allows the molecular motor binding portion 302 and the primer binding portion 303 to be separated from each other by 2 bases or more (about 0.6×2 nm or more). Examples of a material constituting the spacer 304 can include materials that can be arranged in a DNA chain, such as C3 Spacer, PC spacer, Spacer9, Spacer18, and dSpacer provided by Integrated DNA Technologies, Inc. In addition, a linear carbon chain, a linear amino acid, a linear fatty acid, a linear sugar chain, or the like can be used as the spacer 304.

Furthermore, in the adapter molecule 300 illustrated in FIG. 13, a predetermined region in the double-stranded nucleic acid region 201 can be a labeling sequence (not illustrated). The labeling sequence is also referred to as a barcode sequence or an index sequence, and refers to a base sequence unique to the adapter molecule 300. For example, the type of the adapter molecule 300 used can be specified based on the labeling sequence by preparing a plurality of adapter molecules 300 that differ only in the labeling sequence.

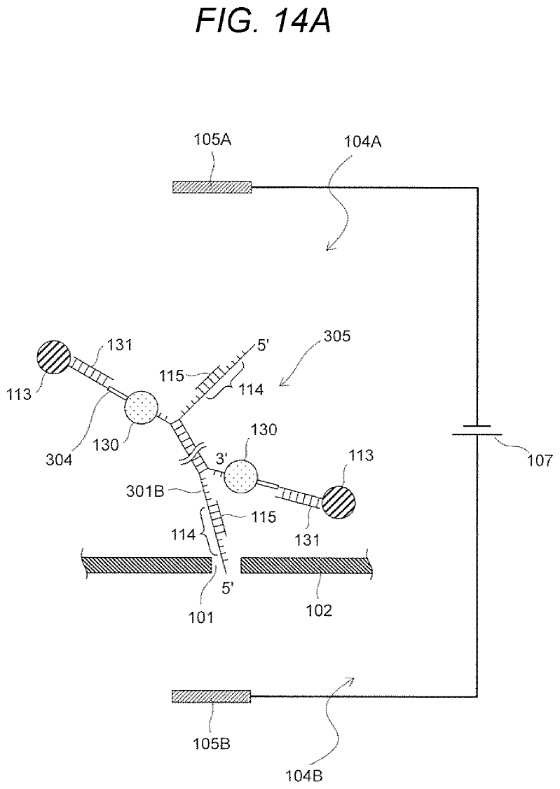

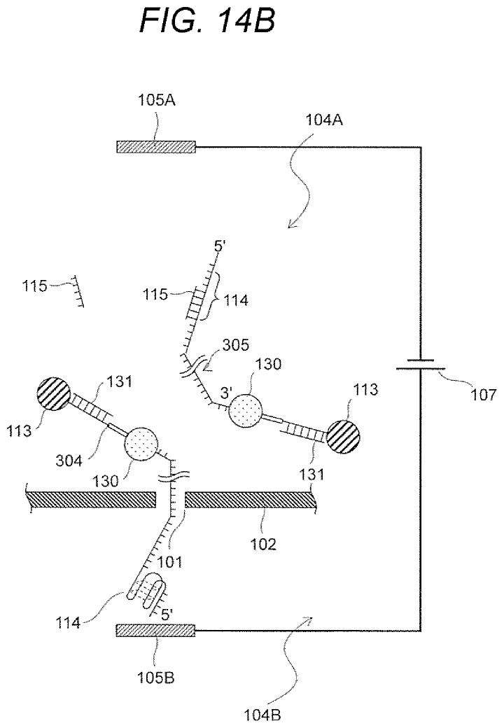

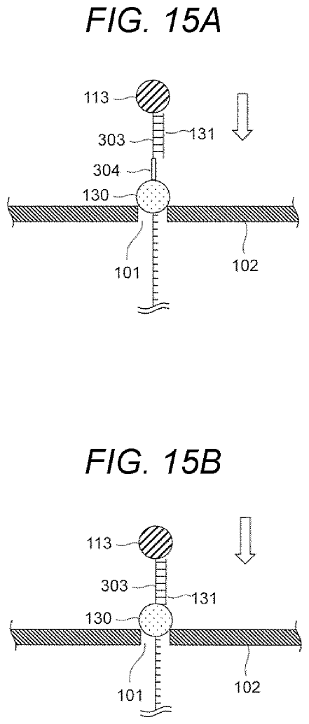

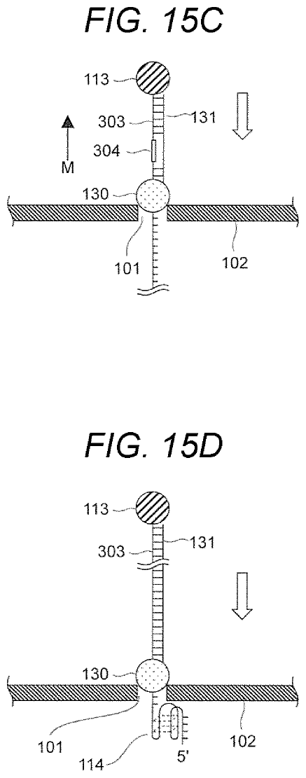

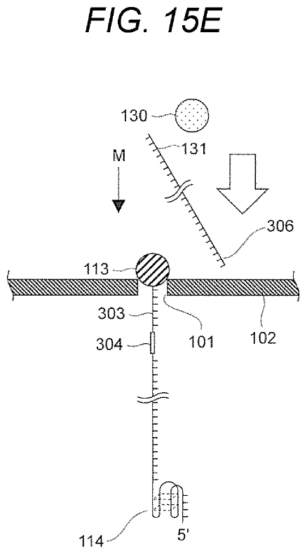

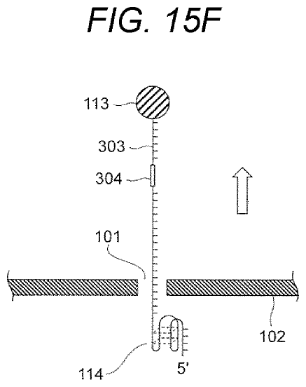

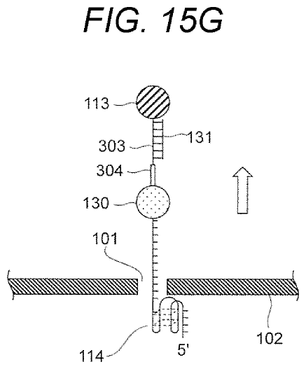

A method for analyzing the biomolecule 109 using the adapter molecule 300 configured as described above will be described with reference to FIGS. 14A and 14B and FIGS. 15A to 15G.

First, a biomolecule-adapter molecule complex 305 in which the adapter molecule 300 is bonded to each of both ends of the biomolecule 109 is prepared. The first liquid tank 104A is filled with an electrolyte solution containing the biomolecule-adapter molecule complex 305, the molecular motor 130, the primer 131, and the three-dimensional structure formation inhibiting oligomer 115. Accordingly, as illustrated in FIG. 14A, the molecular motor 130 binds to the molecular motor binding portion 302 in the adapter molecule 300, the primer 131 hybridizes with the primer binding portion 303, and the three-dimensional structure formation inhibiting oligomer 115 hybridizes with the three-dimensional structure formation domain 114 of the single-stranded nucleic acid region 301B.

Next, a voltage is applied between the first electrode 105A and the second electrode 105B to generate a potential gradient at which the first liquid tank 104A is set to a negative potential and the second liquid tank 104B is set to a positive potential. Therefore, the single-stranded nucleic acid region 301B is convayed in the direction of the nanopore 101, and the 5′-terminus region with which the three-dimensional structure formation inhibiting oligomer 115 does not hybridize is introduced into the nanopore 101. As illustrated in FIG. 14B, the biomolecule-adapter molecule complex 305 is convayed to the second liquid tank 104B (passing) through the nanopore 101 due to the potential gradient generated between the first liquid tank 104A and the second liquid tank 104B. In this case, the double-stranded nucleic acid (i.e., the double-stranded nucleic acid region 201; the biomolecule 109; and the three-dimensional structure formation inhibiting oligomer 115 and the three-dimensional structure formation domain 114 in the adapter molecule 300) in the biomolecule-adapter molecule complex 305 is peeled off (unzipped).

As such, it is possible to obtain a single-stranded nucleic acid that can pass through the nanopore 101 without performing a complicated denaturation treatment (for example, a heat treatment) on the biomolecule 109 that is a double-stranded nucleic acid even in a case where the adapter molecule 300 is used. That is, the double-stranded nucleic acid can be easily peeled off even in the case where the adapter molecule 300 is used. In the states illustrated in FIGS. 14A and 14B, since the primer 131 and the molecular motor 130 are separated by the length of the spacer 304, the complementary strand synthesis reaction by the molecular motor 130 starting from the 3′-terminus of the primer 131 is not started. When the single-stranded nucleic acid region 301B having the three-dimensional structure formation domain 114 is introduced into the second liquid tank 104B, a three-dimensional structure is formed in the three-dimensional structure formation domain 114.

As illustrated in FIG. 15A, the single-stranded biomolecule-adapter molecule complex 305 passes through the nanopore 101 due to the potential gradient generated between the first liquid tank 104A and the second liquid tank 104B, and then, the molecular motor 130 reaches the nanopore 101. Since the single-stranded biomolecule-adapter molecule complex 305 is negatively charged, the biomolecule-adapter molecule complex 305 further proceeds in a downstream direction, and a change in shape around the spacer 304 occurs. Then, the molecular motor 130 comes into contact with and binds to the 3′-terminus of the primer 131 (FIG. 15B). Accordingly, the molecular motor 130 starts a complementary strand synthesis reaction in the direction from the 3′-terminus from the 5′-terminus starting from the 3′-terminus of the primer 131. The void arrow in each of FIGS. 15A to 15H indicates the potential gradient from the negative electrode to the positive electrode.

As illustrated in FIG. 15C, when the complementary strand synthesis reaction by the molecular motor 130 proceeds, a force by which the single-stranded biomolecule-adapter molecule complex 305 is pulled up by the molecular motor 130 is stronger than a force by which the single-stranded biomolecule-adapter molecule complex 305 is conveyed to the second liquid tank 104B due to the potential gradient. Therefore, the single-stranded biomolecule-adapter molecule complex 305 is conveyed in the direction of the first liquid tank 104A (the direction of the arrow M in FIG. 15C) against the potential gradient. At this time, the base sequence information of the biomolecule-adapter molecule complex 305 passing through the nanopore 101 can be acquired.

As illustrated in FIG. 15D, when the three-dimensional structure formed in the single-stranded nucleic acid region 301B of the biomolecule-adapter molecule complex 305 reaches the nanopore 101, the conveyance operation by the molecular motor 130 and sequencing are stopped. The inside of the second liquid tank 104B is set to a stronger positive potential at the stage where the conveyance operation by the molecular motor 130 and sequencing are stopped. As a result, as illustrated in FIG. 15E, the biomolecule-adapter molecule complex 305 is convayed to the second liquid tank 104B due to the potential gradient (a direction of the arrow M in FIG. 15E). At this time, a complementary strand 306 of the biomolecule-adapter molecule complex 305 synthesized by the molecular motor 130 is peeled off (unzipped) from the biomolecule-adapter molecule complex 305, and the molecular motor 130 is separated from the biomolecule-adapter molecule complex 305.

The timing at which the inside of the second liquid tank 104B is set to a stronger positive potential can be a method of automatically switching a voltage at a certain time or a method of switching a voltage using read base sequence information. Alternatively, since a decrease in blocking current can be measured when the three-dimensional structure approaches the nanopore 101, the inside of the second liquid tank 104B may be set to a stronger positive potential when the decrease in blocking current is detected. In any of these methods, by forming a three-dimensional structure in the single-stranded nucleic acid region 301B, the entire single-stranded biomolecule-adapter molecule complex 305 can be prevented from passing through the nanopore 101.

Next, as illustrated in FIG. 15F, a voltage is applied between the first electrode 105A and the second electrode 105B is inverted to generate a potential gradient at which the first liquid tank 104A is set to a positive potential and the second liquid tank 104B is set to a negative potential. Accordingly, the single-stranded biomolecule-adapter molecule complex 305 can be convayed from the second liquid tank 104B toward the first liquid tank 104A through the nanopore 101.

Thereafter, as illustrated in FIG. 15G, the molecular motor 130 and the primer 131 are added to the electrolyte solution 103 filled in the first liquid tank 104A, and then, the primer 131 is allowed to hybridize with the primer binding portion 303, and the molecular motor 130 is bound to the molecular motor binding portion 302. Thereafter, the voltage applied between the first electrode 105A and the second electrode 105B is inverted again to generate a potential gradient at which the first liquid tank 104A is set to a negative potential and the second liquid tank 104B is set to a positive potential. Accordingly, the biomolecule-adapter molecule complex 305, with which the primer 131 hybridizes, to which the molecular motor 130 is bound is convayed in the direction of the second liquid tank 104B. As illustrated in FIG. 15B, a change in shape around the spacer 304 occurs, and thus, a state in which the molecular motor 130 is in contact with the 3′-terminus of the primer 131 is formed. That is, sequencing can be performed for each conveyance operation by the molecular motor 130 by repeating FIGS. 15A to 15G.

According to the reference document (Nat Nanotechnol. 2010. November; 5 (11): 798-806), it is suggested that measurement using the molecular motor 130 (the diameter of the nanopore 101: 1.4 nm) is performed while applying a voltage of at least 80 mV or higher. In this case, the reference document (Nature physics, 5, 347-351, 2009.) suggests that a force of approximately 24 pN is applied. Therefore, in the present embodiment, in a case where the fall-off prevention portion 113 is measured at a voltage of 80 mV, it is preferable that binding to the single-stranded nucleic acid region 301A is performed at a binding force of 24 pN or more.

In particular, in the second liquid tank 104B, a three-dimensional structure is formed in the vicinity of the terminus of the biomolecule-adapter molecule complex 305. Therefore, it is possible to reliably prevent the biomolecule-adapter molecule complex 305 from falling off from the nanopore 101 when the biomolecule-adapter molecule complex 305 is convayed in the direction of the direction of the first liquid tank 104A from the second liquid tank 104B. Therefore, the base sequence of the biomolecule 109 can be read a plurality of times according to the reciprocating motion described above, and the reading accuracy can be reliably improved.

### Embodiment 2-1

In the present embodiment, an adapter molecule having a plurality of primer binding sites and molecular motor binding portions corresponding to the primer binding sites, which is different from the adapter molecules described in the embodiments 1-1 to 1-3, will be described. In the adapter molecule and the like described in the present embodiment, the same configurations as those of the adapter molecules described in the embodiments 1-1 to 1-3 are denoted by the same reference numerals, and a detailed description thereof will be omitted in the present section.

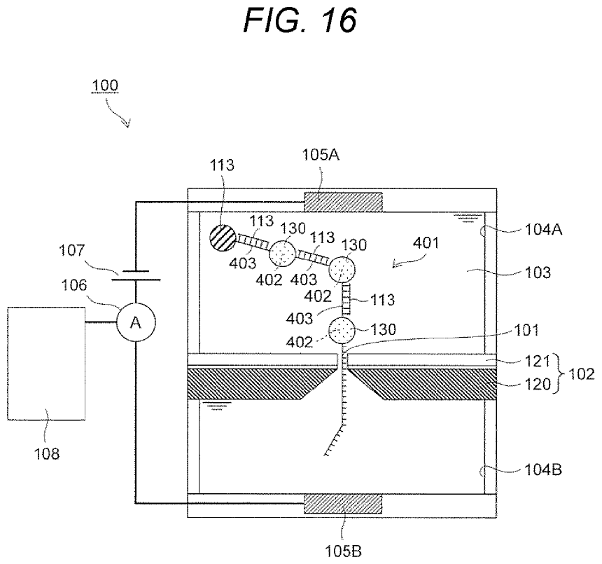

FIG. 16 illustrates a biomolecule analyzer 100 for analyzing a biomolecule-adapter molecule complex 401 comprising an adapter molecule 400 according to the present embodiment. The biomolecule analyzer 100 is a device that analyzes the biomolecule-adapter molecule complex 401, and is a device for biomolecule analysis that measures an ion current by a blocking current method. The biomolecule analyzer 100 comprises a substrate 102 in which a nanopore 101 is formed, a pair of liquid tanks 104 (a first liquid tank 104A and a second liquid tank 104B) arranged to be in contact with the substrate 102 with the substrate 102 interposed therebetween and filled with an electrolyte solution 103 therein, and a pair of electrodes 105 (a first electrode 105A and a second electrode 105B) in contact with the first liquid tank 104A and the second liquid tank 104B, respectively. At the time of measurement, a predetermined voltage is applied from a voltage source 107 between the pair of electrodes 105, and a current flows between the pair of electrodes 105. A magnitude of the current flowing between the electrodes 105 is measured by an ammeter 106, and a measured value thereof is analyzed by a computer 108.

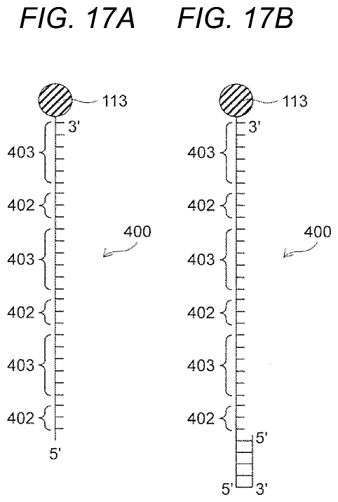

The adapter molecule 400 illustrated in the present embodiment includes a plurality of sets of a molecular motor binding portion 402 to which a molecular motor 130 can bind and a primer binding portion 403 with which a primer 131 can hybridize on a side of the 3′-terminus from the molecular motor binding portion 402, as illustrated in FIGS. 17(A) and 17(B). The adapter molecule 400 may consist of single-stranded DNA as illustrated in FIG. 17(A), or in a case where the biomolecule 109 to be analyzed is double-stranded DNA as illustrated in FIG. 17(B), the end linked to the biomolecule 109 may be double-stranded DNA. In addition, the adapter molecule 400 preferably includes a fall-off prevention portion 113 at one end portion (for example, the 3′-end terminus).

Here, the number of combinations of the molecular motor binding portion 402 and the primer binding site 403 is not particularly limited as long as it is plural (2 or more), can be, for example, 2 to 10 sets, and is more preferably 2 to 5 sets. The number of combinations of the molecular motor binding portion 402 and the primer binding site 403 corresponds to the number of times of reading the base sequence of the biomolecule 109. Therefore, the number of times of reading the base sequence of the biomolecule 109 is determined in advance, and the number of combinations of the molecular motor binding portion 402 and the primer binding site 403 can be set so as to correspond to the number of times.

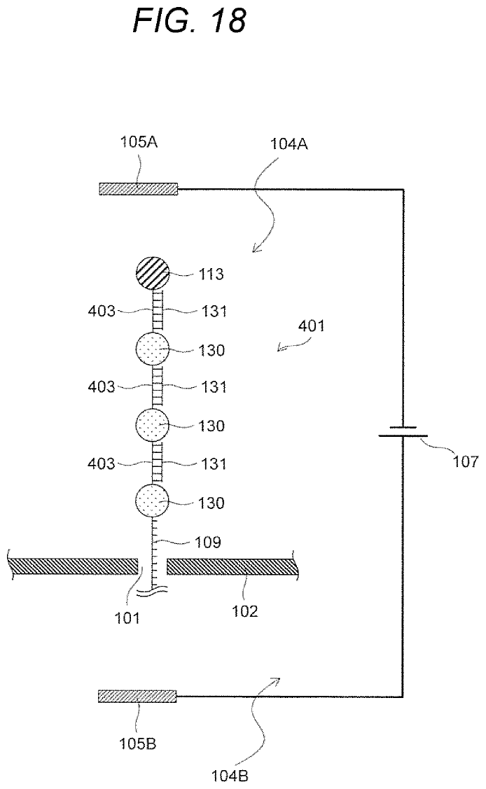

A method for analyzing the biomolecule 109 using the adapter molecule 400 configured as described above will be described with reference to FIGS. 18 to 21.

First, a biomolecule-adapter molecule complex 401 in which the adapter molecule 400 is bonded to one end of the biomolecule 109 is prepared. The first liquid tank 104A is filled with an electrolyte solution containing the biomolecule-adapter molecule complex 401, the molecular motor 130, and the primer 131. Accordingly, the molecular motor 130 is bound to each of the plurality of molecular motor binding portions 402 in the adapter molecule 400, and the primer 131 hybridizes with each of the plurality of primer binding portions 403.

Next, a voltage is applied between the first electrode 105A and the second electrode 105B to generate a potential gradient at which the first liquid tank 104A is set to a negative potential and the second liquid tank 104B is set to a positive potential. Accordingly, as illustrated in FIG. 18, the end to which the adapter molecule 400 is not bound in the biomolecule-adapter molecule complex 401 is convayed in the direction of the nanopore 101 to be introduced into the nanopore 101. The biomolecule-adapter molecule complex 401 is convayed to the second liquid tank 104B (passing) through the nanopore 101 due to the potential gradient generated between the first liquid tank 104A and the second liquid tank 104B. Although not illustrated, the fall-off prevention portion 113 is added to the electrolyte solution 103 in the second liquid tank 104B, such that the fall-off prevention portion 113 can be added to the end of the biomolecule-adapter molecule complex 401 that has convayed to the second liquid tank 104B.

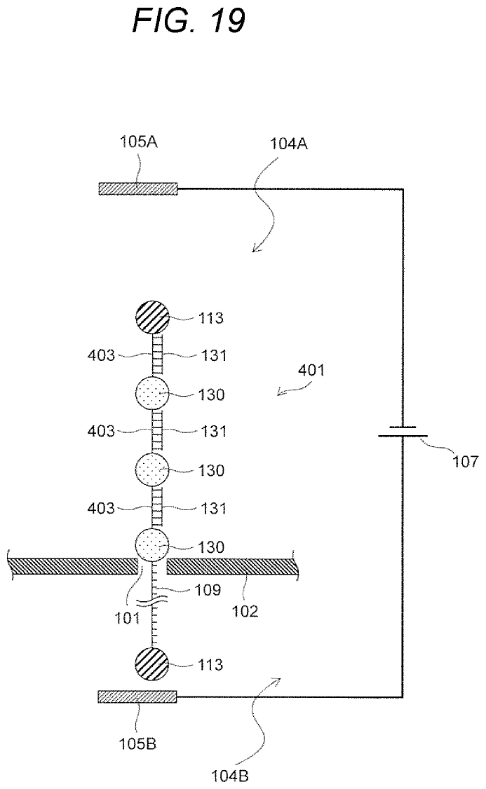

As illustrated in FIG. 19, the biomolecule-adapter molecule complex 401 passes through the nanopore 101 due to the potential gradient generated between the first liquid tank 104A and the second liquid tank 104B, and then, the molecular motor 130 located closest to the biomolecule 109 reaches the nanopore 101. In this state, the molecular motor 130 starts a complementary strand synthesis reaction in the direction from the 3′-terminus from the 5′-terminus starting from the 3′-terminus of the primer 131.

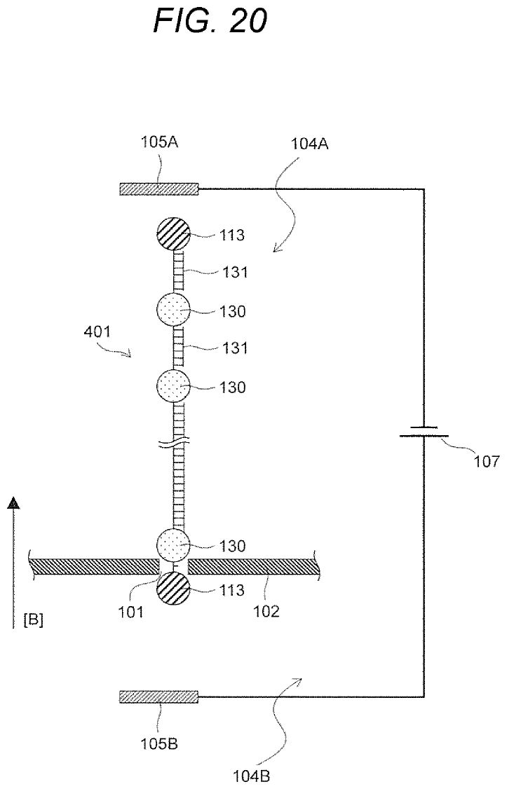

As illustrated in FIG. 20, when the complementary strand synthesis reaction by the molecular motor 130 proceeds, a force by which the biomolecule-adapter molecule complex 401 is pulled up by the molecular motor 130 is stronger than a force by which the biomolecule-adapter molecule complex 401 is convayed to the second liquid tank 104B due to the potential gradient. Therefore, the biomolecule-adapter molecule complex 401 is conveyed in the direction of the first liquid tank 104A (the direction of the arrow B in FIG. 20) against the potential gradient. At this time, the base sequence information of the biomolecule-adapter molecule complex 401 passing through the nanopore 101 can be acquired.

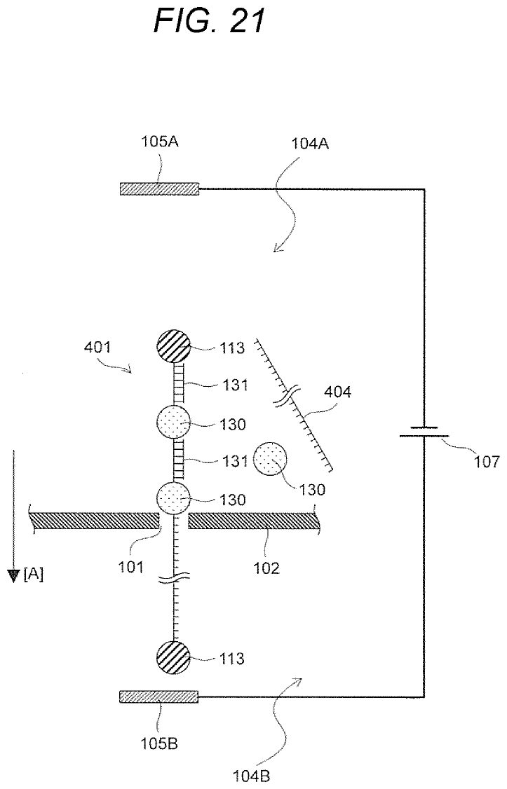

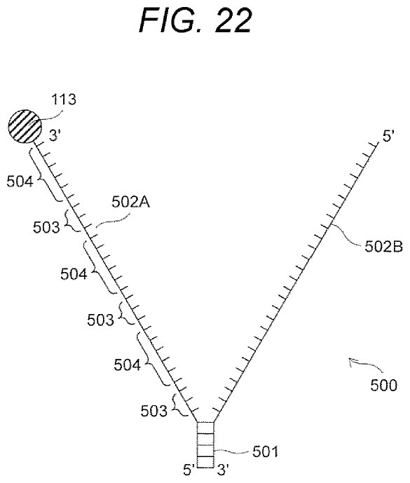

As illustrated in FIG. 20, when the fall-off prevention portion 113 bound to the end located in the second liquid tank 104B in the biomolecule-adapter molecule complex 401 reaches the nanopore 101, the conveyance operation by the molecular motor 130 and sequencing are stopped. The inside of the second liquid tank 104B is set to a stronger positive potential at the stage where the conveyance operation by the molecular motor 130 and sequencing are stopped. As a result, as illustrated in FIG. 21, the biomolecule-adapter molecule complex 401 is convayed to the second liquid tank 104B due to the potential gradient (a direction of the arrow A in FIG. 21). At this time, a complementary strand 404 of the biomolecule-adapter molecule complex 401 synthesized by the molecular motor 130 is peeled off (unzipped) from the biomolecule-adapter molecule complex 401, and the molecular motor 130 is separated from the biomolecule-adapter molecule complex 401.

The timing at which the inside of the second liquid tank 104B is set to a stronger positive potential can be a method of automatically switching a voltage at a certain time or a method of switching a voltage using read base sequence information. Alternatively, since a decrease in blocking current can be measured when the fall-off prevention portion 113 approaches the nanopore 101, the inside of the second liquid tank 104B may be set to a stronger positive potential when the decrease in blocking current is detected. In any of these methods, the fall-off prevention portion 113 can prevent the entire biomolecule-adapter molecule complex 401 from passing through the nanopore 101 and falling off.

After the complementary strand 404 and the molecular motor 130 are peeled off, as illustrated in FIG. 21, the next molecular motor 130 located closest to the biomolecule 109 reaches the nanopore 101. In this state, the molecular motor 130 starts a complementary strand synthesis reaction from the 3′-terminus of the primer 131. That is, as illustrated in FIG. 20, the biomolecule-adapter molecule complex 401 is conveyed again in the direction of the first liquid tank 104A against the potential gradient by the next molecular motor 130. At this time, the base sequence information of the biomolecule-adapter molecule complex 401 passing through the nanopore 101 can be acquired again.

As described above, the base sequence information can be acquired a plurality of times according to the number of sets of the molecular motor 130 and the primer 131 bound to the adapter molecule 400. In a case where the adapter molecule 400 is used, the base sequence information of the biomolecule 109 can be acquired a plurality of times by the series of processing described above without performing a step of controlling to invert the voltage applied between the first liquid tank 104A and the second liquid tank 104B or binding the molecular motor 130 and the primer 131 again after one measurement. That is, in a case where the adapter molecule 400 is used, the accuracy of reading the base sequence of the biomolecule 109 can be reliably improved according to the reciprocating motion by a significantly simple operation.

### Embodiment 2-2

In the present embodiment, an adapter molecule 500 illustrated in FIG. 22, which is different from the adapter molecule 400 illustrated in FIG. 16, will be described. In the adapter molecule 500, the same configurations as those of the adapter molecules described in the embodiments 1-1 to 1-3 or the adapter molecule 400 are denoted by the same reference numerals, and a detailed description thereof will be omitted in the present section.

The adapter molecule 500 illustrated in FIG. 22 comprises a double-stranded nucleic acid region 501 that directly binds to the biomolecule 109, and a pair of single-stranded nucleic acid regions 502A and 502B that are bound to an end different from the end binding to the biomolecule 109 in the double-stranded nucleic acid region 501 and consist of base sequences not complementary to each other. In addition, the adapter molecule 500 illustrated in FIG. 22 comprises a plurality of sets of a molecular motor binding portion 503 and a primer binding portion 504 in the single-stranded nucleic acid region 502A. The single-stranded nucleic acid region 502B has the 5′-terminus, and the single-stranded nucleic acid region 502A has the 3′-terminus. It is preferable to provide a fall-off prevention portion 113 at the terminus of the single-stranded nucleic acid region 502A.

In addition, a length and base sequence of the single-stranded nucleic acid region 502B are not particularly limited, and can be any length and any base sequence. The single-stranded nucleic acid regions 502A and 502B may have the same lengths or different lengths. Here, the “single-stranded nucleic acid regions 502A and 502B are not complementary to each other” means that a proportion of complementary sequences in the entire base sequences of the single-stranded nucleic acid regions 502A and 502B is 30% or less, preferably 20% or less, more preferably 10% or less, still more preferably 5% or less, and most preferably 3% or less.

The length of the single-stranded nucleic acid regions 502B can be, for example, a base length of 10 to 200, 20 to 150, 30 to 100, or 50 to 80. As an example, in particular, the single-stranded nucleic acid regions 502B having the 5′-terminus can consist of a base sequence consisting of 90% or more of thymine, and preferably a base sequence consisting of 100% of thymine. When a proportion of the thymine in the single-stranded nucleic acid region 502B having the 5′-terminus is within this range, formation of a higher order structure can be prevented, and the shape that can be easily introduced into the nanopore 101 can be maintained.

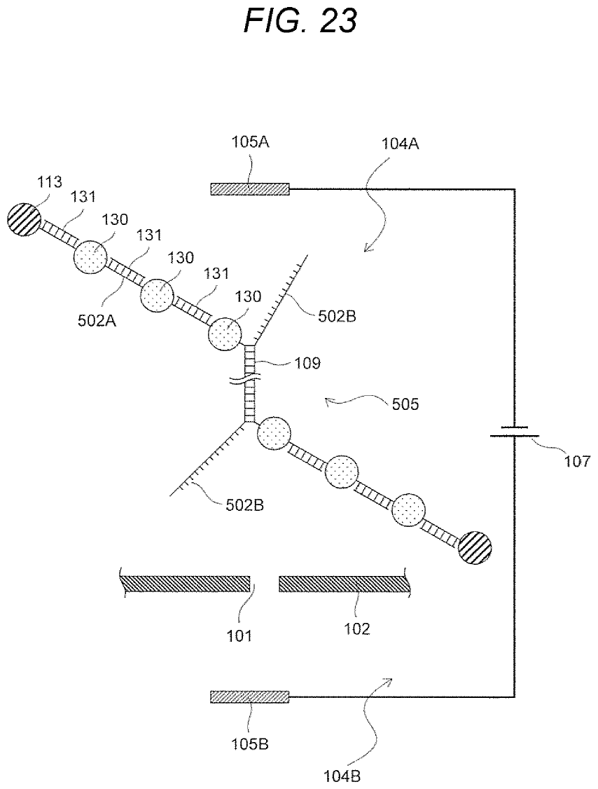

The biomolecule 109, the adapter molecule 500, and the DNA ligase are added to the electrolyte solution 103 filled in the first liquid tank 104A, such that a biomolecule-adapter molecule complex 505 may be formed in the electrolyte solution 103 filled in the first liquid tank 104A, as illustrated in FIG. 23.

In addition, although not illustrated, the adapter molecule 500 and the biomolecule 109 may be indirectly bound to each other. The indirect linking means that the adapter molecule 500 and the biomolecule 109 are linked to each other by a nucleic acid fragment having a predetermined base length, and the adapter molecule 500 and the biomolecule 109 are bound to each other by a functional group introduced according to the type of the biomolecule 109.

Furthermore, it is preferable that the adapter molecule 500 has a 3′-protruding terminus (for example, the dT protruding terminus) as the end linked to the biomolecule 109 in the double-stranded nucleic acid region 501. By making the end the 3′-dA protruding terminus, it is possible to prevent formation of dimers of the adapter molecule 500 when the adapter molecule 500 and the biomolecule 109 are linked to each other.

Furthermore, in the adapter molecule 500, the length and base sequence of the double-stranded nucleic acid region 501 are not particularly limited, and can be any length and any base sequence. For example, the length of the double-stranded nucleic acid region 501 can be a base length of 5 to 100, 10 to 80, 15 to 60, or 20 to 40.

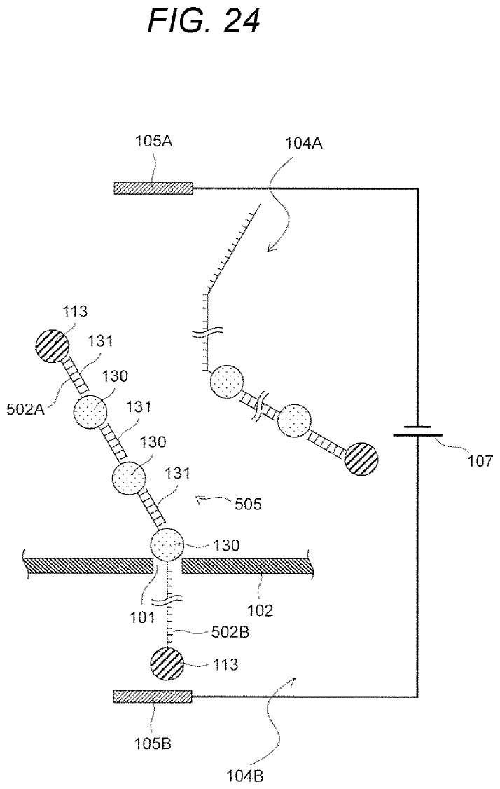

The biomolecule-adapter molecule complex 505 comprising the adapter molecule 500 illustrated in FIG. 22 and configured as described above can be analyzed by the biomolecule analyzer illustrated in FIG. 16. First, although not illustrated, the first liquid tank 104A is filled with an electrolyte solution 103 containing the biomolecule-adapter molecule complex 505, the molecular motor 130, and the primer 131. Accordingly, as illustrated in FIG. 23, in the first liquid tank 104A, a plurality of molecular motors 130 and primers 131 are bound to the biomolecule-adapter molecule complex 505. In this state, when a voltage is applied between the first electrode 105A and the second electrode 105B to generate a potential gradient at which the first liquid tank 104A is set to a negative potential and the second liquid tank 104B is set to a positive potential, the end (a single-stranded nucleic acid) of the single-stranded nucleic acid region 502B faces the inside of the nanopore 101. Furthermore, as illustrated in FIG. 24, the biomolecule-adapter molecule complex 505 is convayed to the second liquid tank 104B (passing) through the nanopore 101 due to the voltage gradient. When the state transitions from the state in FIG. 23 to the state in FIG. 24, the double-stranded nucleic acid (i.e., the double-stranded nucleic acid region 501 and the biomolecule 109 in the adapter molecule 500) in the biomolecule-adapter molecule complex 505 is peeled off (unzipped).

As such, it is possible to obtain a single-stranded nucleic acid that can pass through the nanopore 101 without performing a complicated denaturation treatment (for example, a heat treatment) on the biomolecule 109 that is a double-stranded nucleic acid by using the adapter molecule 500. That is, the double-stranded nucleic acid can be easily peeled off by using the adapter molecule 500. As illustrated in FIG. 24, the fall-off prevention portion 113 is added to the electrolyte solution 103 in the second liquid tank 104B, such that the fall-off prevention portion 113 can be added to the end of the biomolecule-adapter molecule complex 505 that has convayed to the second liquid tank 104B.

As illustrated in FIG. 24, the single-stranded biomolecule-adapter molecule complex 505 passes through the nanopore 101 due to the potential gradient generated between the first liquid tank 104A and the second liquid tank 104B, and then, the molecular motor 130 located closest to the biomolecule 109 reaches the nanopore 101. In this state, the molecular motor 130 starts a complementary strand synthesis reaction in the direction from the 3′-terminus from the 5′-terminus starting from the 3′-terminus of the primer 131.

When the complementary strand synthesis reaction by the molecular motor 130 proceeds, a force by which the biomolecule-adapter molecule complex 505 is pulled up by the molecular motor 130 is stronger than a force by which the single-stranded biomolecule-adapter molecule complex 505 is convayed to the second liquid tank 104B due to the potential gradient. Therefore, the biomolecule-adapter molecule complex 505 is conveyed in the direction of the first liquid tank 104A against the potential gradient. At this time, the base sequence information of the biomolecule-adapter molecule complex 505 passing through the nanopore 101 can be acquired.

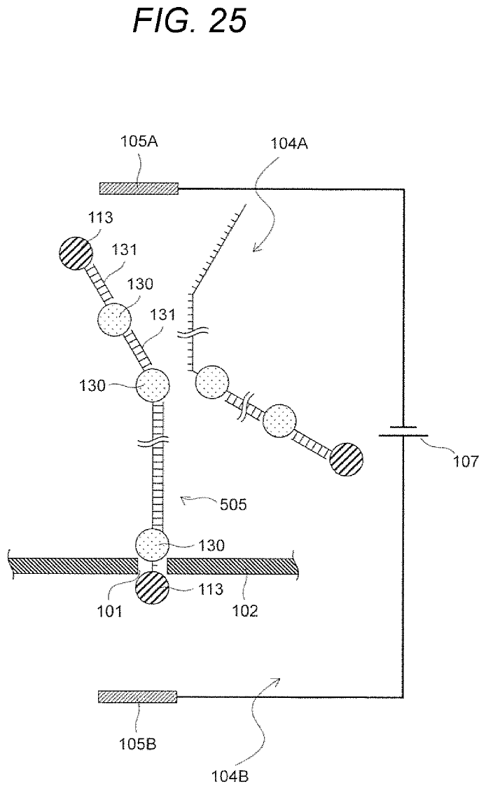

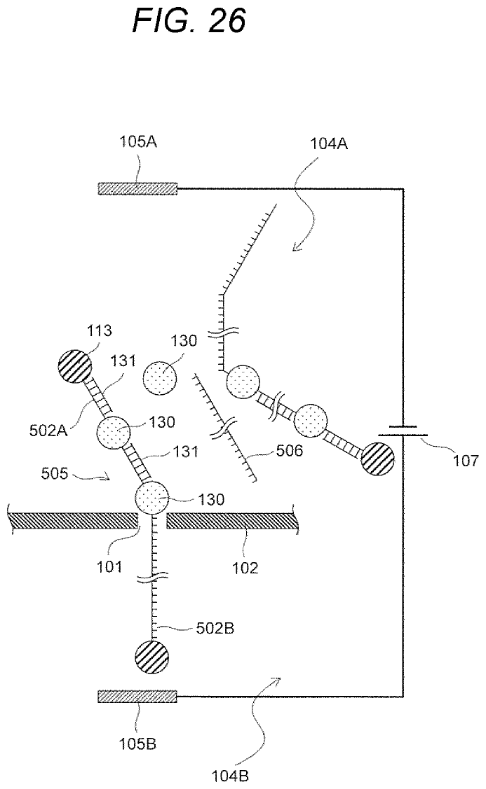

As illustrated in FIG. 25, when the fall-off prevention portion 113 bound to the end located in the second liquid tank 104B in the biomolecule-adapter molecule complex 505 reaches the nanopore 101, the conveyance operation by the molecular motor 130 and sequencing are stopped. The inside of the second liquid tank 104B is set to a stronger positive potential at the stage where the conveyance operation by the molecular motor 130 and sequencing are stopped. As a result, as illustrated in FIG. 26, the biomolecule-adapter molecule complex 50 is convayed to the second liquid tank 104B due to the potential gradient. At this time, a complementary strand 506 of the biomolecule-adapter molecule complex 505 synthesized by the molecular motor 130 is peeled off (unzipped) from the biomolecule-adapter molecule complex 505, and the molecular motor 130 is separated from the biomolecule-adapter molecule complex 505.

The timing at which the inside of the second liquid tank 104B is set to a stronger positive potential can be a method of automatically switching a voltage at a certain time or a method of switching a voltage using read base sequence information. Alternatively, since a decrease in blocking current can be measured when the fall-off prevention portion 113 approaches the nanopore 101, the inside of the second liquid tank 104B may be set to a stronger positive potential when the decrease in blocking current is detected. In any of these methods, the fall-off prevention portion 113 can prevent the entire biomolecule-adapter molecule complex 505 from passing through the nanopore 101 and falling off.

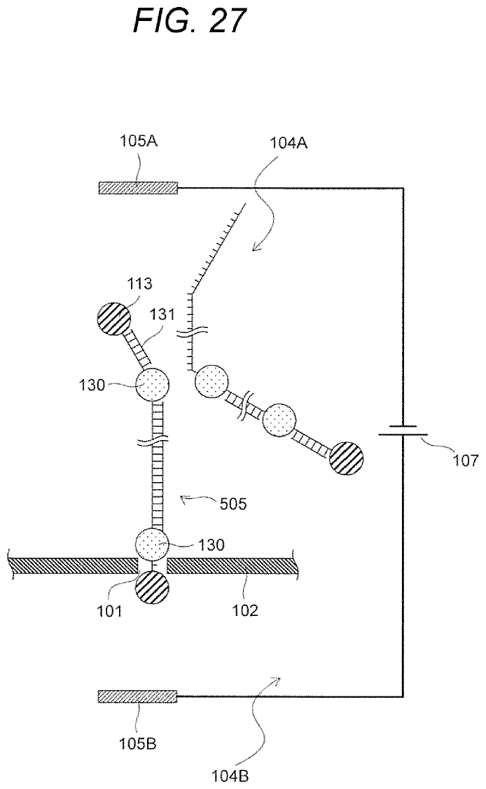

After the complementary strand 506 and the molecular motor 130 are peeled off, as illustrated in FIG. 26, the next molecular motor 130 located closest to the biomolecule 109 reaches the nanopore 101. In this state, the molecular motor 130 starts a complementary strand synthesis reaction in the direction from the 3′-terminus from the 5′-terminus starting from the 3′-terminus of the primer 131. That is, as illustrated in FIG. 27, the biomolecule-adapter molecule complex 505 is conveyed again in the direction of the first liquid tank 104A against the potential gradient by the next molecular motor 130. At this time, the base sequence information of the biomolecule-adapter molecule complex 505 passing through the nanopore 101 can be acquired again.

As described above, the base sequence information of the biomolecule 109 can be acquired a plurality of times according to the number of sets of the molecular motor 130 and the primer 131 bound to the adapter molecule 500. In a case where the adapter molecule 500 is used, the base sequence information of the biomolecule 109 can be acquired a plurality of times by the series of processing described above without performing a step of controlling to invert the voltage applied between the first liquid tank 104A and the second liquid tank 104B or binding the molecular motor 130 and the primer 131 again after one measurement. That is, in a case where the adapter molecule 500 is used, the accuracy of reading the base sequence of the biomolecule 109 can be reliably improved according to the reciprocating motion by a significantly simple operation.

### Embodiment 2-3

In the present embodiment, an adapter molecule different from the adapter molecule 400 illustrated in FIG. 16 or the like and the adapter molecule 500 illustrated in FIG. 22 or the like will be described. In the present section, the same configurations as those of the adapter molecules described in the embodiments 1-1 to 1-3 or the adapter molecule 400 or 500 are denoted by the same reference numerals, and a detailed description thereof will be omitted.

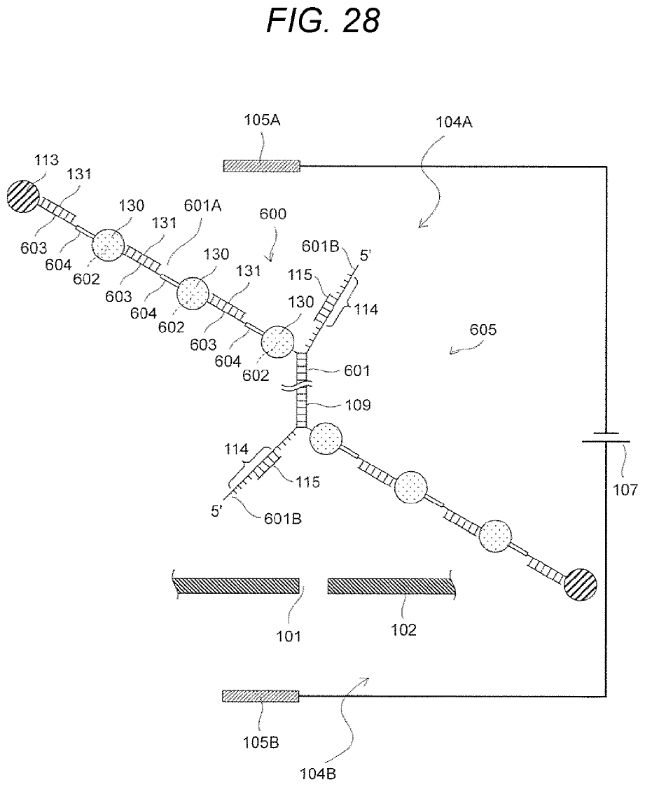

An adapter molecule 600 as illustrated in FIG. 28 comprises a double-stranded nucleic acid region 601 that binds to the biomolecule 109, and a pair of single-stranded nucleic acid regions 601A and 601B that are bound to an end different from the end binding to the biomolecule 109 in the double-stranded nucleic acid region 601 and consist of base sequences not complementary to each other. The single-stranded nucleic acid region 601A has the 3′-terminus, and the single-stranded nucleic acid region 601B has the 5′-terminus. It is preferable to provide a fall-off prevention portion 113 at the 3′-terminus of the single-stranded nucleic acid region 601A. In addition, the adapter molecule 600 illustrated in FIG. 28 comprises a three-dimensional structure formation domain 114 in the single-stranded nucleic acid region 601B. Furthermore, the adapter molecule 600 illustrated in FIG. 28 preferably comprises a three-dimensional structure formation inhibiting oligomer 115 hybridizing with the three-dimensional structure formation domain 114.

The single-stranded nucleic acid region 601A in the adapter molecule 600 illustrated in FIG. 28 comprises a plurality of molecular motor binding portions 602 to which a molecular motor 130 can bind. In addition, the single-stranded nucleic acid region 601A in the adapter molecule 600 illustrated in FIG. 28 comprises a plurality of primer binding portions 603 with which a primer 131 can hybridize on a side of the 3′-terminus of the molecular motor binding portion 602. That is, the adapter molecule 600 illustrated in FIG. 28 comprises a plurality of sets of the molecular motor binding portion 602 and the primer binding portion 503 in the single-stranded nucleic acid region 601A.

Furthermore, the single-stranded nucleic acid region 601A in the adapter molecule 600 illustrated in FIG. 28 comprises a spacer 604 between each of the plurality of sets of the molecular motor binding portion 602 and the primer binding portion 603. Here, the spacer 604 refers to a region to which the molecular motor 130 cannot be bound, that is, a region containing no base composed of AGCT. The spacer 604 is not particularly limited, and can be a linear linker containing no base. In particular, a length of the spacer 604 is preferably a length corresponding to at least 2 bases, that is, about 0.6×2 nm or more. In other words, the spacer 604 allows the molecular motor binding portion 602 and the primer binding portion 603 to be separated from each other by 2 bases or more (about 0.6×2 nm or more). Examples of a material constituting the spacer 604 can include materials that can be arranged in a DNA chain, such as C3 Spacer, PC spacer, Spacer9, Spacer18, and dSpacer provided by Integrated DNA Technologies, Inc. In addition, a linear carbon chain, a linear amino acid, a linear fatty acid, a linear sugar chain, or the like can be used as the spacer 604.

Furthermore, in the adapter molecule 600 illustrated in FIG. 28, a predetermined region in the double-stranded nucleic acid region 601 can be a labeling sequence (not illustrated). The labeling sequence is also referred to as a barcode sequence or an index sequence, and refers to a base sequence unique to the adapter molecule 600. For example, the type of the adapter molecule 600 used can be specified based on the labeling sequence by preparing a plurality of adapter molecules 600 that differ only in the labeling sequence.

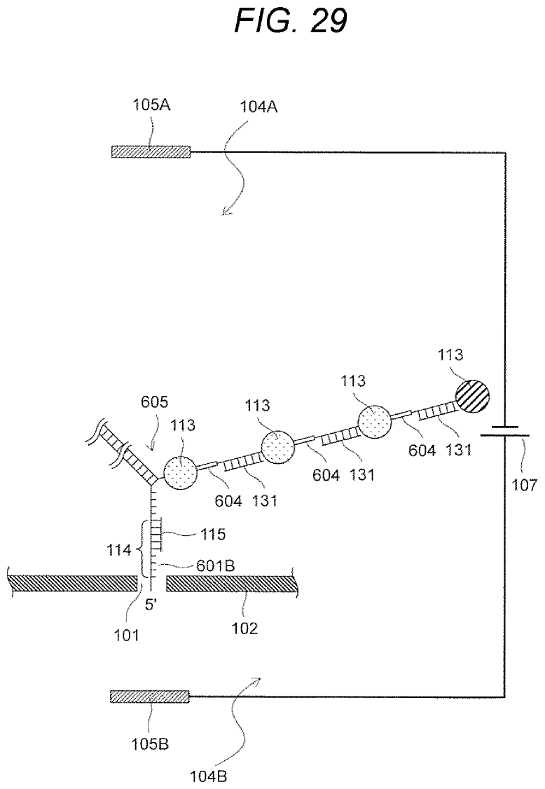

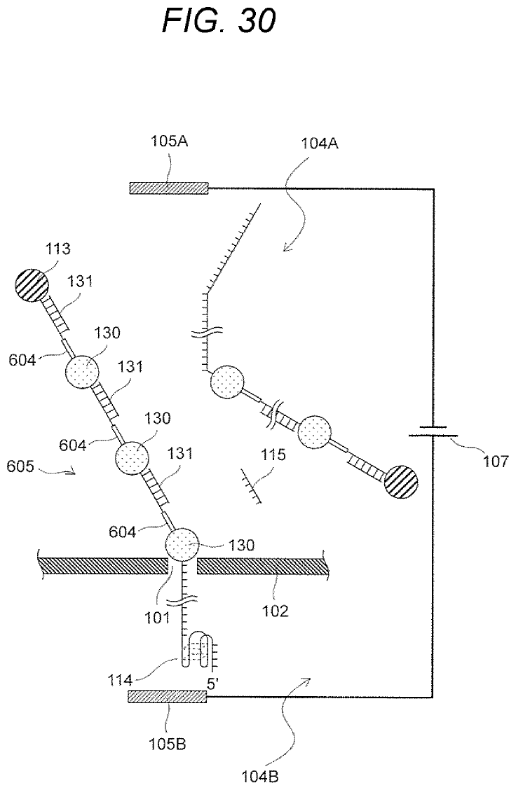

The adapter molecule 600 illustrated in FIG. 28 forms a biomolecule-adapter molecule complex 605 bound to the biomolecule 109, and a state in which the molecular motor 130 and the primer 131 are bound is illustrated. In the state illustrated in FIG. 28, a voltage is applied between the first electrode 105A and the second electrode 105B to generate a potential gradient at which the first liquid tank 104A is set to a negative potential and the second liquid tank 104B is set to a positive potential. Therefore, as illustrated in FIG. 29, the single-stranded nucleic acid region 601B is convayed in the direction of the nanopore 101, and the 5′-terminus region with which the three-dimensional structure formation inhibiting oligomer 115 does not hybridize is introduced into the nanopore 101. As illustrated in FIG. 30, the biomolecule-adapter molecule complex 605 is convayed to the second liquid tank 104B (passing) through the nanopore 101 due to the potential gradient generated between the first liquid tank 104A and the second liquid tank 104B. In this case, the double-stranded nucleic acid (i.e., the double-stranded nucleic acid region 601; the biomolecule 109; and the three-dimensional structure formation inhibiting oligomer 115 and the three-dimensional structure formation domain 114 in the adapter molecule 600) in the biomolecule-adapter molecule complex 605 is peeled off (unzipped).

As such, it is possible to obtain a single-stranded nucleic acid that can pass through the nanopore 101 without performing a complicated denaturation treatment (for example, a heat treatment) on the biomolecule 109 that is a double-stranded nucleic acid even in a case where the adapter molecule 600 is used. That is, the double-stranded nucleic acid can be easily peeled off even in the case where the adapter molecule 600 is used. In the state illustrated in FIG. 30, since the primer 131 and the molecular motor 130 are separated by the length of the spacer 604, the complementary strand synthesis reaction by the molecular motor 130 from the 3′-terminus of the primer 131 is not started. When the single-stranded nucleic acid region 601B comprising the three-dimensional structure formation domain 114 is introduced into the second liquid tank 104B, a three-dimensional structure is formed in the three-dimensional structure formation domain 114.

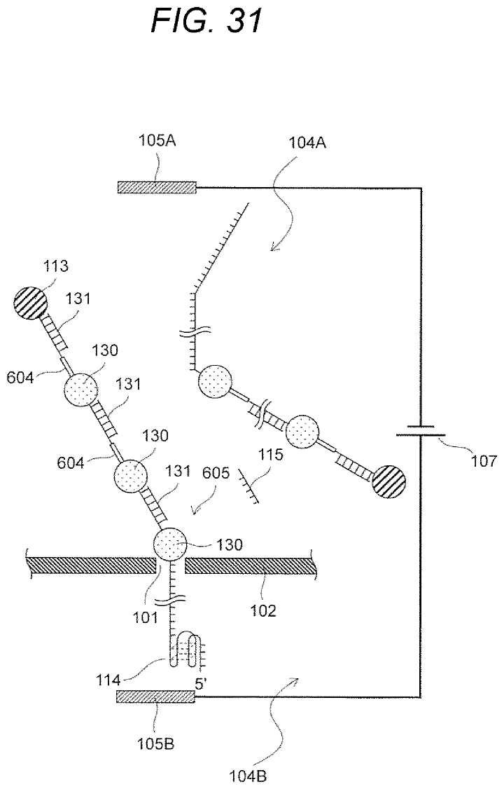

As illustrated in FIG. 30, the single-stranded biomolecule-adapter molecule complex 605 passes through the nanopore 101 due to the potential gradient generated between the first liquid tank 104A and the second liquid tank 104B, and then, the molecular motor 130 reaches the nanopore 101. Since the single-stranded biomolecule-adapter molecule complex 605 is negatively charged, the biomolecule-adapter molecule complex 605 further proceeds in a downstream direction, and a change in shape around the spacer 604 occurs. Then, the molecular motor 130 comes into contact with and binds to the 3′-terminus of the primer 131 (FIG. 31). Accordingly, the molecular motor 130 starts a complementary strand synthesis reaction in the direction from the 3′-terminus from the 5′-terminus starting from the 3′-terminus of the primer 131.

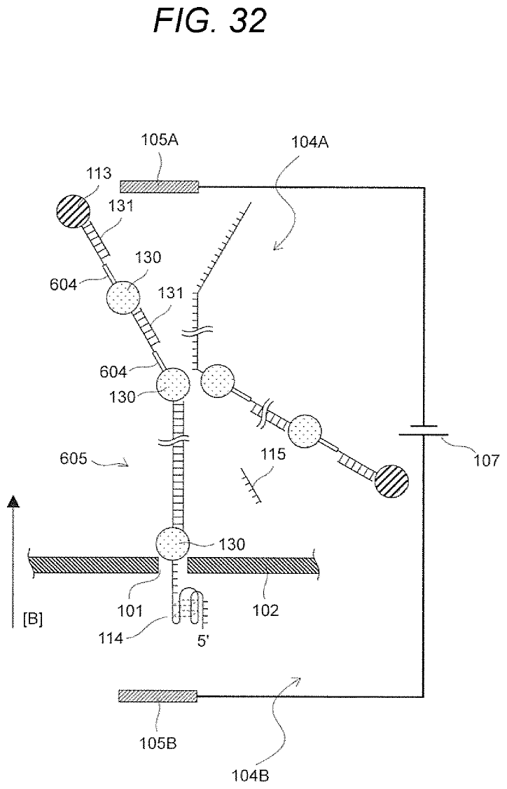

As illustrated in FIG. 32, when the complementary strand synthesis reaction by the molecular motor 130 proceeds, a force by which the single-stranded biomolecule-adapter molecule complex 605 is pulled up by the molecular motor 130 is stronger than a force by which the biomolecule-adapter molecule complex 605 is transferred to the second liquid tank 104B due to the potential gradient. Therefore, the biomolecule-adapter molecule complex 605 is conveyed in the direction of the first liquid tank 104A (the direction of the arrow B in FIG. 32) against the potential gradient. At this time, the base sequence information of the biomolecule-adapter molecule complex 605 passing through the nanopore 101 can be acquired.

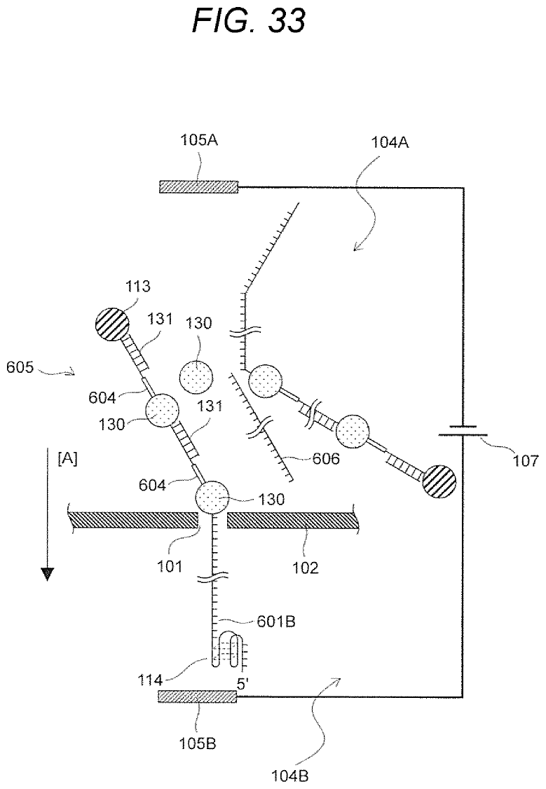

As illustrated in FIG. 32, when the three-dimensional structure formed in the single-stranded nucleic acid region 601B of the biomolecule-adapter molecule complex 605 reaches the nanopore 101, the conveyance operation by the molecular motor 130 and sequencing are stopped. The inside of the second liquid tank 104B is set to a stronger positive potential at the stage where the conveyance operation by the molecular motor 130 and sequencing are stopped. As a result, as illustrated in FIG. 33, the biomolecule-adapter molecule complex 605 is convayed to the second liquid tank 104B due to the potential gradient (a direction of the arrow A in FIG. 33). At this time, a complementary strand 606 of the biomolecule-adapter molecule complex 605 synthesized by the molecular motor 130 is peeled off (unzipped) from the biomolecule-adapter molecule complex 605, and the molecular motor 130 is separated from the biomolecule-adapter molecule complex 605.

The timing at which the inside of the second liquid tank 104B is set to a stronger positive potential can be a method of automatically switching a voltage at a certain time or a method of switching a voltage using read base sequence information. Alternatively, since a decrease in blocking current can be measured when the three-dimensional structure approaches the nanopore 101, the inside of the second liquid tank 104B may be set to a stronger positive potential when the decrease in blocking current is detected. In any of these methods, by forming a three-dimensional structure in the single-stranded nucleic acid region 601B, the entire single-stranded biomolecule-adapter molecule complex 605 can be prevented from passing through the nanopore 101.

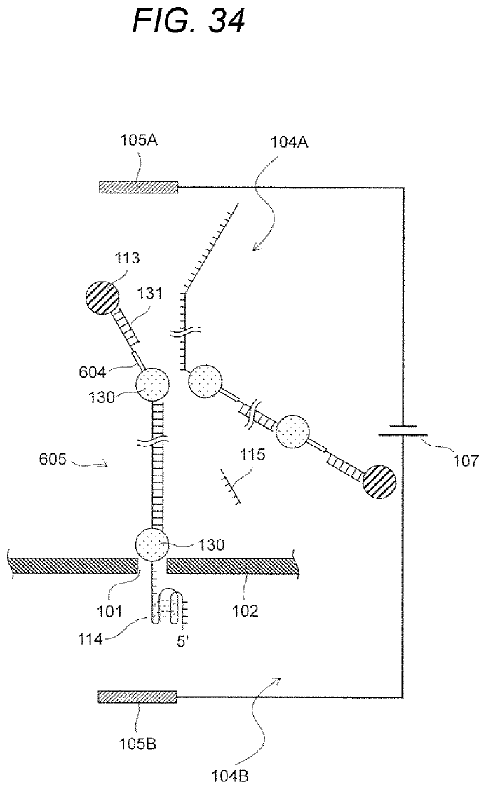

As illustrated in FIG. 33, the next molecular motor 130 located closest to the biomolecule 109 reaches the nanopore 101. The negatively charged biomolecule-adapter molecule complex 605 proceeds further downstream due to the potential gradient generated between the first liquid tank 104A and the second liquid tank 104B, and a change in shape around the spacer 604 occurs. Then, the molecular motor 130 comes into contact with and binds to the 3′-terminus of the primer 131 (see FIG. 31). Accordingly, the molecular motor 130 starts a complementary strand synthesis reaction again from the 3′-terminus of the primer 131. That is, as illustrated in FIG. 34, the biomolecule-adapter molecule complex 605 is conveyed again in the direction of the first liquid tank 104A against the potential gradient by the next molecular motor 130. At this time, the base sequence information of the biomolecule-adapter molecule complex 605 passing through the nanopore 101 can be acquired again.

As described above, the base sequence information of the biomolecule 109 can be acquired a plurality of times according to the number of sets of the molecular motor 130 and the primer 131 bound to the adapter molecule 600. In a case where the adapter molecule 600 is used, the base sequence information of the biomolecule 109 can be acquired a plurality of times by the series of processing described above without performing a step of controlling to invert the voltage applied between the first liquid tank 104A and the second liquid tank 104B or binding the molecular motor 130 and the primer 131 again after one measurement. That is, in a case where the adapter molecule 600 is used, the accuracy of reading the base sequence of the biomolecule 109 can be reliably improved according to the reciprocating motion by a significantly simple operation.

In particular, in a case where the adapter molecule 600 is used, in the second liquid tank 104B, a three-dimensional structure is formed in the vicinity of the terminus of the biomolecule-adapter molecule complex 605. Therefore, it is possible to reliably prevent the biomolecule-adapter molecule complex 605 from falling off from the nanopore 101 when the biomolecule-adapter molecule complex 605 is convayed in the direction of the direction of the first liquid tank 104A from the second liquid tank 104B. Therefore, the reading accuracy of the base sequence of the biomolecule 109 can be reliably improved according to the reciprocating motion described above.

### Embodiment 3-1

In the present embodiment, an adapter molecule comprising a molecular motor detachment induction portion of which a binding force to a molecular motor is smaller than a binding force between a biomolecule and a molecular motor, which is different from the adapter molecules illustrated in the embodiments 1-1 to 1-3 and the adapter molecules illustrated in the embodiments 2-1 to 2-3, will be described. In the adapter molecule and the like described in the present embodiment, the same configurations as those of the adapter molecules described in the embodiments 1-1 to 1-3 and the adapter molecules described in the embodiments 2-1 to 2-3 are denoted by the same reference numerals, and a detailed description thereof will be omitted in the present section.

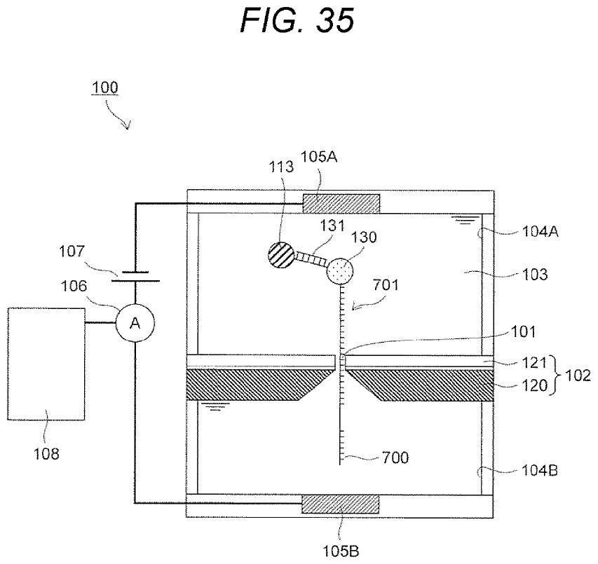

FIG. 35 illustrates a biomolecule analyzer 100 for analyzing a biomolecule-adapter molecule complex 700 comprising an adapter molecule 701 according to the present embodiment. The biomolecule analyzer 100 is a device that analyzes the biomolecule-adapter molecule complex 701, and is a device for biomolecule analysis that measures an ion current by a blocking current method. The biomolecule analyzer 100 comprises a substrate 102 in which a nanopore 101 is formed, a pair of liquid tanks 104 (a first liquid tank 104A and a second liquid tank 104B) arranged to be in contact with the substrate 102 with the substrate 102 interposed therebetween and filled with an electrolyte solution 103 therein, and a pair of electrodes 105 (a first electrode 105A and a second electrode 105B) in contact with the first liquid tank 104A and the second liquid tank 104B, respectively. At the time of measurement, a predetermined voltage is applied from a voltage source 107 between the pair of electrodes 105, and a current flows between the pair of electrodes 105. A magnitude of the current flowing between the electrodes 105 is measured by an ammeter 106, and a measured value thereof is analyzed by a computer 108.

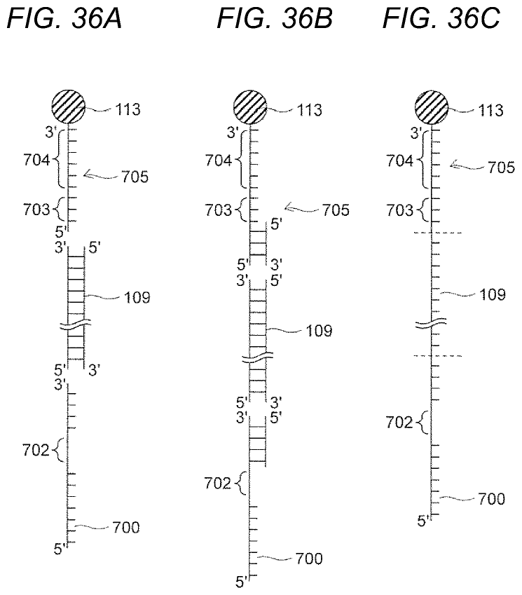

As illustrated in FIGS. 36(A) and 36(B), the adapter molecule 700 comprises a molecular motor detachment induction portion 702 in the molecule. The molecular motor detachment induction portion 702 is a region having a feature in which a binding force to a molecular motor 130 is smaller than a binding force between a biomolecule 109 and the molecular motor 130. The molecular motor detachment induction portion 702 is not particularly limited, and can be a region consisting of a carbon chain that does not contain a phosphodiester bond or abasic sequence. Here, the molecular motor 130 such as DNA polymerase binds to a nucleic acid in which nucleotides are bound by phosphodiester bonds. Therefore, the molecular motor detachment induction portion 702 can have a structure different from that of the nucleic acid, that is, as an example, a chain structure excluding a structure in which monomers are bound by the phosphodiester bonds. The molecular motor detachment induction portion 702 more preferably has a structure having no base. As an example, the molecular motor detachment induction portion 702 can be composed of an iSpC3-based abasic site. In this case, since a phosphate group is disposed in a size equal to or smaller than a size of a molecular motor bond (for example, polymerase), it is preferable to have a phosphate group-absent region with a length equal to or longer than a physical dimension of an average molecular motor. As an example, iSp9 or iSp18 can be used. In addition, the molecular motor detachment induction portion 702 may be one in which a plurality of types thereof are regularly or randomly linked to each other. Furthermore, the molecular motor detachment induction portion 702 is not limited to one composed of an abasic site as described above, and may be a carbon chain having any length or polyethylene glycol (PEG) having any length. In addition, the molecular motor detachment induction portion 702 may be a modified base having a phosphate group as long as it can suppress and detach an extension reaction by the polymerase. As such an example, Nitroindole can be exemplified. The extension reaction of the polymerase can be stopped using Nitroindole for the molecular motor detachment induction portion 702.

The adapter molecule 700 may consist of single-stranded DNA as illustrated in FIG. 36(A), or in a case where the biomolecule 109 to be analyzed is double-stranded DNA as illustrated in FIG. 36(B), the end bound to the biomolecule 109 may be double-stranded DNA.

The adapter molecule 700 is bound to one end of the biomolecule 109 to be analyzed. An adapter molecule 705 (hereinafter, referred to as an “adapter molecule 705 for molecular motor binding”) comprising a molecular motor binding portion 703 to which the molecular motor 130 binds and a primer binding portion 704 with which the primer 131 can hybridize is bound to the other end of the biomolecule 109. The adapter molecule 705 for molecular motor binding preferably comprises a fall-off prevention portion 113 at an end (for example, the 3′-terminus) opposite to an end bound to the biomolecule 109.

In the examples illustrated in FIGS. 36(A) and 36(B), the adapter molecule 700 is bound to the 5′-terminus of the biomolecule 109, and the adapter molecule 705 for molecular motor binding is bound to the 3′-terminus of the biomolecule 109. Any one of the adapter molecule 700 illustrated in FIGS. 36(A) and 36(B) and the adapter molecule 705 for molecular motor binding may be used, and the single-stranded biomolecule-adapter molecule complex 701 can be prepared as illustrated in FIG. 36(C) by forming a double-stranded region into a single strand.

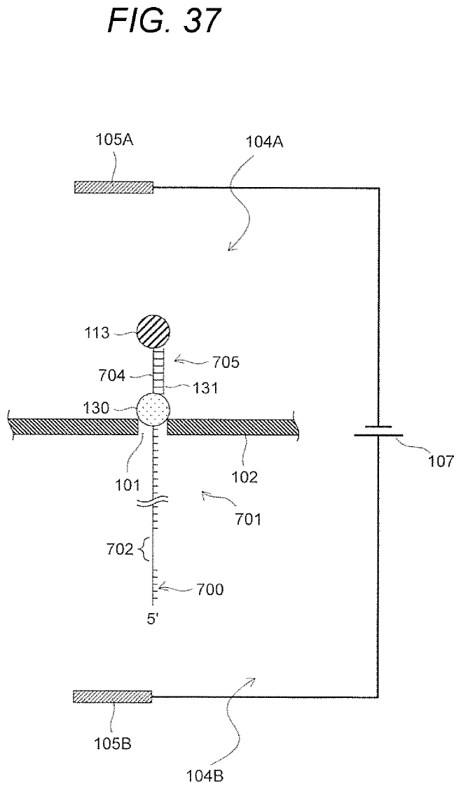

A method for analyzing the biomolecule 109 using the adapter molecule 700 configured as described above will be described with reference to FIGS. 37 to 39.

First, a biomolecule-adapter molecule complex 701 in which the adapter molecule 700 is bonded to one end of the biomolecule 109 and the adapter molecule 705 for molecular motor binding is bonded to the other end of the biomolecule 109 is prepared. The first liquid tank 104A is filled with an electrolyte solution containing the biomolecule-adapter molecule complex 701, the molecular motor 130, and the primer 131. Accordingly, the molecular motor 130 is bound to the molecular motor binding portion 703 in the adapter molecule 705 for molecular motor binding, and the primer 131 hybridizes with the primer binding portion 704.

Next, a voltage is applied between the first electrode 105A and the second electrode 105B to generate a potential gradient at which the first liquid tank 104A is set to a negative potential and the second liquid tank 104B is set to a positive potential. Therefore, the end of the adapter molecule 700 in the biomolecule-adapter molecule complex 701 is convayed in the direction of the nanopore 101 to be introduced into the nanopore 101. The biomolecule-adapter molecule complex 701 is convayed to the second liquid tank 104B (passing) through the nanopore 101 due to the potential gradient generated between the first liquid tank 104A and the second liquid tank 104B. Although not illustrated, the fall-off prevention portion 113 is added to the electrolyte solution 103 in the second liquid tank 104B, such that the fall-off prevention portion 113 can be added to the end of the biomolecule-adapter molecule complex 401 that has convayed to the second liquid tank 104B.

As illustrated in FIG. 37, the biomolecule-adapter molecule complex 701 passes through the nanopore 101 due to the potential gradient generated between the first liquid tank 104A and the second liquid tank 104B, and then, the molecular motor 130 bound to the molecular motor binding portion 703 reaches the nanopore 101. In this state, the molecular motor 130 starts a complementary strand synthesis reaction in the direction from the 3′-terminus from the 5′-terminus starting from the 3′-terminus of the primer 131.

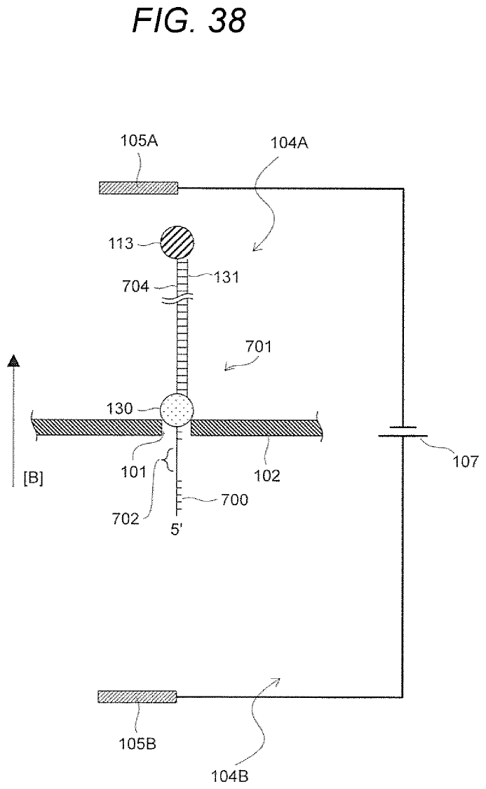

As illustrated in FIG. 38, when the complementary strand synthesis reaction by the molecular motor 130 proceeds, a force by which the biomolecule-adapter molecule complex 701 is pulled up by the molecular motor 130 is stronger than a force by which the biomolecule-adapter molecule complex 701 is convayed to the second liquid tank 104B due to the potential gradient. Therefore, the biomolecule-adapter molecule complex 701 is conveyed in the direction of the first liquid tank 104A (the direction of the arrow B in FIG. 38) against the potential gradient. At this time, the base sequence information of the biomolecule-adapter molecule complex 701 passing through the nanopore 101 can be acquired.

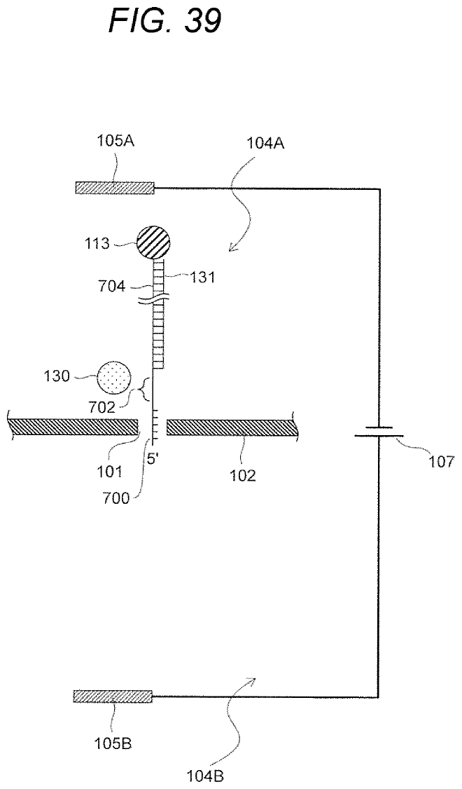

When the biomolecule-adapter molecule complex 701 is continuously conveyed in the direction of the first liquid tank 104A by the molecular motor 130, and the molecular motor 130 comes to the molecular motor detachment induction portion 702 as illustrated in FIG. 39, the molecular motor 130 is separated from the biomolecule-adapter molecule complex 701. When the molecular motor 130 is separated from the biomolecule-adapter molecule complex 701, the biomolecule-adapter molecule complex 701 having a complementary strand 706 is convayed in the direction of the second liquid tank 104B due to the potential gradient generated between the first liquid tank 104A and the second liquid tank 104B, and the complementary strand 706 is peeled off (unzipped) from the biomolecule-adapter molecule complex 701.

As described above, since the molecular motor 130 is easily separated from the biomolecule-adapter molecule complex 701 by using the adapter molecule 700, it is not necessary to forcibly separate the molecular motor 130 and peel off the synthesized complementary strand by setting the inside of the second liquid tank 104B to a stronger positive potential. Furthermore, by using the adapter molecule 700, the molecular motor 130 is easily separated from the biomolecule-adapter molecule complex 701, and then, the biomolecule-adapter molecule complex 701 is convayed in the direction of the second liquid tank 104B. Therefore, it is possible to prevent the biomolecule-adapter molecule complex 701 from falling off even without comprising the fall-off prevention portion 113 at the end of the adapter molecule 700.

In addition, although not illustrated, after the synthesized complementary strand 706 is peeled off, the first liquid tank 104A and the second liquid tank 104B are set to have opposite potential gradients (the first liquid tank 104A is set to a positive potential and the second liquid tank 104B is set to a negative potential), such that the biomolecule-adapter molecule complex 701 can be convayed in the direction of the first liquid tank 104A, and the molecular motor 130 and the primer 131 can be bound to a predetermined position of the adapter molecule 705 for molecular motor binding again. Thereafter, the base sequence information of the biomolecule 109 can be obtained again according to the steps illustrated in FIGS. 37 to 39.

In a case where the adapter molecule 700 is used as described above, the processing of separating the molecular motor 130 by controlling the voltage gradient generated between the first liquid tank 104A and the second liquid tank 104B and peeling off the complementary strand 706 is unnecessary, and the reading accuracy for the base sequence of the biomolecule 109 can be reliably improved according to the reciprocating motion by a significantly simple operation.

### Embodiment 3-2

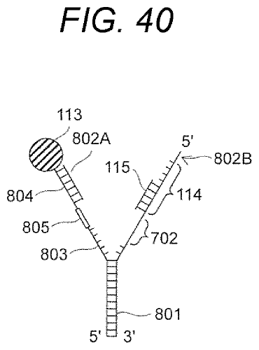

In the present embodiment, an adapter molecule 800 as illustrated in FIG. 40, which is different from each of the adapter molecule 700 and the adapter molecule 705 for molecular motor binding illustrated in FIGS. 36(A) and 36(B), will be described. In the adapter molecule 800 exemplarily illustrated in FIG. 40 and the biomolecule analyzer using the same, the same components as those of the adapter molecule 700 and the adapter molecule 705 for molecular motor binding illustrated in FIGS. 36(A) and 36(B) are denoted by the same reference numerals, and a detailed description thereof will be omitted in this section.

The adapter molecule 800 illustrated in FIG. 40 comprises a double-stranded nucleic acid region 801 that directly binds to the biomolecule 109, and a pair of single-stranded nucleic acid regions 802A and 802B that are bound to an end different from the end bound to the biomolecule 109 in the double-stranded nucleic acid region 801 and consist of base sequences not complementary to each other. The single-stranded nucleic acid region 802A has a fall-off prevention portion 113 bound to the 3′-terminus, and the single-stranded nucleic acid region 802B has the 5′-terminus. In addition, the adapter molecule 800 illustrated in FIG. 40 comprises a three-dimensional structure formation domain 114 in the single-stranded nucleic acid region 802B. Furthermore, the adapter molecule 800 illustrated in FIG. 40 preferably comprises a three-dimensional structure formation inhibiting oligomer 115 hybridizing with the three-dimensional structure formation domain 114. Furthermore, the adapter molecule 800 comprises a molecular motor detachment induction portion 702 at a position closer to the double-stranded nucleic acid region 801 than the three-dimensional structure formation domain 114 in the single-stranded nucleic acid region 801B.

The single-stranded nucleic acid region 801A in the adapter molecule 800 illustrated in FIG. 40 comprises a molecular motor binding portion 803 to which a molecular motor can bind. In addition, the single-stranded nucleic acid region 801A in the adapter molecule 800 illustrated in FIG. 40 has a primer binding portion 803 with which a primer can hybridize on a side of the 3′-terminus of the molecular motor binding portion 804. The primer binding portion 804 is not limited to a specific base sequence as long as it has a sequence complementary to the base sequence of the primer to be used. Here, the primer is not particularly limited, and an example thereof can include a single-stranded nucleotide having a base length of 10 to 40, preferably 15 to 35, and more preferably 18 to 25. Therefore, the primer binding portion 303 can be a region composed of a base sequence complementary to a base sequence having a base length of 10 to 40, preferably 15 to 35, and more preferably 18 to 25.

Furthermore, the single-stranded nucleic acid region 802A in the adapter molecule 800 illustrated in FIG. 40 comprises a spacer 805 between the molecular motor binding portion 803 and the primer binding portion 804. Here, the spacer 805 refers to a region to which the molecular motor cannot be bound, that is, a region containing no base composed of AGCT. The spacer 805 is not particularly limited, and can be a linear linker containing no base. In particular, a length of the spacer 805 is preferably a length corresponding to at least 2 bases, that is, about 0.6×2 nm or more. In other words, the spacer 805 allows the molecular motor binding portion 803 and the primer binding portion 804 to be separated from each other by 2 bases or more (about 0.6×2 nm or more). Examples of a material constituting the spacer 805 can include materials that can be arranged in a DNA chain, such as C3 Spacer, PC spacer, Spacer9, Spacer18, and dSpacer provided by Integrated DNA Technologies, Inc. In addition, a linear carbon chain, a linear amino acid, a linear fatty acid, a linear sugar chain, or the like can be used as the spacer 805.

Furthermore, in the adapter molecule 800 illustrated in FIG. 40, a predetermined region in the double-stranded nucleic acid region 801 can be a labeling sequence (not illustrated). The labeling sequence is also referred to as a barcode sequence or an index sequence, and refers to a base sequence unique to the adapter molecule 800. For example, the type of the adapter molecule 800 used can be specified based on the labeling sequence by preparing a plurality of adapter molecules 800 that differ only in the labeling sequence.

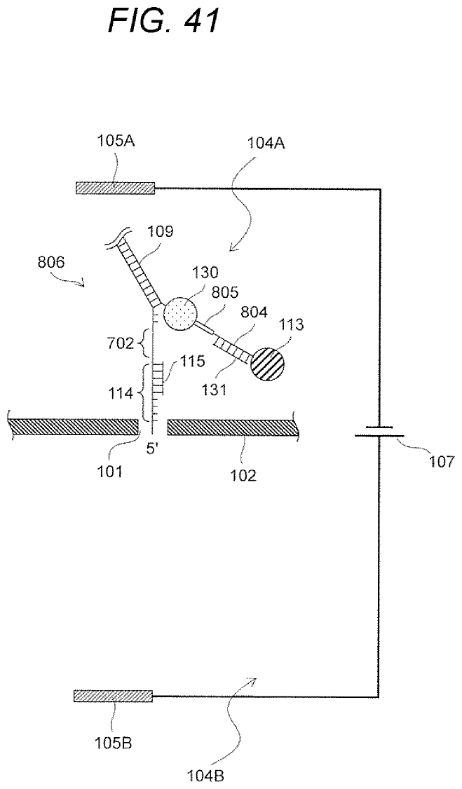

A method for analyzing the biomolecule 109 using the adapter molecule 800 configured as described above will be described with reference to FIGS. 41 to 45.

First, a biomolecule-adapter molecule complex 806 in which the adapter molecule 800 is bonded to each of both ends of the biomolecule 109 is prepared. The first liquid tank 104A is filled with an electrolyte solution containing the biomolecule-adapter molecule complex 806, the molecular motor 130, the primer 131, and the three-dimensional structure formation inhibiting oligomer 115. Accordingly, as illustrated in FIG. 41, the molecular motor 130 binds to the molecular motor binding portion 803 in the adapter molecule 800, the primer 131 hybridizes with the primer binding portion 804, and the three-dimensional structure formation inhibiting oligomer 115 hybridizes with the three-dimensional structure formation domain 114 of the single-stranded nucleic acid region 802B.

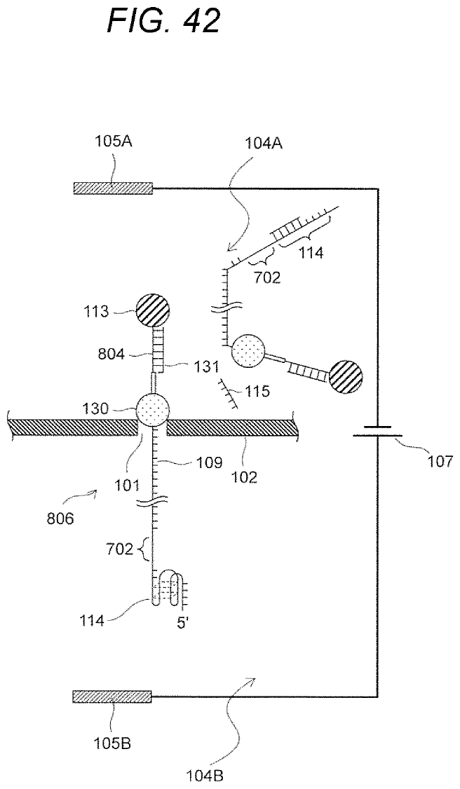

Next, a voltage is applied between the first electrode 105A and the second electrode 105B to generate a potential gradient at which the first liquid tank 104A is set to a negative potential and the second liquid tank 104B is set to a positive potential. Therefore, the tip end of the single-stranded nucleic acid region 802B is convayed in the direction of the nanopore 101, and the 5′-terminus region with which the three-dimensional structure formation inhibiting oligomer 115 does not hybridize is introduced into the nanopore 101. As illustrated in FIG. 42, the biomolecule-adapter molecule complex 806 is convayed to the second liquid tank 104B (passing) through the nanopore 101 due to the potential gradient generated between the first liquid tank 104A and the second liquid tank 104B. In this case, the double-stranded nucleic acid (i.e., the double-stranded nucleic acid region 801; the biomolecule 109; and the three-dimensional structure formation inhibiting oligomer 115 and the three-dimensional structure formation domain 114 in the adapter molecule 800) in the biomolecule-adapter molecule complex 806 is peeled off (unzipped).

As such, it is possible to obtain a single-stranded nucleic acid that can pass through the nanopore 101 without performing a complicated denaturation treatment (for example, a heat treatment) on the biomolecule 109 that is a double-stranded nucleic acid even in a case where the adapter molecule 800 is used. That is, the double-stranded nucleic acid can be easily peeled off even in the case where the adapter molecule 800 is used. In the states illustrated in FIGS. 41 and 42, since the primer 131 and the molecular motor 130 are separated by the length of the spacer 805, the complementary strand synthesis reaction by the molecular motor 130 starting from the 3′-terminus of the primer 131 is not started. When the single-stranded nucleic acid region 802B comprising the three-dimensional structure formation domain 114 is introduced into the second liquid tank 104B, a three-dimensional structure is formed in the three-dimensional structure formation domain 114.

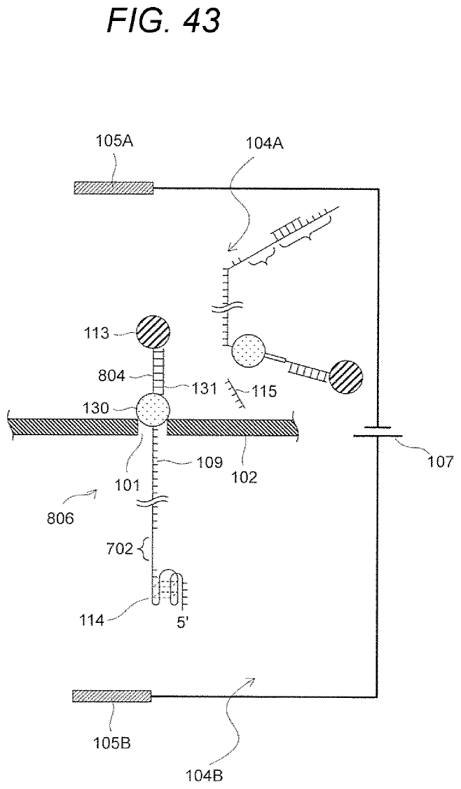

As illustrated in FIG. 42, the single-stranded biomolecule-adapter molecule complex 806 passes through the nanopore 101 due to the potential gradient generated between the first liquid tank 104A and the second liquid tank 104B, and then, the molecular motor 130 reaches the nanopore 101. Since the single-stranded biomolecule-adapter molecule complex 806 is negatively charged, the biomolecule-adapter molecule complex 806 further proceeds in a downstream direction, and a change in shape around the spacer 805 occurs. Then, the molecular motor 130 comes into contact with and binds to the 3′-terminus of the primer 131 (FIG. 43). Accordingly, the molecular motor 130 starts a complementary strand synthesis reaction in the direction from the 3′-terminus from the 5′-terminus starting from the 3′-terminus of the primer 131.

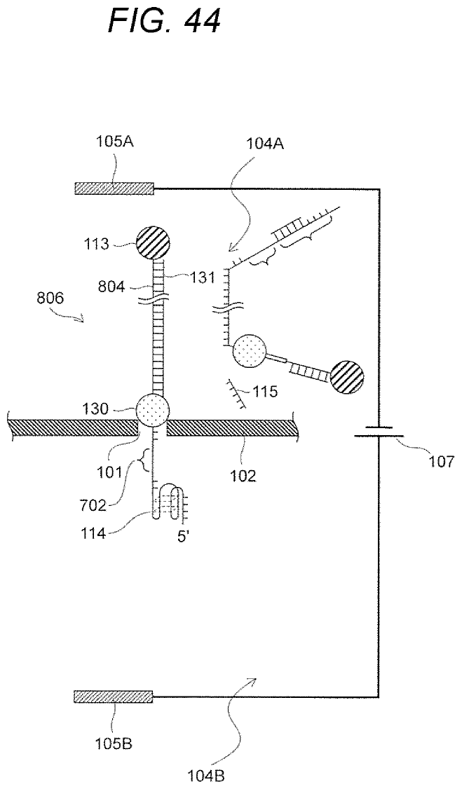

As illustrated in FIG. 44, when the complementary strand synthesis reaction by the molecular motor 130 proceeds, a force by which the single-stranded biomolecule-adapter molecule complex 805 is pulled up by the molecular motor 130 is stronger than a force by which the biomolecule-adapter molecule complex 805 is convayed to the second liquid tank 104B due to the potential gradient. Therefore, the single-stranded biomolecule-adapter molecule complex 805 is conveyed in the direction of the first liquid tank 104A against the potential gradient. At this time, the base sequence information of the biomolecule-adapter molecule complex 806 passing through the nanopore 101 can be acquired.

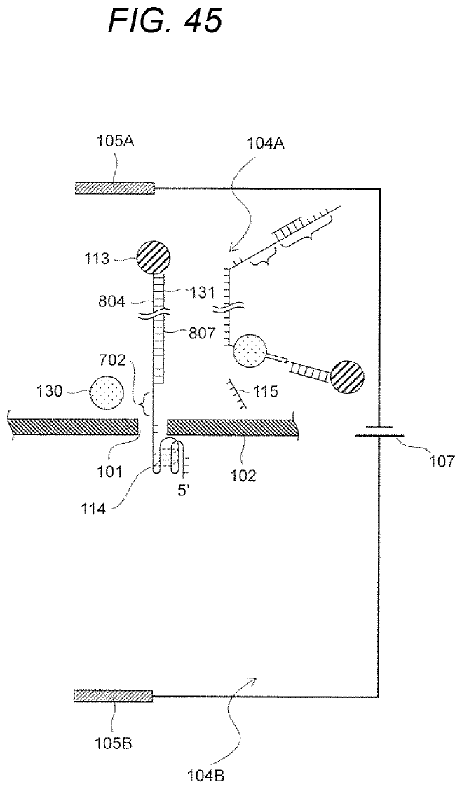

When the biomolecule-adapter molecule complex 806 is continuously conveyed in the direction of the first liquid tank 104A by the molecular motor 130, and the molecular motor 130 comes to the molecular motor detachment induction portion 702 while the three-dimensional structure formed in the single-stranded nucleic acid region 802B reaches the nanopore 101 as illustrated in FIG. 45, the molecular motor 130 is separated from the biomolecule-adapter molecule complex 806. Although not illustrated, when the molecular motor 130 is separated from the biomolecule-adapter molecule complex 806, the biomolecule-adapter molecule complex 806 having a complementary strand 807 is convayed in the direction of the second liquid tank 104B due to the potential gradient generated between the first liquid tank 104A and the second liquid tank 104B, and the complementary strand 807 is peeled off (unzipped) from the biomolecule-adapter molecule complex 805.

In a case where the adapter molecule 800 is used, since the molecular motor 130 is easily separated from the biomolecule-adapter molecule complex 806, it is not necessary to forcibly separate the molecular motor 130 and peel off the synthesized complementary strand 807 by setting the inside of the second liquid tank 104B to a stronger positive potential. Furthermore, in the case where the adapter molecule 800 is used, a three-dimensional structure is formed in the vicinity of the end of the biomolecule-adapter molecule complex 806 in the second liquid tank 104B. Therefore, it is possible to reliably prevent the biomolecule-adapter molecule complex 806 from falling off from the nanopore 101.

In addition, although not illustrated, after the synthesized complementary strand 807 is peeled off, a voltage applied between the first electrode 105A and the second electrode 105B is inverted to generate a potential gradient at which the first liquid tank 104A is set to a positive potential and the second liquid tank 104B is set to a negative potential. Accordingly, the single-stranded biomolecule-adapter molecule complex 806 can be convayed from the second liquid tank 104B toward the first liquid tank 104A through the nanopore 101. Thereafter, the molecular motor 130 and the primer 131 are added to the electrolyte solution 103 filled in the first liquid tank 104A, the primer 131 is allowed to hybridize with the primer binding portion 804, and the molecular motor 130 is bound to the molecular motor binding portion 803. Thereafter, the voltage applied between the first electrode 105A and the second electrode 105B is inverted again to generate a potential gradient at which the first liquid tank 104A is set to a negative potential and the second liquid tank 104B is set to a positive potential. The biomolecule-adapter molecule complex 806 with which the primer 131 hybridizes, and to which the molecular motor 130 is bound is convayed in the direction of the second liquid tank 104B. As illustrated in FIG. 43, a change in shape around the spacer 805 occurs, and thus, a state in which the molecular motor 130 is in contact with the 3′-terminus of the primer 131 is formed. That is, sequencing can be performed for each conveyance operation by the molecular motor 130 by repeating FIGS. 41 to 45.

In a case where the adapter molecule 800 is used as described above, the processing of separating the molecular motor 130 by controlling the voltage gradient generated between the first liquid tank 104A and the second liquid tank 104B and peeling off the complementary strand 807 is unnecessary, and the reading accuracy for the base sequence of the biomolecule 109 can be reliably improved according to the reciprocating motion by a significantly simple operation.

### Embodiment 3-3

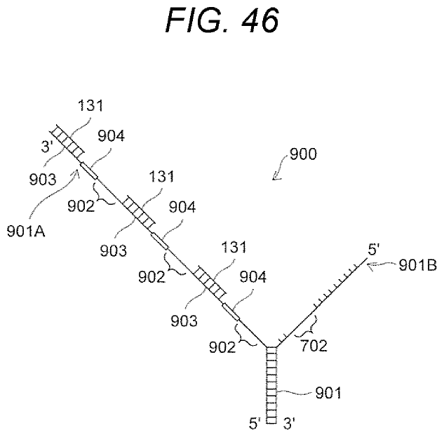

In the present embodiment, an adapter molecule 900 as illustrated in FIG. 46, which is different from each of the adapter molecule 700 illustrated in FIGS. 36(A) and 36(B) and the adapter molecule 800 illustrated in FIG. 40, will be described. In the adapter molecule 900 exemplarily illustrated in FIG. 46 and the biomolecule analyzer using the same, the same components as those of the adapter molecule 700 illustrated in FIGS. 36(A) and 36(B) and the adapter molecule 800 illustrated in FIG. 40 are denoted by the same reference numerals, and a detailed description thereof will be omitted in this section.

The adapter molecule 900 illustrated in FIG. 46 comprises a double-stranded nucleic acid region 901 that binds to the biomolecule 109, and a pair of single-stranded nucleic acid regions 901A and 901B that are bound to an end different from the end binding to the biomolecule 109 in the double-stranded nucleic acid region 901 and consist of base sequences not complementary to each other. The single-stranded nucleic acid region 901A has the 3′-terminus, and the single-stranded nucleic acid region 901B has the 5′-terminus. Although not illustrated, it is also possible to provide a fall-off prevention portion (a fall-off prevention portion 113 in FIG. 40 and the like) at the terminus of the single-stranded nucleic acid region 901A. In the adapter molecule 900 illustrated in FIG. 46, the single-stranded nucleic acid region 901B comprises a molecular motor detachment induction portion 702.

The single-stranded nucleic acid region 901A in the adapter molecule 900 illustrated in FIG. 46 has a plurality of molecular motor binding portions 902 to which a molecular motor 130 can bind. In addition, the single-stranded nucleic acid region 901A in the adapter molecule 900 illustrated in FIG. 46 has a plurality of primer binding portions 903 with which a primer 131 can hybridize on a side of the 3′-terminus of the molecular motor binding portion 902. That is, the adapter molecule 900 illustrated in FIG. 46 comprises a plurality of sets of the molecular motor binding portion 902 and the primer binding portion 903 in the single-stranded nucleic acid region 901A.

Furthermore, the single-stranded nucleic acid region 901A in the adapter molecule 900 illustrated in FIG. 46 comprises a spacer 904 between each of the plurality of sets of the molecular motor binding portion 902 and the primer binding portion 903. Here, the spacer 904 refers to a region to which the molecular motor 130 cannot be bound, that is, a region containing no base composed of AGCT. The spacer 904 is not particularly limited, and can be a linear linker containing no base. In particular, a length of the spacer 904 is preferably a length corresponding to at least two bases, that is, about 0.6×2 nm or more. In other words, the spacer 904 allows the molecular motor binding portion 902 and the primer binding portion 903 to be separated from each other by 2 bases or more (about 0.6×2 nm or more). Examples of a material constituting the spacer 904 can include materials that can be arranged in a DNA chain, such as C3 Spacer, PC spacer, Spacer9, Spacer18, and dSpacer provided by Integrated DNA Technologies, Inc. In addition, a linear carbon chain, a linear amino acid, a linear fatty acid, a linear sugar chain, or the like can be used as the spacer 904.

Furthermore, in the adapter molecule 900 illustrated in FIG. 46, a predetermined region in the double-stranded nucleic acid region 901 can be a labeling sequence (not illustrated). The labeling sequence is also referred to as a barcode sequence or an index sequence, and refers to a base sequence unique to the adapter molecule 900. For example, the type of the adapter molecule 900 used can be specified based on the labeling sequence by preparing a plurality of adapter molecules 900 that differ only in the labeling sequence.

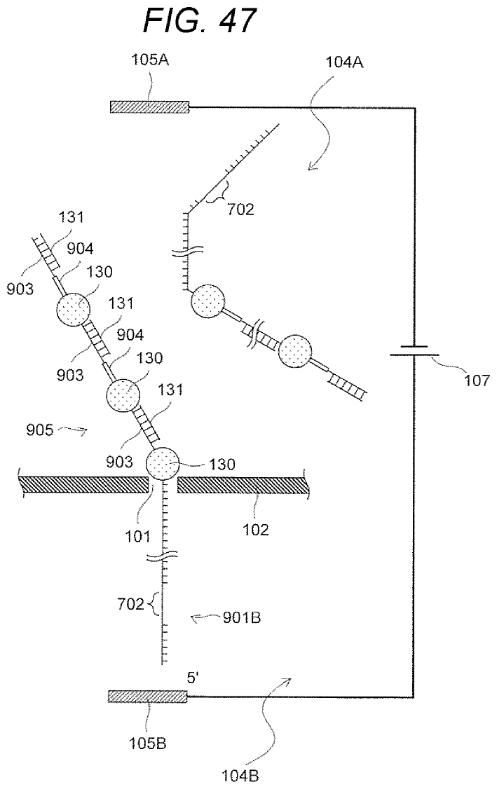

A method for analyzing the biomolecule 109 using the adapter molecule 900 configured as described above will be described with reference to FIGS. 47 to 49.

First, a biomolecule-adapter molecule complex 905 in which the adapter molecule 900 illustrated in FIG. 46 is bonded to each of both ends of the biomolecule 109 is prepared. The biomolecule-adapter molecule complex 905 is filled in the first liquid tank 10A together with the molecule probe 130 and the primer 131. In this state, a voltage is applied between the first electrode 105A and the second electrode 105B to generate a potential gradient at which the first liquid tank 104A is set to a negative potential and the second liquid tank 104B is set to a positive potential. Accordingly, as illustrated in FIG. 47, the single-stranded nucleic acid region 901B is convayed in the direction of the nanopore 101, and the double-stranded nucleic acid (i.e., the double-stranded nucleic acid region 901 and the biomolecule 109 in the adapter molecule 900) is peeled off (unzipped). In addition, as illustrated in FIG. 47, the molecular motor 130 located closest to the biomolecule 109 in the biomolecule-adapter molecule complex 905 reaches the nanopore 101. In this state, the molecular motor 130 starts a complementary strand synthesis reaction in the direction from the 3′-terminus from the 5′-terminus starting from the 3′-terminus of the primer 131.

As such, it is possible to obtain a single-stranded nucleic acid that can pass through the nanopore 101 without performing a complicated denaturation treatment (for example, a heat treatment) on the biomolecule 109 that is a double-stranded nucleic acid even in a case where the adapter molecule 900 is used. That is, the double-stranded nucleic acid can be easily peeled off even in the case where the adapter molecule 900 is used.

When the complementary strand synthesis reaction by the molecular motor 130 proceeds, the single-stranded biomolecule-adapter molecule complex 905 is conveyed in the direction of the first liquid tank 104A against the potential gradient. At this time, the base sequence information of the biomolecule-adapter molecule complex 905 passing through the nanopore 101 can be acquired.

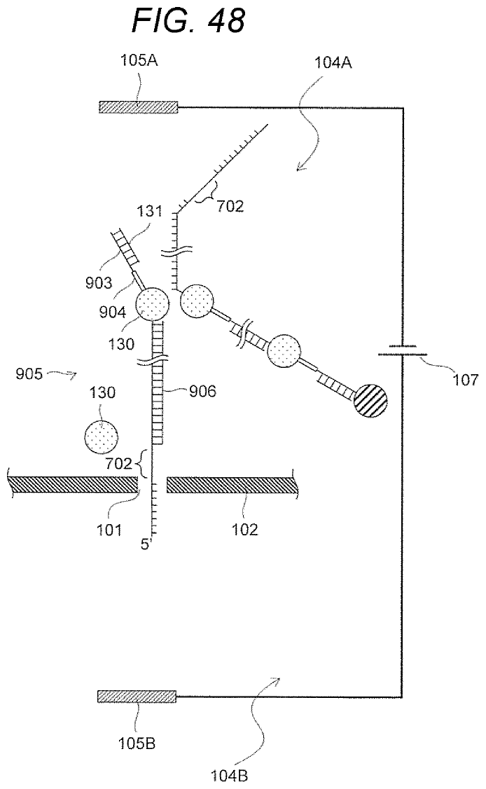

When the biomolecule-adapter molecule complex 905 is continuously conveyed in the direction of the first liquid tank 104A by the molecular motor 130, and the molecular motor 130 comes to the molecular motor detachment induction portion 702 formed in the single-stranded nucleic acid region 901B as illustrated in FIG. 48, the molecular motor 130 is separated from the biomolecule-adapter molecule complex 905. Although not illustrated, when the molecular motor 130 is separated from the biomolecule-adapter molecule complex 905, the biomolecule-adapter molecule complex 905 having a complementary strand 906 is transferred in the direction of the second liquid tank 104B due to the potential gradient generated between the first liquid tank 104A and the second liquid tank 104B, and the complementary strand 906 is peeled off (unzipped) from the biomolecule-adapter molecule complex 905.

Even in a case where the adapter molecule 900 is used, since the molecular motor 130 is easily separated from the biomolecule-adapter molecule complex 905 by the molecular motor detachment induction portion 702, it is not necessary to forcibly separate the molecular motor 130 and peel off the synthesized complementary strand 906 by setting the inside of the second liquid tank 104B to a stronger positive potential.

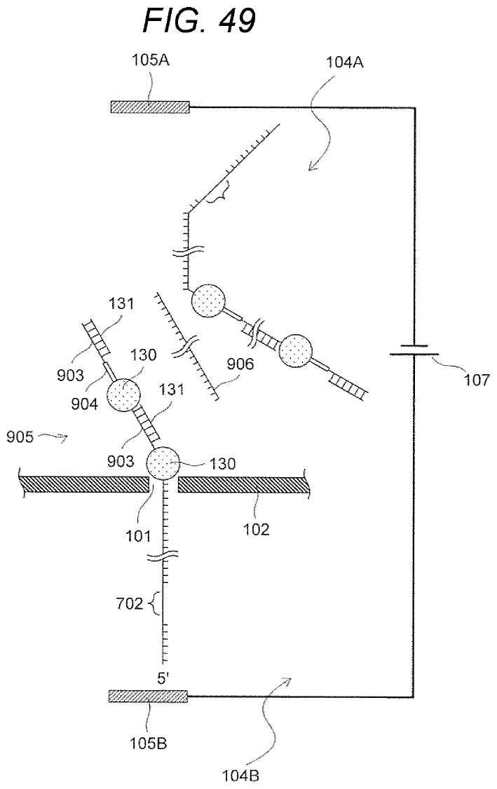

The next molecular motor 130 located closest to the biomolecule 109 reaches the nanopore 101. The negatively charged biomolecule-adapter molecule complex 905 proceeds further downstream due to the potential gradient generated between the first liquid tank 104A and the second liquid tank 104B, and as illustrated in FIG. 49, a change in shape around the spacer 904 occurs, and the molecular motor 130 comes into contact with and binds to the 3′-terminus of the primer 131. Accordingly, the molecular motor 130 starts a complementary strand synthesis reaction again from the 3′-terminus of the primer 131. That is, the biomolecule-adapter molecule complex 905 is conveyed again in the direction of the first liquid tank 104A against the potential gradient by the next molecular motor 130. At this time, the base sequence information of the biomolecule-adapter molecule complex 905 passing through the nanopore 101 can be acquired again.

As described above, the base sequence information of the biomolecule 109 can be acquired a plurality of times according to the number of sets of the molecular motor 130 and the primer 131 bound to the adapter molecule 900. In a case where the adapter molecule 900 is used, the base sequence information of the biomolecule 109 can be acquired a plurality of times by the series of processing described above without performing a step of controlling to invert the voltage applied between the first liquid tank 104A and the second liquid tank 104B or binding the molecular motor 130 and the primer 131 again after one measurement. That is, in a case where the adapter molecule 900 is used, the accuracy of reading the base sequence of the biomolecule 109 can be reliably improved according to the reciprocating motion by a significantly simple operation.

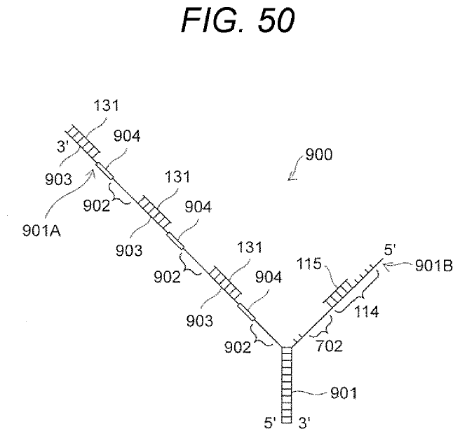

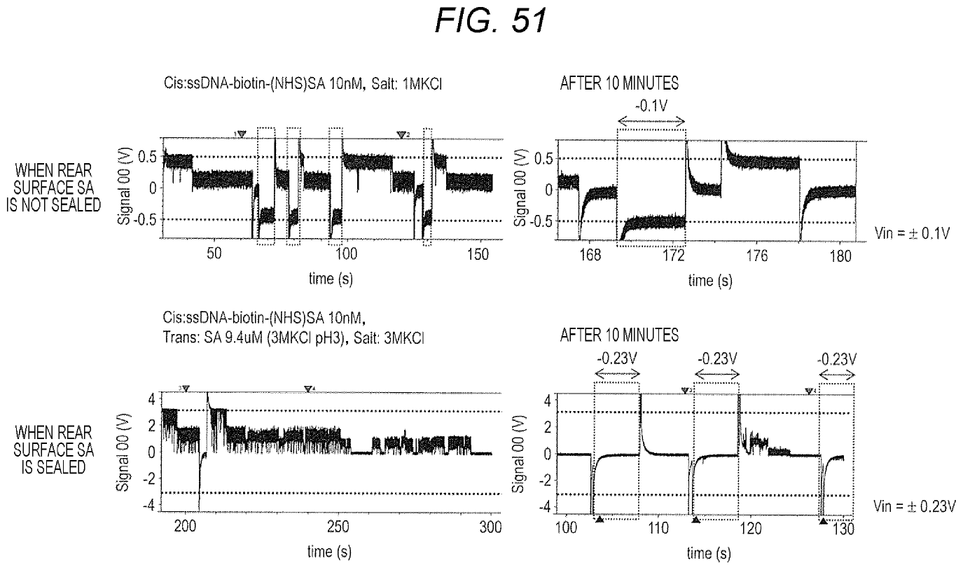

Meanwhile, as illustrated in FIG. 50, the adapter molecule 900 described above may comprise a three-dimensional structure formation domain 114 and a three-dimensional structure formation inhibiting oligomer 115 hybridizing with the three-dimensional structure formation domain 114 in the single-stranded nucleic acid region 901B. The three-dimensional structure formation domain 114 is located on the side of the terminus from the molecular motor detachment induction portion 702 in the single-stranded nucleic acid region 901B. In a case where the adapter molecule 900 including the three-dimensional structure formation domain 114 and the three-dimensional structure formation inhibiting oligomer 115 is used, the three-dimensional structure formation domain 114 forms a three-dimensional structure in the second liquid tank 104B in the state illustrated in each of FIGS. 47 to 49. When a three-dimensional structure is formed in the vicinity of the end of the biomolecule-adapter molecule complex 905 in the second liquid tank 104B, it is possible to reliably prevent the biomolecule-adapter molecule complex 905 from falling off from the nanopore 101.

### EXAMPLES

Hereinafter, the present invention will be described in more detail by examples, and the technical scope of the present invention is not limited to the following examples.

### Reference Examples

As disclosed in JP 2010-230614 A, in some cases, it may be possible to take means for binding a molecule having a diameter larger than that of a nanopore, such as streptavidin (SA), to both termini of a DNA strand to be analyzed and controlling a voltage. However, in the case of this method, SA on the side of the second liquid tank 104B (also referred to as a trans chamber) needs to be bound after the DNA strand passes through the nanopore. In order to bind one molecule of SA dissolved on the side of the second liquid tank 104B to one molecule of the DNA strand that has passed through the nanopore, it is necessary to wait for a sufficient time until binding or to dissolve SA to a sufficient concentration.

In the present reference example, the results of the experiment of binding SA to the DNA strand in the second liquid tank 104B are shown. The experiment in the second liquid tank 104B was performed at salt concentrations of 1 M KCl and 3 M KCl. As a biomolecule, single-stranded DNA 80mer-modified with biotin at both termini was used. The single-stranded DNA and SA were reacted in advance at a concentration at which only SA was bound to one biotin, and the reactant was passed through the nanopore.

The current value measured in the absence of the biomolecule 109 was used as a reference (pore current), and trapping, passing, and separation of DNA into the nanopore were determined from the presence or absence of a decrease in current value. The upper part of FIG. 51 illustrates results when only the measurement solution is put in the second liquid tank 104B. SA-bound ssDNA was introduced into the chamber, and immediately after the start of measurement, a decrease in ion current derived from DNA (blocking current) was confirmed. The blocking current continued to block the nanopore without being eliminated. This indicates that SA bound to the terminus of DNA cannot pass due to a diameter equal to or larger than the diameter of the nanopore and is trapped in the nanopore. Here, when the voltage is inverted, a current value derived from the pore diameter is recovered. Since the terminus of the DNA present in the second liquid tank 104B remains as a single-strand, it is considered that the DNA has come out to the first liquid tank 104A by electrophoresis.

Here, SA was dissolved in the second liquid tank 104B so as to have a final concentration of 9 μM. DNA was similarly reacted at a concentration at which one SA was bound, and was used for nanopore measurement. When DNA is introduced into the first liquid tank 104A, a pore blocking phenomenon is similarly confirmed. Here, when the voltage is inverted immediately after the blocking, a phenomenon in which SA is recovered to the pore current similarly to the case where SA is not introduced into the second liquid tank 104B is observed. On the other hand, when the voltage was inverted after waiting for 10 minutes after the confirmation of the blocking, it was confirmed that the measured ion current was not returned to the pore current and was continuously blocked (the lower part of FIG. 51).

When the time from the confirmation of the blocking derived from SA-attached DNA to the voltage inversion was changed, a standby time of at least 10 minutes was required, and even when the standby time was 10 minutes or longer, the binding was not necessarily possible. That is, it was found that the time for binding SA to the terminus of the DNA strand in the second liquid tank 104B was long, which was a factor that led to inhibition of the efficiency of measurement. In addition, it was determined that the criterion for determining whether or not the binding of SA to the terminus of the DNA strand has been completed is also ambiguous.

### Example 1

In the present example, the adapter molecule 300 illustrated in FIG. 13 was actually designed, and the effectiveness of the three-dimensional structure by the three-dimensional structure formation domain 114 was evaluated.

Specifically, DNAs having the sequences shown in Table 1 were designed as the biomolecule 109 and the primer 131. iSpC3 was arranged as the spacer 304 at the position indicated by “Z”. In addition, streptavidin was used as the fall-off prevention portion 113. In addition, as the sequence of the three-dimensional structure formation domain 114, the telomeric sequence shown in Table 1 was used. The double-stranded region 201 and subsequent single-stranded nucleic acid regions 301A and 301B were designed as shown in the table.

TABLE 1

| Name | Sequence (5′-3′) |
|---|---|
| Primer | GCGGTGTTCTGTTGGTGCTGATATT (SEQ ID NO: 1) |
| Biomolecule | ACGAATTCGAGCTCGGTACCCGGGGATCCTCTAGAGTCGACCTGCAGGC |
| | ATGCAA GCTTGGCACTGGCCGTCGTTTTACA (SEQ ID NO: 2) |
| Sequence having single- | AGCCAGCGTCCGGGGATGAGCTACTCCCGGTTTTTTTTTTTTTTTTTTT |
| stranded nucleic acid | TTZZZZGCGGTGTTCTGTTGGTGCTGATATT-biotin (SEQ ID NO: |
| region 301A | 3) |
| Sequence having single- | TTTTTTTTTTTTTTTTTTTTTTTTTTTTTTTTTTTTTTTTTTTTTTTTT |
| stranded nucleic acid | TTTCGG |
| region 301B | CTCCACTCTGAATGGGTTAGGGTCAGGGCACGGGCTAGGGACTGGGATA |
| | GGGTTA TTCTGTCCGGGAGTAGCTCATCCCCGGACGCTGGCTT (SEQ |
| | ID NO: 4) |
| Three-dimensional structure | ACAGAATAACCCTATCCCAGTCCCTAGCCCGTGCCCTGACCCTAACCC |
| formation inhibiting | (SEQ ID NO: 5) |
| oligomer | |

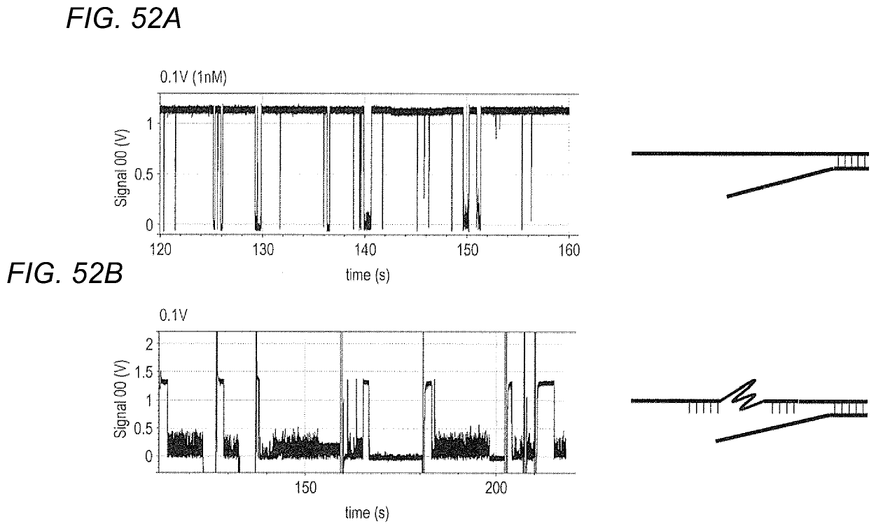

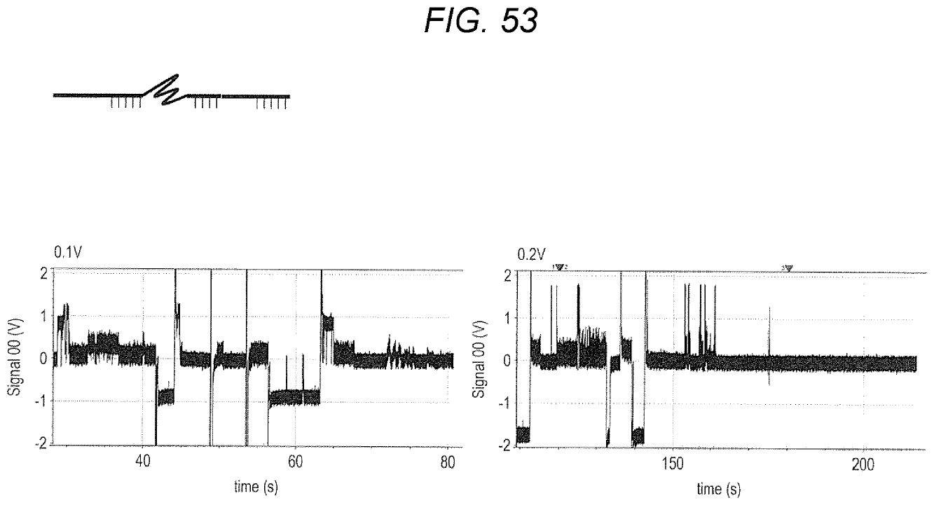

FIG. 52 shows data of an experiment to confirm whether or not the biomolecule 109 can reciprocate between the fall-off prevention portion 113 and the three-dimensional structure by forming the three-dimensional structure by the three-dimensional structure formation domain 114 in the biomolecule 109 to which the adapter molecule 300 designed as described above was bound. In this experiment, a buffer solution in the first liquid tank 104A and the second liquid tank 104B separated by the thin film 102 having the nanopore 101 was set as a solution having a salt concentration usually used for the nanopore measurement. Here, the polymerase and the primer were not bound.

Here, since it was confirmed by the experiment shown in [Reference Example] that the nanopore is blocked by the binding of SA, it was confirmed that the nanopore 101 is blocked by the telomere structure. FIG. 52 shows changes in ion currents when an adapter having no telomere structure of the three-dimensional structure formation domain 114 and an adapter having a telomere structure are measured. A signal acquired when there is no telomere structure is shown in FIG. 52(a), and a signal acquired when there is a telomere structure is shown in FIG. 52(b). When there is no telomere structure, it was observed that the passing signal, that is, the blocking signal is spontaneously recovered to the base current. On the other hand, when a sample having a telomere structure was used, the current was not spontaneously returned to the base current after confirmation of the blocking signal. Thereafter, it was confirmed that the voltage was inverted to return to the base current.

FIG. 53 shows results obtained by melting a single strand having a telomere structure in a measurement solution and measuring a nanopore. As a result, a signal to keep blocking at 0.1 V was confirmed. It can be said that the blocking of the nanopore confirmed in FIG. 52(b) is derived from the telomere structure formed in the adapter molecule. On the other hand, it was also confirmed that when the measurement voltage was increased, a passing signal was generated. This indicates that the withstand voltage of the telomere structure is around 0.2 V.

Based on the above results, an applied voltage of 0.1 V was used for an experiment to confirm that the biomolecule can be trapped in the nanopore with SA and the telomere structure as fall-off prevention portions 113.

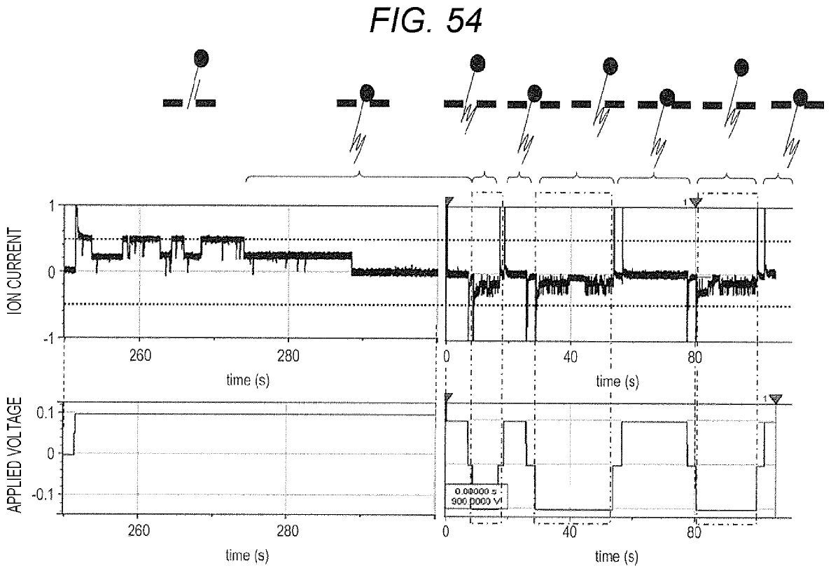

FIG. 54 shows results of confirming whether a biomolecule can be trapped in a nanopore using a sample in which an adapter molecule having a telomere structure as the three-dimensional structure formation domain 114 is ligated to a biomolecule. After the ligation, SA was mixed and incubated at 37 degrees so that SA could be bound to the terminus of the single-stranded nucleic acid region 301A. After about 25 seconds from the introduction of the sample, a phenomenon in which the ion current was decreased and was not recovered to the base current was confirmed. After waiting for 30 seconds, the applied voltage was inverted, but the current value was not returned to the base current. Furthermore, after about 5 seconds, the applied voltage was returned, but the current value before voltage inversion was acquired without being returned to the base current. These operations were repeated three times, but the applied voltage was not returned to the base current.

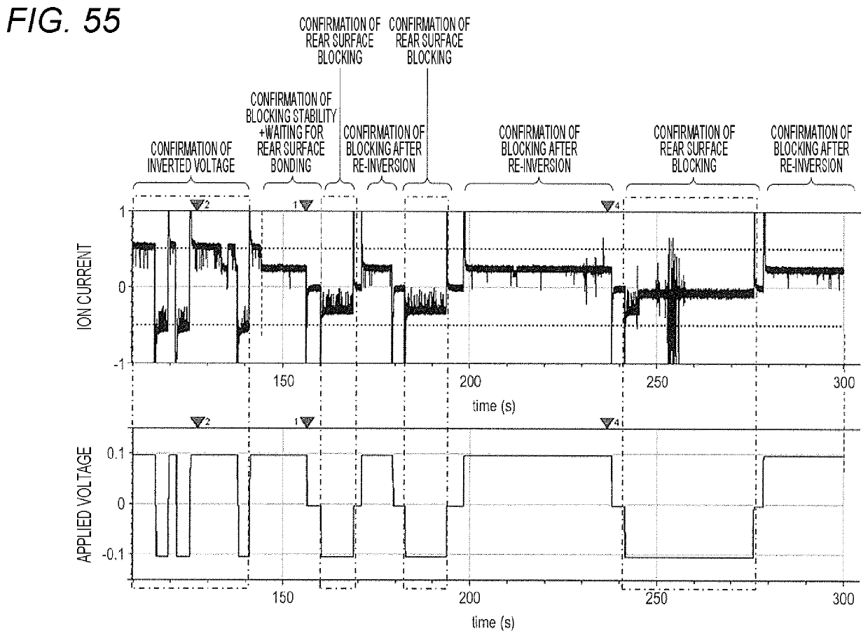

FIG. 55 also shows experimental examples performed with similar different pores and different samples. Similarly, even when the voltage inversion is performed before the introduced sample is blocked, only the base current is confirmed (<30 seconds) , but once the blocking is confirmed, the blocking current is maintained without being returned to the base current.

From the above, the following is presumed. As illustrated in the schematic view in the upper part of FIG. 54, it is considered that the single-stranded DNA (the biomolecule 109) is remained in the nanopore between SA bound to the single-stranded nucleic acid region 301A and the three-dimensional structure formed by the passing of the single-stranded nucleic acid region 301B through the nanopore 101. From the above, it was concluded that a configuration capable of implementing a rapid reciprocating motion between the fall-off prevention portion 113 bound to the first control strand and the three-dimensional structure formed by the three-dimensional structure formation domain 114 was realized.

### Example 2

In the present example, an adapter molecule including a molecular motor detachment induction portion having a smaller binding force to a biomolecule than a binding force to the molecular motor was designed, and whether the molecular motor can be separated by the molecular motor detachment induction portion was examined.

Specifically, as shown in Table 2, “Primer Oligo 23 nt” was designed as the primer 131, and adapter molecules comprising three types of molecular motor detachment induction portions were designed.

TABLE 2

| Name | Sequence | x type |
|---|---|---|
| Primer | TATCAGCACCAACAGAACACCGC (SEQ ID NO: 6) | |
| Oligo 23 | | |
| nt | | |
| iSp18x4_ | TTTTTTTTTTTTTTTTTTTTXXXXTTCCTCACTTCTCCATTTATCCTA | isp18 |
| T20_Deb18 | CAGAGGATCAGGTGGCCATCTTGZZZZGCGGTGTTCTGTTGGTGCTGATA | |
| iSp9x4_ | TTTTTTTTTTTTTTTTTTTTXXXXTTCCTCACTTCTCCATTTATCCTACAGAG | Isp9 |
| T20_Deb18 | GATCAGGTGGCCATCTTGZZZZGCGGTGTTCTGTTGGTGCTGATA | |
| i5NitIndx4_ | TTTTTTTTTTTTTTTTTTTTXXXXTTCCTCACTTCTCCATTTATC | 5Nitroin |
| T20_Deb18 | CTACAGAGGATCAGGTGGCCATCTTGZZZZGCGGTGTTCTGTTGGTGCTGATA | dole |

In Table 2, X indicates a molecular motor detachment induction portion. The position indicated by Z is a spacer composed of iSpC3.

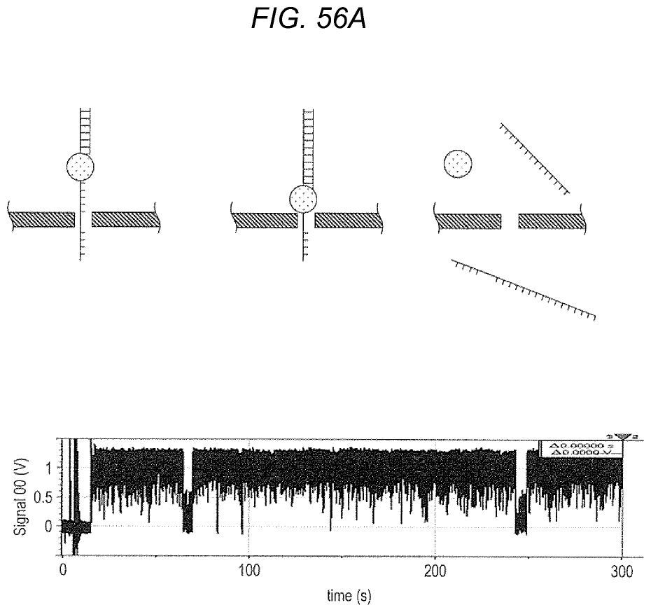

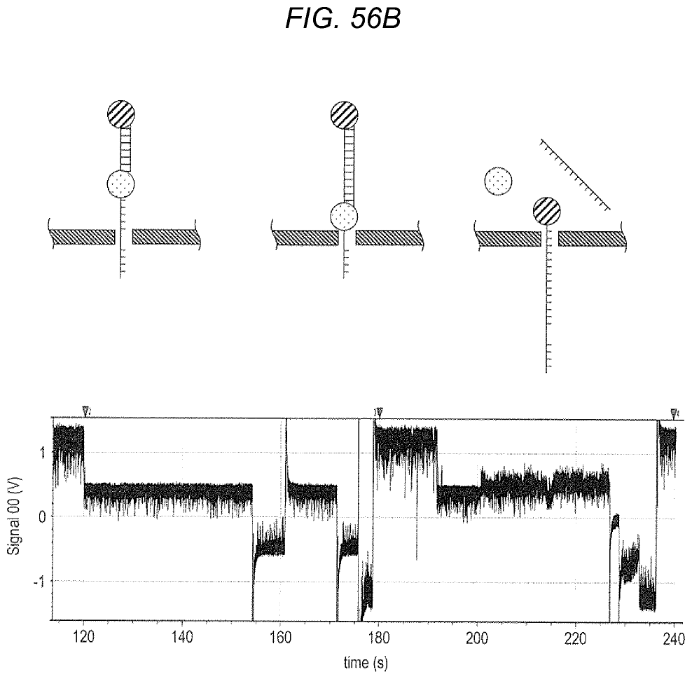

The results of observing the nanopore passing signal in the presence of the molecular motor using the primer 131 and the adapter molecule described in Table 2 are shown in FIGS. 56a and 56b. In addition, in the present example, iSp18x4_T20_Deb18 was typically used as a template. In the adapter molecule, the molecular motor detachment induction portion is present at the portion indicated by X. Similarly to the observation of the nanopore passing signal using the template having no molecular motor detachment induction portion, an early passing signal having a blocking time of 1 ms or shorter, which is considered to be an unzip signal of the primer and a passing signal having a blocking time of 1 to 100 ms, which is considered to be a signal derived from conveyance by polymerase, were confirmed. Here, a signal confirmed in the absence of dNTPs, that is, a signal at which polymerase was trapped in the nanopore while being bound to the template, in other words, a signal at which the nanopore was blocked by polymerase and was maintained in this state, was not confirmed. When the same measurement was performed using a molecule in which SA was bound to the single strand used in FIG. 56a, a signal at which blocking was maintained was confirmed (FIG. 56b).

From these results, the following is presumed. As illustrated in the schematic view, from the results of FIG. 56a, it was considered that when the molecular motor that started the extension reaction from the primer reached the molecular motor detachment induction strand, the molecular motor was detached from the single strand, the synthesized strand was started to be peeled off, and the template was passed through the nanopore. From the results of FIG. 56b, it was considered that when the template assumed from the results of FIG. 56a was passed through the nanopore, the passing was not realized due to being trapped by SA because SA was bound to the terminus of the template. Since it is confirmed that the voltage is returned to the base current when the voltage is inverted after the confirmation of the blocking, it is considered that a state in which the single strand is trapped by SA is realized.

From the above, it was shown that it is possible to positively stop the conveyance of the molecular motor from the template and to release the binding to the single-stranded DNA as the template by disposing the molecular motor detachment induction portion.

### Example 3

In the present example, in a case where a plurality of sets of a primer binding site and a molecular motor binding portion were provided as in the adapter molecule 400 illustrated in FIG. 17, a preferred interval between the adjacent primer binding sites was examined.

Specifically, in a configuration having a primer binding site and a spacer (a region composed of an abasic site) downstream thereof, an adapter molecule was designed by setting the interval between the adjacent primer binding sites to a base length of 15, 25, 35, or 75. A buffer solution containing the designed adapter molecule and molecular motor (polymerase) was prepared, and electrophoresis was performed after the molecular motor was bound to the adapter molecule.

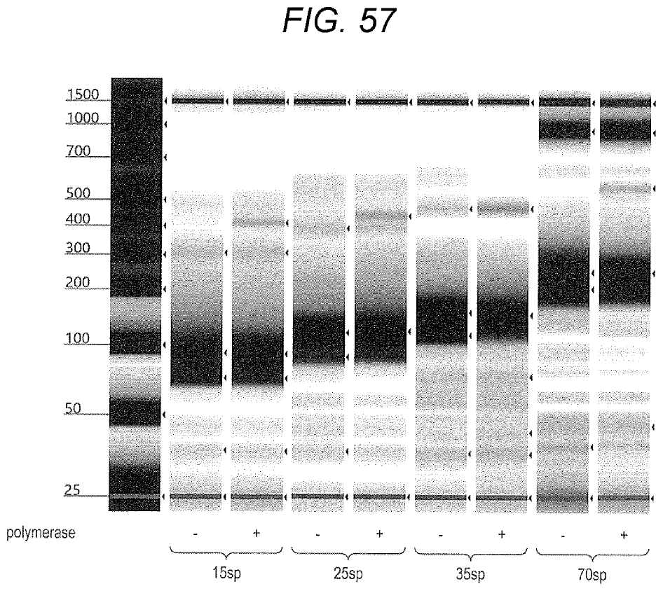

The results are shown in FIG. 57. In FIG. 57, “polymerase: +” and “polymerase: −” indicate whether the experiment was performed in the state where polymerase as a molecular motor was present in the buffer solution (+) or not (−). When polymerase is bound to the adapter molecule under any condition, a new band appears at a position different from the band position appearing only at the time of annealing. As shown in FIG. 57, new bands were observed in the presence of polymerase in all the adapter molecules designed in the present example. It was clarified from this that polymerase as a molecular motor could be bound even when the interval between the adjacent primer binding sites was a base length of 15, 25, 35, or 75.

### Example 4

In the present example, it was confirmed that as in the adapter molecule 900 illustrated in FIG. 46, an adapter molecule comprising a molecular motor detachment induction portion having a smaller binding force to a biomolecule than a binding force to the molecular motor, and a plurality of combinations of a primer binding portion, a molecular motor binding portion, and a spacer formed between the primer binding portion and the molecular motor binding portion was designed, and whether repeated conveyance control of the target molecule was possible.

Specifically, as shown in Table 3, “Primer Oligo 23 nt” was designed as the primer 131, and “Tandem primer template” was designed as the adapter molecule so that “Primer Oligo 23 nt” was bound at 3 positions. The length of the molecule to be analyzed was 69mer. In addition, the interval between the adjacent primer binding sites was 15mer.

TABLE 3

| Name | Sequence |
|---|---|
| Primer | TATCAGCACCAACAGAACACCGC (SEQ ID NO: 6) |
| Oligo 23 | |
| nt | |
| Tandem | TTTTTTTTTTTTXXXXAATCGAGACTGTAGACAAAGGCTTC |
| primer | CTCACTTCTCCATTTATCCTACAGAGGATCAGGTGGCCATC |
| template | TT |
| | GZZZZGCGGTGTTCTGTTGGTGCTGATATTTTTTTTTTTTT |
| | TTZZZZGCGGTGTTCTGTTGGTGCTGATATTTTTTTTTTTT |
| | TT TZZZZGCGGTGTTCTGTTGGTGCTGATA |

In Table 3, X indicates a molecular motor detachment induction portion. The position indicated by Z is a spacer consisting of iSpC3.

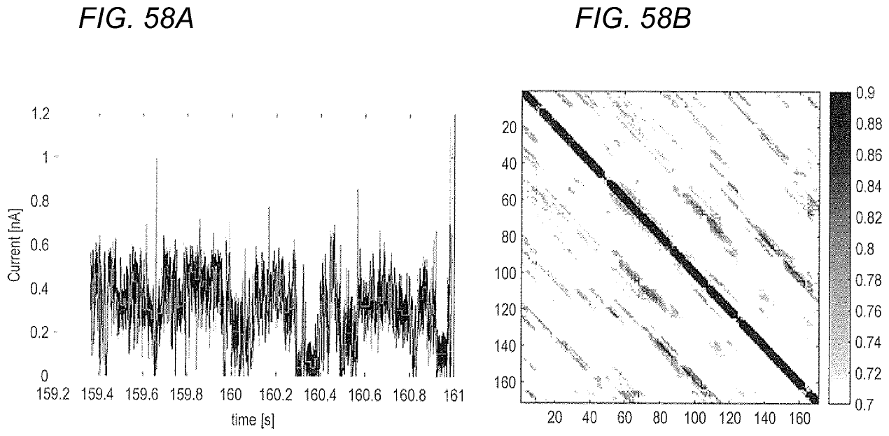

The results of observing the nanopore passing signal in the presence of the molecular motor using the primer 131 and the adapter molecule described in Table 3 are shown in FIG. 58. FIG. 58(a) is a representative view of the measured blocking signal. A portion where the current value is particularly high indicates that the resistance of the nanopore is the lowest, and is considered to indicate a portion of iSpC3 in the “Tandem primer template”.

Here, Dotplot analysis was performed to examine that waveforms obtained by reading the same region were reflected in the acquired waveforms. In the Dotplot, the division of the waveform formed by the current value applied to each level, for example, every 10 levels is analyzed by a dynamic expansion/contraction method, and as a result, a higher score is output as the similarity increases (FIG. 58(b)). In FIG. 58(b), since the diagonal line indicates the similarity at the same point, the score of perfect coincidence is output. On the other hand, for example, the level of 80 to 100 and the level of 120 to 140 indicate a preferred match between different locations.

Level extraction was performed on the acquired waveforms, and locations with high similarities to each other were searched for with waveform divisions of the level of 30 by using the method described above. As for the level extraction, an average of the current values in an arbitrary time window was defined as a representative current level. The acquired Dotplot is as illustrated in FIG. 58(b). FIG. 58(b) roughly illustrates that the level of 0 to 60, the level of 60 to 120, and the level of 120 to 200 are similar to the total number of levels of 200. In addition, it is also illustrated that the second levels of 80 to 110 and 110 to 140 are similar.

When the sequence designed this time is repeatedly analyzed as intended, the region to be read is repeated three times. In addition, since the primer portion is read twice at the third repetition, the second line output as the similar waveform is shifted. Actually, as shown in FIG. 58(b), the second line to which this is reflected is output in this Dotplot analysis. From this result, it is shown that three repeated analyses were realized as designed.

From the above, it was shown that the plurality of primer binding portions were prepared and the molecular motor detachment induction portion was arranged, such that the conveyance by polymerase, the separation of polymerase, and unzip were automatically repeated as many times as the number of bounded primers without controlling the voltage, thereby performing highly accurate analysis of the target molecule.

### Reference Example 2

In the present Reference Example 2, a procedure of producing a nanopore to which the present invention is applied by a semiconductor micromachining technique will be described. First, Si₃N₄/SiO₂/Si₃N₄ is formed on a surface of an 8 inch Si wafer having a thickness of 725 μm in the order of thicknesses of 12 nm/250 nm/100 nm. In addition, Si₃N₄ having a thickness of 112 nm is formed on a rear surface of the Si wafer.

Next, Si₃N₄ at the uppermost portion of the Si wafer is removed by reactive ion etching in a 500 nm square. Similarly, Si₃N₄ on the rear surface of the Si wafer is removed by reactive ion etching in a 1,038 μm square. The rear surface of the Si substrate exposed by etching is further etched with tetramethylammonium hydroxide (TMAH). During the Si etching, it is preferable to cover the wafer surface with a protective film (ProTEKTMB3 primer and ProTEKTMB3, Brewer Science, Inc.) in order to prevent etching of SiO on the surface side. SiO of the intermediate layer may be polysilicon.

Next, after the protective film is removed, the SiO layer exposed in a 500 nm square is removed with a BHF solution (HF/NH₄F=1/60, 8 ). Therefore, a partition body in which the thin film Si3N4 having a film thickness of 12 nm is exposed is obtained. In a case where polysilicon is selected for a sacrificial layer, the thin film is exposed by etching with KOH. At this stage, the thin film is not provided with a nanopore.

The formation of the nanopore can be performed, for example, by the following procedure. Before the partition body is set in a device for biomolecule analysis or the like, the Si₃N₄ thin film is hydrophilized by Ar/O2 plasma (SAMCO Inc., Japan) under conditions of 10 W, 20 sccm, 20 Pa, and 45 sec. Next, the partition body is set in the device for biomolecule analysis. Thereafter, the upper and lower liquid tanks with the thin film interposed therebetween are filled with a solution containing 1 M KCl and 1 mM Tris-10 mM EDTA and having a pH of 7.5, and an electrode is introduced into each of the liquid tanks.

The voltage is applied not only at the time of forming the nanopore but also at the time of measuring the ion current flowing through the nanopore after the nanopore is formed. Here, the liquid tank located on the lower side is referred to as a cis tank, and the liquid tank located on the upper side is referred to as a trans tank. In addition, the voltage Vcis applied to the electrode on the cis tank side is set to 0 V, and the voltage Vtrans is applied to the electrode on the trans tank side. The voltage Vtrans is generated by a pulse generator (for example, 41501 B SMUAND Pulse Generator Expander, Agilent Technologies, Inc.).

The current value after the pulse application can be read by an ammeter (for example, 4156 B PRECISION SEMICONDUCTOR ANALYZER, Agilent Technologies, Inc.). A current value condition (threshold current) is selected according to the diameter of the nanopore formed before the application of the pulse voltage, and the target diameter can be obtained while sequentially increasing the diameter of the nanopore.

The diameter of the nanopore was estimated from the ion current value. Condition selection criteria are as shown in Table 4.

TABLE 4
Voltage application conditions

| Nanopore diameter before | Non-opening | | |
|---|---|---|---|
| pulse voltage application | to 0.7 nmΦ | to 1.4 nmΦ | to 1.5 nmΦ |
| Applied voltage (V_{cis}) [V] | 10 | 5 | 3 |
| Initial application time [s] | 0.001 | 0.01 | 0.001 |
| Threshold current | 0.1 nA/ | 0.6 nA/ | 0.75 nA/ |
| | 0.4 V | 0.1 V | 0.1 V |

Here, the n-th pulse voltage application time to (where, an integer of n>2) is determined by the following equation.

tₙ=10^{-3+(1/6)(n-1)}−10^{-3+(1/6)(n-2)} For n>2 [Math. 1]

As described above, it was shown that the nanopore having a desired opening diameter was appropriately produced by a specific method. The formation of the nanopore can also be performed by irradiation with electron beam by TEM in addition to a method of applying a pulse voltage (A. J. Storm et al., Nat. Mat. 2 (2003)).

1-39. (canceled)

40. A biomolecule analysis method comprising steps of:
applying a voltage between a first liquid tank and a second liquid tank facing each other with a thin film having a nanopore interposed therebetween in a state where the first liquid tank is filled with an electrolyte solution comprising a biomolecule-adapter molecule complex, a molecular motor boundable to a molecular motor binding portion of the adapter molecule, and a primer capable of hybridizing with a primer binding portion of the adapter molecule and the second liquid tank is filled with an electrolyte solution to generate a potential gradient at which the first liquid tank is set to a negative or ground potential and the second liquid tank is set to a positive potential wherein the biomolecule-adapter molecule complex comprises a biomolecule to be analyzed and an adapter molecule directly or indirectly bound to at least one terminus of the biomolecule, wherein the adapter molecule consists of a single-stranded nucleotide and comprises a plurality of sets of a molecular motor binding portion to which a molecular motor binds and a primer binding portion, with which a primer hybridizes, on a side of a 3′-terminus from the molecular motor binding portion; and
measuring a signal generated when the biomolecule-adapter molecule complex is convayed between the second liquid tank and the first liquid tank through the nanopore,
wherein in the step of measuring of the signal, the measurement is repeated, the measurement being performed by synthesizing a complementary strand from the primer hybridizing with the primer binding portion by the molecular motor located closest to the nanopore to convay the biomolecule-adapter molecule complex from the second liquid tank to the first liquid tank so as to measure a signal generated when the biomolecule-adapter molecule complex passes through the nanopore, and then, convaying the biomolecule-adapter molecule complex having a complementary strand from the first liquid tank to the second liquid tank to peel off the complementary strand and synthesizing the complementary strand by the molecular motor located closest to the nanopore again to convay the biomolecule-adapter molecule complex from the second liquid tank to the first liquid tank so as to measure a signal.

41. A biomolecule analysis method comprising steps of:
applying a voltage between a first liquid tank and a second liquid tank facing each other with a thin film having a nanopore interposed therebetween in a state where the first liquid tank is filled with an electrolyte solution comprising a biomolecule-adapter molecule complex, a molecular motor boundable to a molecular motor binding portion of the biomolecule-adapter molecule complex, and a primer capable of hybridizing with a primer binding portion of the biomolecule-adapter molecule complex and the second liquid tank is filled with an electrolyte solution to generate a potential gradient at which the first liquid tank is set to a negative or ground potential and the second liquid tank is set to a positive potential, wherein the biomolecule-adapter molecule complex comprises a biomolecule to be analyzed and an adapter molecule directly or indirectly bound to at least one terminus of the biomolecule, wherein the adapter molecule comprises a molecular motor detachment induction portion of which a binding force to a molecular motor is smaller than that of the biomolecule; and
measuring a signal generated when the biomolecule-adapter molecule complex is convayed between the second liquid tank and the first liquid tank through the nanopore, wherein in the step of measuring of the signal, the molecular motor synthesizes a complementary strand from the primer hybridizing with the primer binding portion to convay the biomolecule-adapter molecule complex from the second liquid tank to the first liquid tank so as to separate the molecular motor by the molecular motor detachment induction portion in the biomolecule-adapter molecule complex.

42. The biomolecule analysis method according to claim 40, wherein the adapter molecule further comprises a spacer to which the molecular motor is not bound being provided between the molecular motor binding portion and the primer binding portion.

43. The biomolecule analysis method according to claim 40, wherein the adapter molecule further comprises a fall-off prevention portion having a diameter larger than that of a nanopore in an analyzer for the biomolecule at an end opposite to an end that directly or indirectly binds to the biomolecule.

44. The biomolecule analysis method according to claim 43, wherein the fall-off prevention portion is a molecule bondable to the single-stranded nucleic acid region or a hairpin structure formed in a complementary region in the single-stranded nucleic acid region.

45. The biomolecule analysis method according to claim 40, wherein the adapter molecule further comprises:
a double-stranded nucleic acid region consisting of base sequences complementary to each other and comprising one end that directly or indirectly binds to the biomolecule to be analyzed; and
a single-stranded nucleic acid region linked to an other end different from the one end of the double-stranded nucleic acid region, having the 3′-terminus, and comprising a plurality of sets of the molecular motor binding portion and the primer binding portion.

46. The biomolecule analysis method according to claim 40, wherein the adapter molecule further comprises:
a double-stranded nucleic acid region consisting of base sequences complementary to each other and comprising one end that directly or indirectly binds to the biomolecule to be analyzed; and
a pair of single-stranded nucleic acid regions linked to an other end different from the one end of the double-stranded nucleic acid region and consisting of base sequences not complementary to each other,
wherein the plurality of sets of the molecular motor binding portion and the primer binding portion are located in a single-stranded nucleic acid region having the 3′-terminus of the pair of single-stranded nucleic acid regions.

47. The biomolecule analysis method according to claim 46, wherein a single-stranded nucleic acid region having a 5′-terminus of the pair of single-stranded nucleic acid regions comprises a three-dimensional structure formation domain.

48. The biomolecule analysis method according to claim 47, wherein the adapter molecule further comprises a three-dimensional structure formation inhibiting oligomer comprising a base sequence complementary to at least a portion of the three-dimensional structure formation domain.

49. The biomolecule analysis method according to claim 48, wherein the three-dimensional structure formation inhibiting oligomer hybridizes with at least a portion of the three-dimensional structure formation domain, and a side of a terminus from a portion with which the three-dimensional structure formation inhibiting oligomer hybridizes is a single strand.

50. The biomolecule analysis method according to claim 46, wherein a single-stranded nucleic acid region having a 5′-terminus of the pair of single-stranded nucleic acid regions comprises a molecular motor detachment induction portion of which a binding force to a molecular motor is smaller than that of the biomolecule.

51. The biomolecule analysis method according to claim 41, wherein the molecular motor detachment induction portion is a carbon chain that does not contain a phosphodiester bond or an abasic sequence portion.

52. The biomolecule analysis method according to claim 41, wherein the adapter molecule further comprises a three-dimensional structure formation domain consisting of a single-stranded nucleotide on a side of a 5′-terminus from the molecular motor detachment induction portion.

53. The biomolecule analysis method according to claim 41, wherein the adapter molecule further comprises:
a double-stranded nucleic acid region consisting of base sequences complementary to each other and comprising one end that directly or indirectly binds to the biomolecule to be analyzed; and
a single-stranded nucleic acid region linked to an other end different from the one end of the double-stranded nucleic acid region, having a 5′-terminus, and including the molecular motor detachment induction portion.

54. The biomolecule analysis method according to claim 41, wherein the adapter molecule further comprises:
a double-stranded nucleic acid region consisting of base sequences complementary to each other and comprising one end that directly or indirectly binds to the biomolecule to be analyzed; and
a pair of single-stranded nucleic acid regions linked to an other end different from the one end of the double-stranded nucleic acid region and consisting of base sequences not complementary to each other,
wherein the molecular motor detachment induction portion is located in a single-stranded nucleic acid region having a 5′-terminus of the pair of single-stranded nucleic acid regions.

55. The biomolecule analysis method according to claim 52, wherein the adapter molecule further comprises a three-dimensional structure formation inhibiting oligomer comprising a base sequence complementary to at least a portion of the three-dimensional structure formation domain.

56. The biomolecule analysis method according to claim 55, wherein the three-dimensional structure formation inhibiting oligomer hybridizes with at least a portion of the three-dimensional structure formation domain, and a side of a terminus from a portion with which the three-dimensional structure formation inhibiting oligomer hybridizes is a single strand.

57. The biomolecule analysis method according to claim 54, wherein a single-stranded nucleic acid region having a 3′-terminus at an end of the pair of single-stranded nucleic acid regions includes a fall-off prevention portion having a diameter larger than that of a nanopore in an analyzer for the biomolecule.

58. The biomolecule analysis method according to claim 57, wherein the fall-off prevention portion is a molecule bondable to the single-stranded nucleic acid region or a hairpin structure formed in a complementary region in the single-stranded nucleic acid region.

59. The biomolecule analysis method according to claim 54, wherein a single-stranded nucleic acid region having a 3′-terminus at an end of the pair of single-stranded nucleic acid regions comprises a molecular motor binding portion to which a molecular motor binds.

60. The biomolecule analysis method according to claim 59, wherein the single-stranded nucleic acid region comprises the molecular motor binding portion comprises a primer binding portion with which a primer hybridizes on a side of the 3′-terminus from the molecular motor binding portion.

61. The biomolecule analysis method according to claim 60, wherein the adapter molecule further comprises a spacer to which the molecular motor is not bound being provided between the molecular motor binding portion and the primer binding portion.

62. The biomolecule analysis method according to claim 54, wherein a single-stranded nucleic acid region having a 3′-terminus at an end of the pair of single-stranded nucleic acid regions comprises a plurality of sets of a molecular motor binding portion to which a molecular motor binds and a primer binding portion with which a primer hybridizes on a side of the 3′-terminus from the molecular motor binding portion.

63. The biomolecule analysis method according to claim 62, wherein the adapter molecule further comprises a spacer to which the molecular motor is not bound being provided between the molecular motor binding portion and the primer binding portion.
